(19) United States
(12) Patent Application Publication
(10) Pub. No.: US 2026/0157970 A1
(43) Pub. Date: Jun. 11, 2026

(54) PHARMACEUTICAL COMPOSITIONS

(71) Applicant: Rigel Pharmaceuticals, Inc., South San Francisco, CA (US)

(72) Inventors: Lu Chou, Millbrae, CA (US); Nandan Oza, Sunnyvale, CA (US)

(73) Assignee: Rigel Pharmaceuticals, Inc., South San Francisco, CA (US)

(21) Appl. No.: 19/410,904

(22) Filed: Dec. 5, 2025

Related U.S. Application Data
(60) Provisional application No. 63/729,260, filed on Dec. 6, 2024.

(51) Int. Cl. A61K 9/20; A61K 31/675; A61K 45/06

(52) U.S. Cl. CPC A61K 9/2077; A61K 9/2009; A61K 9/2018; A61K 9/2054; A61K 9/2095; A61K 31/675; A61K 45/06

(57) ABSTRACT

Provided herein is a pharmaceutical composition comprising 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a pharmaceutically acceptable salt thereof as an active agent, one or more intragranular excipients and one or more extragranular excipients.

### RELATED APPLICATIONS

This application claims the benefit of U.S. Provisional Application No. 63/729,260, filed Dec. 6, 2024, the disclosure of which is incorporated herein by reference in its entirety.

### FIELD

Provided herein are pharmaceutical compositions comprising 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof as an active agent, one or more intragranular excipients and one or more extragranular excipients. In certain embodiments, the pharmaceutical compositions are used as a medicament for treating, preventing, and/or ameliorating auto-immune diseases, inflammatory disorders, cardiovascular diseases, nerve disorders, neurodegenerative disorders, allergic disorders, asthma, pancreatitis, multi-organ failure, kidney diseases, platelet aggregation, cancer, transplantation, sperm motility, erythrocyte deficiency, graft rejection, lung injuries, respiratory diseases, ischemic conditions, and bacterial and viral infections.

### BACKGROUND ART

It has been reported that 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof is effective as an IRAK inhibitor and/or useful for delivering an IRAK inhibitor having a therapeutic use in treatment of diseases where IRAKs are implicated. See U.S. Pat. No. 11,370,787 B2.

There is a need to develop pharmaceutical compositions comprising 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof that have good manufacturability, dissolution, stability and bioavailability.

### SUMMARY

Provided herein is a pharmaceutical composition comprising 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof (Compound 1) as an active agent, one or more intragranular excipients and one or more extragranular excipients. In one embodiment, provided herein is an oral tablet comprising the pharmaceutical composition.

In one embodiment, the pharmaceutical composition comprises 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a pharmaceutically acceptable salt thereof as an active agent, one or more intragranular excipients and one or more extragranular excipients.

In one embodiment, the pharmaceutical composition comprises a 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt as an active agent, one or more intragranular excipients and one or more extragranular excipients.

In certain embodiments, the active agent used in the pharmaceutical composition is a solid form of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt. In one embodiment, the pharmaceutical composition comprises a solid form of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt as an active agent, one or more intragranular excipients and one or more extragranular excipients. In one embodiment, the pharmaceutical composition comprises Form E of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt as an active agent, one or more intragranular excipients and one or more extragranular excipients. In one embodiment, the pharmaceutical composition comprises a combination of Form E and Form D of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt as an active agent, one or more intragranular excipients and one or more extragranular excipients.

In one embodiment, the intragranular excipients comprise a diluent, a disintegrant, a glidant and a lubricant. In one embodiment, the extragranular excipients comprise a compression aid, a disintegrant, a glidant and a lubricant.

In certain embodiments, provided herein is a tablet comprising about 150 mg, about 250 mg, about 500 mg, about 750 mg or about 1000 mg of Compound 1. In certain embodiments, the tablets provided herein are tablets of about 150 mg, about 250 mg, about 500 mg, about 750 mg or 1000 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In certain embodiments, the tablets provided herein are tablets of about 150 mg, about 250 mg, about 500 mg, about 750 mg or 1000 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

In certain embodiments, provided herein is a tablet comprising about 150 mg or about 250 mg of Compound 1. In certain embodiments, the tablets provided herein are tablets of about 150 mg or about 250 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In certain embodiments, the tablets provided herein are tablets of about 150 mg or about 250 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

In certain embodiment, provided herein is a method for preparing a tablet comprising Compound 1. In certain embodiments, the method comprises blending Compound 1 with an intragranular excipient and an extragranular excipient and compressing with a compression tooling.

In one embodiment, the method comprises mixing Compound 1, and intragranular excipients including a diluent, a disintegrant, a glidant and a lubricant to obtain a blend; roller compacting the blend to form a ribbon; milling the ribbon to form granules; mixing the granules with extragranular excipients comprising a compression aid, a disintegrant, a glidant and a lubricant to obtain a final blend; and compressing the final blend with a compression tooling to obtain a tablet.

In certain embodiments, provided herein is a pharmaceutical composition comprising about 15 to about 35% Compound 1 as an active ingredient. In certain embodiments, provided herein is a pharmaceutical composition comprising about 60% to about 85% by weight of Compound 1 as an active ingredient.

In certain embodiments, provided herein are methods of treating, preventing, and/or ameliorating auto-immune diseases, inflammatory disorders, cardiovascular diseases, nerve disorders, neurodegenerative disorders, allergic disorders, asthma, pancreatitis, multi-organ failure, kidney diseases, platelet aggregation, cancer, transplantation, sperm motility, erythrocyte deficiency, graft rejection, lung injuries, respiratory diseases, ischemic conditions, and bacterial and viral infections comprising administering a pharmaceutical composition provided herein. In one embodiment, provided herein is a tablet comprising the pharmaceutical compositions.

### BRIEF DESCRIPTION OF DRAWINGS

FIG. 1 depicts an X-ray powder diffractogram for Form E of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 2 depicts a thermogravimetrical analysis (TGA) and a differential scanning calorimetry (DSC) thermogram plots of Form E of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 3 provides a PLM image of re-prepared Form E of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 4 provides compression profiles of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 5 provides plots for tabletability, compressibility, compactability, and disintegration profiles for prototype tablet formulations for 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 6 provides process steps in the manufacture of a demonstration batch compared to the reformulated blend.

FIG. 7 provides a particle size distribution for 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt demonstration batch granules.

FIG. 8 provides plots for tabletability, compression, friability, and disintegration profiles for 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt demonstration batch tablets.

FIG. 9 provides 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt demonstration batch weight distribution from composite sample of 100 tablets.

FIG. 10 provides a plot of compaction simulator force and relative density.

FIG. 11 provides a chart of granulation particle size distribution of 1000 g batch size tablet demonstration batch compared with a previous demonstration batch that was manufactured using a different model roller compactor.

FIG. 12 provides plots for tabletability, compression, friability, and disintegration profiles for 250 mg demonstration batch.

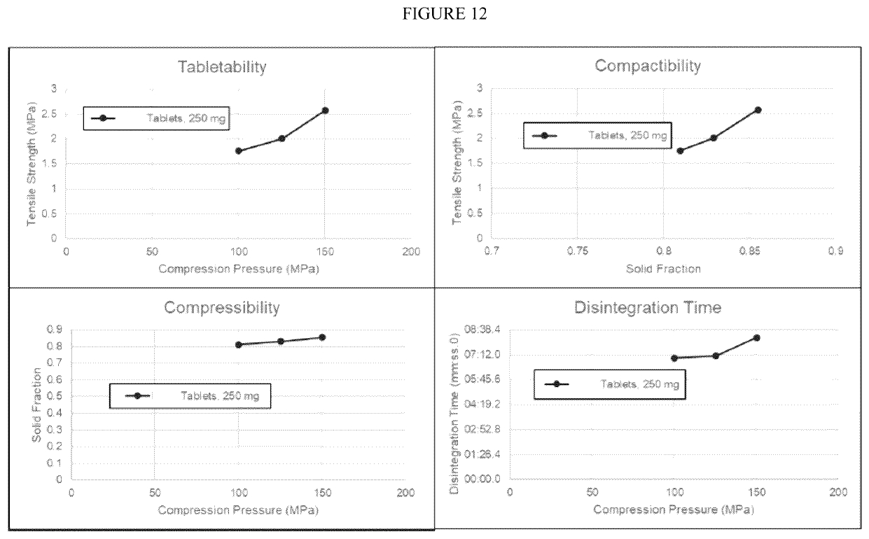

FIG. 13 provides a plot of friability of 250 mg demonstration batch tablets with compression pressure.

FIG. 14 provides a weight distribution chart for a composite sample of 250 mg demonstration batch tablet.

FIG. 15 provides plots for tabletability, compression, friability, and disintegration profiles for 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt prototype tablet formulations for Example 3.

FIG. 16 provides a plot showing increase in % weight with relative humidity for the tablet blend of Example 3.

FIG. 17 provides plots for tabletability, compression, friability, and disintegration profiles for 250, 500, and 750 mg scale-up tablets.

FIG. 18 depicts an X-ray powder diffractogram for Form D of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 19 provides a PLM image of re-prepared Form D of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 20 depicts a thermogravimetrical analysis (TGA) and a differential scanning calorimetry (DSC) thermogram plots of Form D of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 21 depicts a ¹H NMR spectrum of Form D of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

FIG. 22 depicts a FTIR spectrum of Form D of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

### DETAILED DESCRIPTION

The details of construction and the arrangement of components set forth in the following description or illustrated in the drawings are intended to describe non-limiting embodiments. Other embodiments and different ways to practice the invention are expressly included. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### Definitions

As used above, and throughout the description of the invention, the following terms, unless otherwise indicated, shall be understood to have the following meanings.

As used in this application, the singular form “a”, “an” and “the” include plural references unless the context clearly dictates otherwise. For example, the term “an intragranular excipient” includes one or more intragranular excipients.

“Compound 1” is meant to describe 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof. In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt (Compound 1A), including solid forms thereof.

The term “solid form” refers a crystal form or an amorphous form or a mixture thereof of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt. Exemplary solid forms of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt are described in a U.S. provisional application No. 63/729,262, titled “Solid Forms Of 1-(4-(4-((3-(3,6-Difluoropyridin-2-Yl)-1-((1r,4r)-4-Ethoxycyclohexyl)-1 h-Pyrazol-4-Yl)Carbamoyl)Thiazol-2-Yl)-1h-Pyrazol-1-Yl)Ethyl Phosphate Monosodium, And Their Pharmaceutical Compositions And Uses”, filed on Dec. 6, 2024, which is incorporated herein by reference in its entirety.

As used herein, d₁₀ refers to the particle size within a distribution of particles where 10 vol. % of the particles have a smaller particle size; d₅₀ refers to the particle size within a distribution of particles where 50 vol. % of the particles have a particle size that is larger and where 50 vol. % of the particles have a particle size that is smaller; and d₉₀ refers to the particle size within a distribution of particles where 90 vol. % of the particles have a smaller particle size.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of a compound provided herein, with or without other additional active agent, after the onset of symptoms of the particular disease.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease.

As used herein, and unless otherwise specified, a “therapeutically effective amount” of a compound is an amount sufficient to provide a therapeutic benefit in the treatment and/or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

The term “subject” or “patient” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example bulking agents, buffers, binders, fillers, disintegrants, lubricants, coatings, sweeteners, flavors, fatty acid esters, hydroxymethycellulose, polyvinyl pyrrolidine, colors, solvents, coatings, antioxidants, preservatives, wetting agents (surfactants) and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms “bulking agent”, and “buffer” are used in accordance with the plain and ordinary meaning within the art.

The term “intragranular excipients” refers to ingredients that are incorporated in the formulation prior to granulation, i.e., ingredients that are located internally in the granule structure.

The term “extragranular excipients” refers to ingredients that are incorporated after granulation, i.e. ingredients that are located externally to the granule structure.

As used herein, “administer” or “administration” refers to the act of physically delivering a substance as it exists outside the body into a subject. Administration includes all forms known in the art for delivering therapeutic agents, including but not limited to oral, topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

The term “co administering” as used herein with respect to an additional cancer therapeutic agents means that the additional cancer therapeutic agent may be administered prior to, consecutively with, or following the administration of a composition provided herein. In such combination therapy treatment, the second therapeutic agent(s) is administered by conventional methods.

It should be understood that the numerical values of the peaks of an X-ray powder diffraction pattern may vary slightly from one machine to another or from one sample to another, and so the values quoted are not to be construed as absolute, but with an allowable variability, such as ±0.2 degrees two theta (θ20) (see United State Pharmacopoeia, page 2228 (2003)).

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

### 1. Compound

In one embodiment, Compound 1 for use in the pharmaceutical compositions herein is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, which has the following structure:

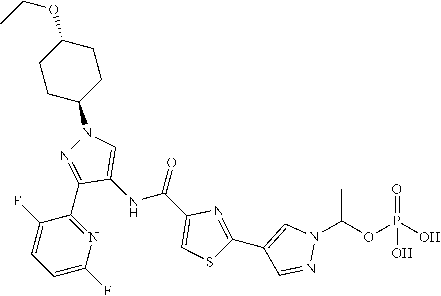 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

or a stereoisomer or mixture of stereoisomers, a pharmaceutically acceptable salt, tautomer, solvate, hydrate, co-crystal, clathrate, or polymorph thereof (Compound 1).

In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt (Compound 1A).

Exemplary methods for the synthesis of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt are described in U.S. Pat. No. 11,370,787 B2, and elsewhere herein.

In one embodiment, Compound 1 is a solid form of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt. Exemplary solid forms of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt include solid Forms D, E, J, L, U, O, Q, R, T, W, H, N, X, F, G, I, K, M, P, S, V and Y.

In one embodiment, the tablets provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt (API). The API exists in multiple solid forms generated post synthesis; and Forms D and E exhibit higher bulk density and reduced impurity levels relative to other forms. In one embodiment, Form E is used in the formulations provided herein due to its slightly higher bulk density and reduced “fluffiness” and better processing capability than the other solid forms of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt. In one embodiment, the tablets provided herein comprise solid Form E of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt. In one embodiment, the tablets provided herein comprise solid Forms E and D of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

In one embodiment, the API used in the tablets provided herein has a low bulk density. In one embodiment, the API used in the tablets provided herein is a highly aeratable and cohesive powder.

In one embodiment of the tablets provided herein, particle size distribution (PSD) of the API is a critical material attribute (CMA) for the tablet. In one embodiment of the tablets provided herein, particle size distribution of the API is selected to optimize the flow, blend homogeneity, and downstream tablet properties in a roller compaction process. In one embodiment, the particle size distribution of the API is optimized for manufacturability. In one embodiment, the particle size distribution of the API is optimized for manufacturability rather than dissolution.

In one embodiment of the tablets provided herein, particle size distribution (PSD) of the API is a critical material attribute (CMA) for the tablet. In one embodiment of the tablets provided herein, PSD of the API is selected to optimize the flow, blend homogeneity, and downstream tablet properties in a roller compaction process. In one embodiment, PSD of the API is optimized for manufacturability. In one embodiment, PSD of the API is optimized for manufacturability rather than dissolution.

The particle size can be measured by the techniques known in the art, including but not limited to laser diffraction, static image analysis, dynamic light scattering and sieve analysis. In one embodiment, the particle size is measured by laser diffraction. In one embodiment, the particle size is measured by dynamic light scattering.

In one embodiment, the API has particle size distribution such that d₁₀ is between about 1.00 μm to about 3.50 μm. In one embodiment, the API has particle size distribution such that d₁₀ is between about 1.50 μm to about 3.00 μm. In one embodiment, the API has particle size distribution such that d₁₀ is between about 1.50 μm to about 2.50 μm. In one embodiment, the API has particle size distribution such that d₁₀ is between about 1.50 μm to about 2.00 μm. In one embodiment, the API has particle size distribution such that d₁₀ is between about 1.61 μm to about 2.78 μm. In one embodiment, the API has particle size distribution such that d₁₀ is between about 2.22 μm to about 3.34 μm. In one embodiment, the API has particle size distribution such that d₁₀ is about 2.78 μm. In one embodiment, the API has particle size distribution such that d₁₀ is between about 1.76 μm to about 2.64 μm. In one embodiment, the API has particle size distribution such that d₁₀ is about 2.20 μm. In one embodiment, the API has particle size distribution such that d₁₀ is between about 1.31 μm to about 1.97 μm. In one embodiment, the API has particle size distribution such that d₁₀ is about 1.69 μm. In one embodiment, the API has particle size distribution such that d₁₀ is between about 1.29 μm to about 1.93 μm. In one embodiment, the API has particle size distribution such that d₁₀ is about 1.61 μm. In one embodiment, the API has particle size distribution such that d₁₀ is between about 1.39 μm to about 2.09 μm. In one embodiment, the API has particle size distribution such that d₁₀ is about 1.74 μm. In one embodiment, the API has particle size distribution such that d₁₀ is between about 1.56 μm to about 2.34 μm. In one embodiment, the API has particle size distribution such that d₁₀ is about 1.95 μm. In one embodiment, the API has particle size distribution such that d₁₀ is between about 1.58 μm to about 2.38 μm. In one embodiment, the API has particle size distribution such that d₁₀ is about 1.98 μm. In one embodiment, the API has particle size distribution such that d₁₀ is about 2.78 μm, about 2.20 μm, about 1.69 μm, about 1.61 μm, about 1.74 μm, about 1.95 μm or about 1.98 μm.

In one embodiment, the API has particle size distribution such that d₅₀ is between about 6.00 μm to about 20.00 μm. In one embodiment, the API has particle size distribution such that d₅₀ is between about 6.50 μm to about 18.00 μm. In one embodiment, the API has particle size distribution such that d₅₀ is between about 7.50 μm to about 15.00 μm. In one embodiment, the API has particle size distribution such that d₅₀ is between about 8.00 μm to about 12.00 μm. In one embodiment, the API has particle size distribution such that d₅₀ is between about 8 μm to about 14.50 μm. In one embodiment, the API has particle size distribution such that d₅₀ is between about 7.59 μm to about 14.46 μm. In one embodiment, the API has particle size distribution such that d₅₀ is between about 11.56 μm to about 17.35 μm. In one embodiment, the API has particle size distribution such that d₅₀ is about 14.46 μm. In one embodiment, the API has particle size distribution such that d₅₀ is between about 8.79 μm to about 13.19 μm. In one embodiment, the API has particle size distribution such that d₅₀ is about 19.99 μm. In one embodiment, the API has particle size distribution such that d₅₀ is between about 7.19 μm to about 10.79 μm. In one embodiment, the API has particle size distribution such that d₅₀ is about 8.99 μm. In one embodiment, the API has particle size distribution such that d₅₀ is between about 6.07 μm to about 9.11 μm. In one embodiment, the API has particle size distribution such that d₅₀ is about 7.59 μm. In one embodiment, the API has particle size distribution such that d₅₀ is between about 6.89 μm to about 10.34 μm. In one embodiment, the API has particle size distribution such that d₅₀ is about 8.62 μm. In one embodiment, the API has particle size distribution such that d₅₀ is between about 7.70 μm to about 11.56 μm. In one embodiment, the API has particle size distribution such that d₅₀ is about 9.63 μm. In one embodiment, the API has particle size distribution such that d₅₀ is between about 8.03 μm to about 12.04 μm. In one embodiment, the API has particle size distribution such that d₅₀ is about 10.04 μm. In one embodiment, the API has particle size distribution such that d₅₀ is about 14.46 μm, about 10.99 μm, about 8.99 μm, about 7.59 μm, about 8.62 μm, about 9.63 μm or about 10.04 μm.

In one embodiment, the API has particle size distribution such that d₉₀ is between about 20.00 μm to about 60.00 μm. In one embodiment, the API has particle size distribution such that d₉₀ is between about 25.00 μm to about 50.00 μm. In one embodiment, the API has particle size distribution such that d₉₀ is between about 30.00 μm to about 45.00 μm. In one embodiment, the API has particle size distribution such that d₉₀ is between about 25.00 μm to about 40.00 μm. In one embodiment, the API has particle size distribution such that d₉₀ is between about 27.74 μm to about 48.61 μm. In one embodiment, the API has particle size distribution such that d₉₀ is between about 38.89 μm to about 58.33 μm. In one embodiment, the API has particle size distribution such that d₉₀ is about 48.61 μm. In one embodiment, the API has particle size distribution such that d₉₀ is between about 31.87 μm to about 41.81 μm. In one embodiment, the API has particle size distribution such that d₉₀ is about 39.84 μm. In one embodiment, the API has particle size distribution such that d₉₀ is between about 25.56 μm to about 38.34 μm. In one embodiment, the API has particle size distribution such that d₉₀ is about 31.95 μm. In one embodiment, the API has particle size distribution such that d₉₀ is between about 22.19 μm to about 33.29 μm. In one embodiment, the API has particle size distribution such that d₉₀ is about 27.74 μm. In one embodiment, the API has particle size distribution such that d₉₀ is between about 23.64 μm to about 35.46 μm. In one embodiment, the API has particle size distribution such that d₉₀ is about 29.55 μm. In one embodiment, the API has particle size distribution such that d₉₀ is between about 30.75 μm to about 46.13 μm. In one embodiment, the API has particle size distribution such that d₉₀ is about 38.44 μm. In one embodiment, the API has particle size distribution such that d₉₀ is between about 28.82 μm to about 43.22 μm. In one embodiment, the API has particle size distribution such that d₉₀ is about 36.02 μm. In one embodiment, the API has particle size distribution such that d₉₀ is about 48.61 μm, about 39.84 μm, about 31.95 μm, about 27.74 μm, about 29.55 μm, about 38.44 μm or about 36.02 μm.

In one embodiment, the API has particle size distribution such that d₁₀ is between about 1.00 μm to about 3.50 μm, d₅₀ is between about 6.00 μm to about 20.00 μm and d₉₀ is between about 20.00 μm to about 60.00 μm. In one embodiment, the API has particle size distribution such that d₁₀ is between about 1.50 μm to about 3.00 μm, d₅₀ is between about 6.50 μm to about 18.00 μm and d₉₀ is between about 25.00 μm to about 50.00 μm. In one embodiment, the API has particle size distribution such that d₁₀ is between about 1.61 μm to about 2.78 μm, d₅₀ is between about 7.59 μm to about 14.46 μm and d₉₀ is between about 27.74 μm to about 48.61 μm. In one embodiment, the API has particle size distribution such that d₁₀ is about 2.78 μm, d₅₀ is about 14.46 μm and d₉₀ is between about 48.61 μm. In one embodiment, the API has particle size distribution such that d₁₀ is about 1.612.20 μm, d₅₀ is about 10.99 μm and d₉₀ is between about 39.84 μm. In one embodiment, the API has particle size distribution such that d₁₀ is about 1.69 μm, d₅₀ is about 8.99 μm and d₉₀ is between about 31.95 μm. In one embodiment, the API has particle size distribution such that d₁₀ is about 1.61 μm, d₅₀ is about 7.59 μm and d₉₀ is between about 27.74 μm. In one embodiment, the API has particle size distribution such that d₁₀ is about 1.74 μm, d₅₀ is about 8.62 μm and d₉₀ is between about 29.55 μm. In one embodiment, the API has particle size distribution such that d₁₀ is about 1.95 μm, d₅₀ is about 9.63 μm and d₉₀ is between about 38.44 μm.

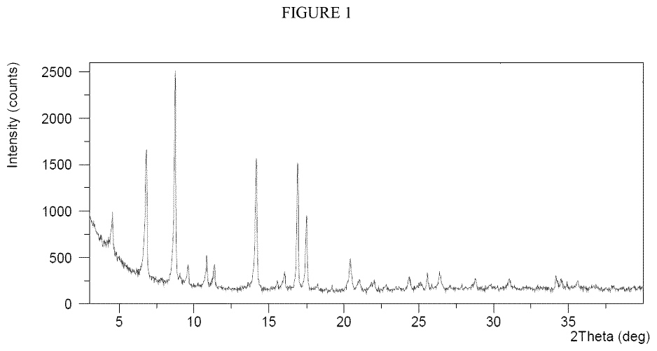

In certain embodiments, Form E used in the formulations provided herein is crystalline, as indicated by, e.g., X-ray powder diffraction measurements. In one embodiment, Form E of Compound 1 has an X-ray powder diffraction pattern substantially as shown in FIG. 1.

In one embodiment, Form E of Compound 1 has one or more characteristic X-ray powder diffraction peaks at a two-theta angle of approximately 8.64, 14.06, 16.81, 17.41, 25.47, 26.28, 34.07 or 34.39, degrees 2θ as depicted in FIG. 1. In another embodiment, Form E of Compound 1 has one, two, three, four or five characteristic X-ray powder diffraction peaks at a two-theta angle of approximately 8.64, 14.06, 16.81, 17.41 or 34.07 degrees 2θ. In another embodiment, Form E of Compound 1 has one, two, three or four characteristic X-ray powder diffraction peaks at a two-theta angle of approximately 8.64, 14.06, 16.81 or 17.41 degrees 2θ. In another embodiment, Form E of Compound 1 has one, two or three characteristic X-ray powder diffraction peaks at a two-theta angle of approximately 8.64, 16.81 or 17.41 degrees 2θ. In another embodiment, Form E of Compound 1 has one or more characteristic X-ray powder diffraction peaks as set forth in Table A. In another embodiment, Form E of Compound 1 has one, two, three, four, five, six or seven characteristic X-ray powder diffraction peaks as set forth in Table A. In another embodiment, Form E of Compound 1 has one, two, three, four or five characteristic X-ray powder diffraction peaks as set forth in Table A. In another embodiment, Form E of Compound 1 has one, two, or three characteristic X-ray powder diffraction peaks as set forth in Table A.

In one embodiment, Form E of Compound 1 has one or more characteristic X-ray powder diffraction peaks at a two-theta angle of approximately 16.81, 8.64, and 17.41 degrees 2θ as depicted in FIG. 1. In another embodiment, Form E of Compound 1 has one, two, three or four characteristic X-ray powder diffraction peaks at a two-theta angle of approximately 16.81, 8.64, and 17.41 or 14.06 degrees 2θ. In another embodiment, Form E of Compound 1 has one, two, three, four, five, six or seven characteristic X-ray powder diffraction peaks as set forth in Table A. In another embodiment, Form E of Compound 1 has one, two, or three characteristic X-ray powder diffraction peaks as set forth in Table A.

TABLE A

| Angle/° 2θ | Relative Intensity/% |
|---|---|
| 4.454 | 5.40 |
| 6.721 | 12.10 |
| 8.644 | 89.10 |
| 9.489 | 8.40 |
| 10.753 | 9.80 |
| 11.257 | 2.70 |
| 13.595 | 0.50 |
| 14.062 | 33.40 |
| 15.461 | 1.50 |
| 15.965 | 3.80 |
| 16.808 | 100.00 |
| 17.41 | 48.80 |
| 18.145 | 4.00 |
| 19.133 | 1.10 |
| 20.307 | 15.70 |
| 20.899 | 4.70 |
| 21.7 | 2.10 |
| 21.887 | 3.20 |
| 22.746 | 1.60 |
| 23.555 | 1.00 |
| 24.268 | 3.80 |
| 24.897 | 1.50 |
| 25.472 | 18.80 |
| 25.779 | 4.80 |
| 26.282 | 18.10 |
| 26.966 | 2.10 |
| 27.812 | 0.90 |
| 28.299 | 2.00 |
| 28.686 | 4.30 |
| 29.73 | 2.20 |
| 30.031 | 0.80 |
| 30.931 | 5.10 |
| 31.367 | 1.50 |
| 33.265 | 0.80 |
| 34.072 | 21.70 |
| 34.392 | 20.80 |
| 34.796 | 7.70 |
| 35.315 | 5.30 |
| 35.522 | 2.20 |
| 36.005 | 1.70 |
| 36.452 | 2.50 |
| 36.614 | 2.30 |
| 36.793 | 1.70 |
| 37.5 | 1.40 |
| 37.813 | 4.40 |
| 38.161 | 1.00 |
| 39.266 | 0.90 |
| 39.944 | 4.70 |
| 40.064 | 7.10 |
| 40.767 | 0.70 |
| 42.004 | 1.00 |
| 43.062 | 7.30 |
| 43.534 | 1.20 |
| 44.048 | 1.10 |
| 44.295 | 1.80 |
| 44.862 | 2.20 |
| 45.082 | 2.50 |
| 45.874 | 3.80 |
| 46.434 | 1.20 |
| 47.077 | 1.20 |
| 48.799 | 0.90 |
| 50.937 | 1.20 |
| 52.34 | 3.20 |
| 53.179 | 2.20 |
| 58.15 | 1.10 |

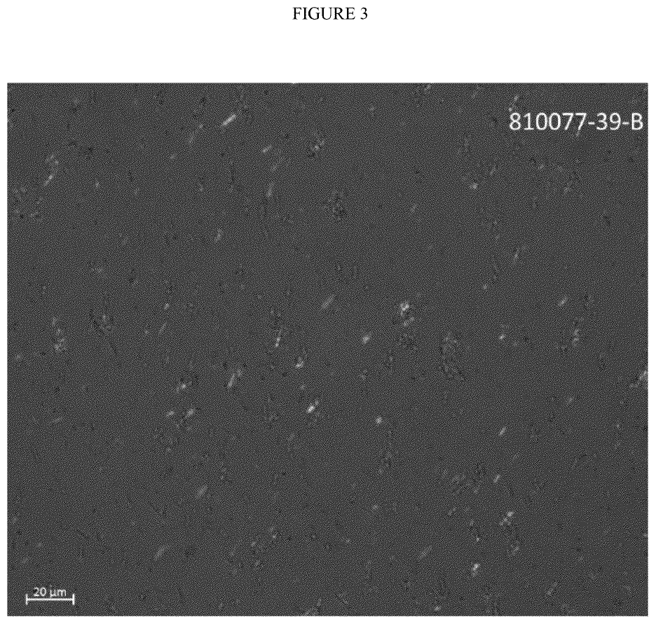

In one embodiment, provided herein is a crystalline form of Compound 1 having a thermogravimetric thermograph corresponding substantially to the representative TGA thermogram as depicted in FIG. 3. In certain embodiments, Form E shows a TGA weight loss of about 4.4% up to about 120° C. In certain embodiments, Form E shows a TGA weight loss of about 4.2% between about 120° C. and 220° C. In certain embodiments, Form E shows a two step TGA weight loss of about 4.4% up to about 120° C., and about 4.2% between about 120° C. and 220° C.

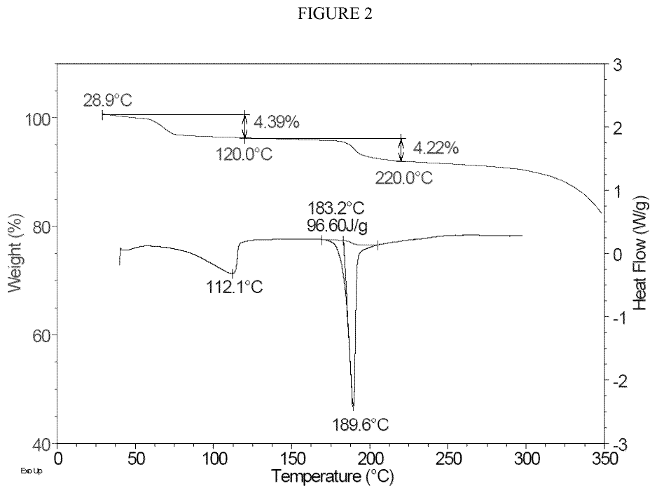

In one embodiment, provided herein is crystalline Form E of Compound 1 having a DSC thermogram corresponding substantially as depicted in FIG. 2. In certain embodiments, Form E is characterized by a DSC plot comprising two endotherms at 112.1° C. (peak) and 183.2° C. (onset).

In one embodiment, Form E of Compound 1 has the PLM picture as shown in FIG. 3.

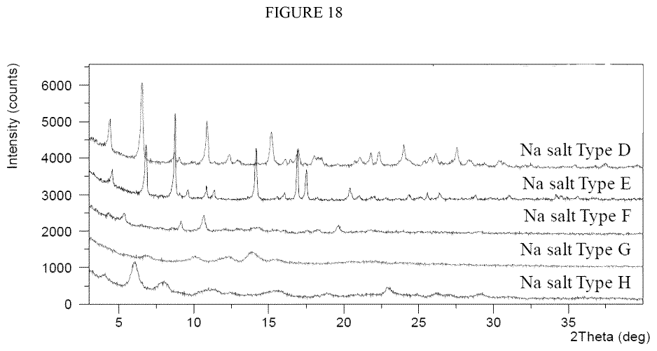

In certain embodiments, Form D used in the formulations provided herein is crystalline, as indicated by, e.g., X-ray powder diffraction measurements. In one embodiment, Form D of Compound 1 has an X-ray powder diffraction pattern substantially as shown in FIG. 18.

In one embodiment, Form D of Compound 1 has one or more characteristic X-ray powder diffraction peaks at a two-theta angle of approximately 4.41, 6.55, 10.88, 15.17, 24.01 or 27.54 degrees 2θ as depicted in FIG. 18. In another embodiment, Form D of Compound 1 has one, two, three, four or five characteristic X-ray powder diffraction peaks at a two-theta angle of approximately 4.41, 6.55, 10.88, 15.17, or 27.54 degrees 2θ. In another embodiment, Form D of Compound 1 has one, two, three or four characteristic X-ray powder diffraction peaks at a two-theta angle of approximately 4.41, 6.55, 10.88 or 15.17 degrees 2θ. In another embodiment, Form D of Compound 1 has one, two, or three characteristic X-ray powder diffraction peaks at a two-theta angle of approximately 6.55, 10.88 or 15.17 degrees 2θ. In another embodiment, Form D of Compound 1 has one or more characteristic X-ray powder diffraction peaks as set forth in Table 44. In another embodiment, Form D of Compound 1 has one, two, three, four, five, six or seven characteristic X-ray powder diffraction peaks as set forth in Table 44. In another embodiment, Form D of Compound 1 has one, two, or three characteristic X-ray powder diffraction peaks as set forth in Table 44.

TABLE 44
X-Ray Diffraction Peaks for Form D of Compound 1

| #Peak | 2θ [°] | d-spacing [Å] | Rel [%] |
|---|---|---|---|
| 1 | 4.41 | 20.00 | 38.28 |
| 2 | 6.55 | 13.48 | 100.00 |
| 3 | 8.72 | 10.13 | 12.87 |
| 4 | 9.03 | 9.78 | 7.08 |
| 5 | 10.10 | 8.78 | 1.91 |
| 6 | 10.88 | 8.12 | 53.65 |
| 7 | 12.38 | 7.15 | 12.72 |
| 8 | 12.96 | 6.83 | 5.44 |
| 9 | 15.17 | 5.84 | 44.45 |
| 10 | 16.09 | 5.50 | 7.68 |
| 11 | 16.42 | 5.39 | 7.08 |
| 12 | 16.80 | 5.26 | 10.96 |
| 13 | 16.97 | 5.22 | 17.16 |
| 14 | 17.43 | 5.08 | 2.65 |
| 15 | 18.01 | 4.92 | 13.03 |
| 16 | 18.30 | 4.84 | 10.76 |
| 17 | 18.51 | 4.79 | 10.99 |
| 18 | 19.58 | 4.53 | 3.67 |
| 19 | 20.75 | 4.28 | 5.27 |
| 20 | 21.03 | 4.22 | 10.67 |
| 21 | 21.82 | 4.07 | 16.23 |
| 22 | 22.34 | 3.98 | 18.78 |
| 23 | 24.01 | 3.70 | 22.86 |
| 24 | 24.34 | 3.65 | 6.83 |
| 25 | 25.36 | 3.51 | 6.13 |
| 26 | 25.72 | 3.46 | 10.63 |
| 27 | 26.09 | 3.41 | 15.24 |
| 28 | 27.24 | 3.27 | 3.84 |
| 29 | 27.54 | 3.24 | 24.15 |
| 30 | 28.37 | 3.14 | 8.07 |
| 31 | 28.90 | 3.09 | 2.12 |
| 32 | 29.40 | 3.03 | 3.58 |
| 33 | 30.36 | 2.94 | 8.60 |
| 34 | 30.71 | 2.91 | 6.92 |
| 35 | 32.47 | 2.75 | 6.26 |
| 36 | 35.54 | 2.52 | 3.16 |
| 37 | 37.48 | 2.40 | 5.46 |

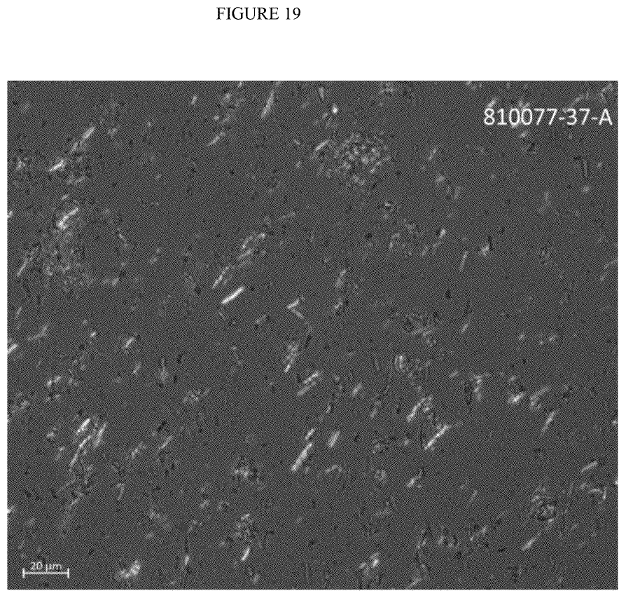

In one embodiment, Form D of Compound 1 has polarized light microscopic (PLM) picture as shown in FIG. 19.

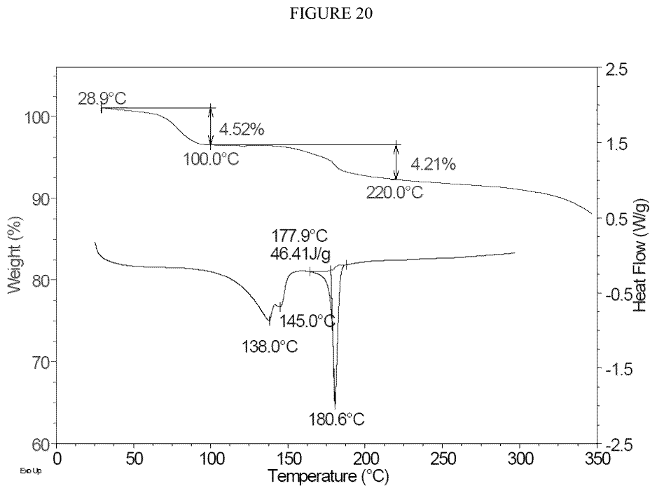

In one embodiment, Form D of Compound 1 has a thermogravimetric (TGA) thermograph corresponding substantially to the representative TGA thermogram as depicted in FIG. 20. In certain embodiments, Form D shows a TGA weight loss of about 6.5% up to about 100° C. In certain embodiments, Form D shows a TGA weight loss of about 4.5% between about 100° C. and 220° C. In certain embodiments, Form D shows a two-step TGA weight loss of about 6.5% up to about 100° C., and about 4.5% between about 100° C. and 220° C.

In one embodiment, Form D of Compound 1 has a DSC thermogram corresponding substantially as depicted in FIG. 20. In certain embodiments, Form D is characterized by a DSC plot comprising three endotherms at about 125.2° C., about 131.1° C. (peak) and about 177.5° C. (onset).

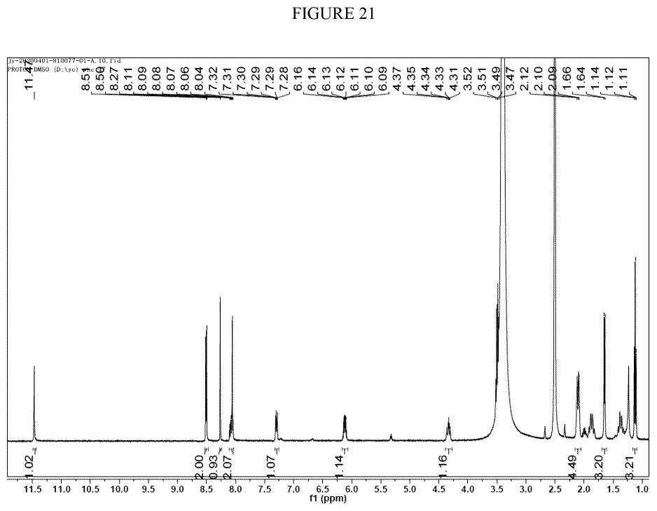

In one embodiment, Form D has ¹H NMR shown in FIG. 21.

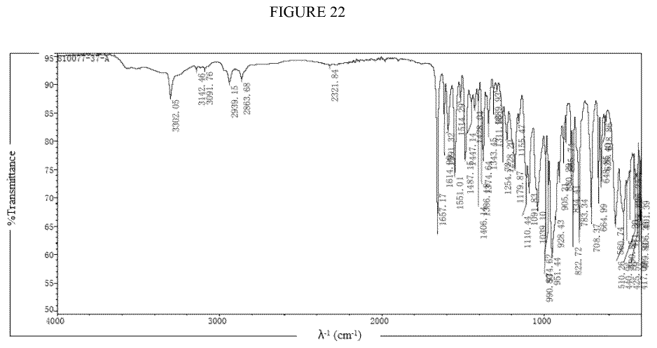

In one embodiment, Form D has FTIR spectrum provided in FIG. 22.

In certain embodiments, provided herein are tablet compositions comprising Compound 1 in about 15% to about 35% by weight based on total weight of the tablet. In certain embodiments, provided herein are tablet compositions comprising Compound 1 in about 20% to about 30% by weight based on total weight of the tablet. In certain embodiments, the tablet compositions provided herein comprise Compound 1 in about 22% to about 28% by weight based on total weight of the tablet. In certain embodiments, the tablet compositions provided herein comprise Compound 1 in about 20%, about 22%, about 24%, about 25%, about 25.67%, about 27%, or about 30% by weight based on total weight of the tablet. In certain embodiments, the tablet compositions provided herein comprise Compound 1 in an amount of about 25.67% by weight based on total weight of the tablet. In certain embodiments, the tablet compositions provided herein comprise Compound 1 in an amount of about 25% by weight based on total weight of the tablet.

In certain embodiments, provided herein are tablet compositions comprising 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in about 20% to about 30% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in about 22% to about 28% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in about 20%, about 22%, about 24%, about 25%, about 25.67%, about 26%, about 27%, or about 30% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in an amount of about 25.67% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in an amount of about 27% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in an amount of about 25% by weight based on total weight of the tablet.

In certain embodiments, provided herein are tablet compositions comprising Compound 1 in about 60% to about 85% by weight based on total weight of the tablet. In certain embodiments, the tablet compositions provided herein comprise Compound 1 in about 65% to about 75% by weight based on total weight of the tablet. In certain embodiments, the tablet compositions provided herein comprise Compound 1 in about 60% to about 75% by weight based on total weight of the tablet. In certain embodiments, the tablet compositions provided herein comprise Compound 1 in about 65% to about 75% by weight based on total weight of the tablet. In certain embodiments, the tablet compositions provided herein comprise Compound 1 in about 65%, about 68%, about 70%, about 72% or about 75% by weight based on total weight of the tablet. In certain embodiments, the tablet compositions provided herein comprise Compound 1 in an amount of about 70% by weight based on total weight of the tablet. In certain embodiments, the tablet compositions provided herein comprise Compound 1 in an amount of about 64.96% by weight based on total weight of the tablet.

In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in about 60% to about 80% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in about 65% to about 85% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in about 65% to about 75% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in about 65%, about 68%, about 70%, about 72% or about 75% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in an amount of about 70% by weight based on total weight of the tablet. In certain embodiments, the tablet compositions provided herein comprise 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt in an amount of about 64.96% by weight based on total weight of the tablet.

In certain embodiments, provided herein is a tablet comprising about 150 mg, about 250 mg, about 500 mg, about 750 mg or about 1000 mg of Compound 1. In certain embodiments, provided herein is a tablet comprising about 150 mg, about 250 mg, about 500 mg, about 750 mg or about 1000 mg of Compound 1A. In certain embodiments, the tablets provided herein are tablets of about 150 mg, about 250 mg, about 500 mg, about 750 mg or 1000 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In certain embodiments, the tablets provided herein are tablets of about 150 mg, about 250 mg, about 500 mg, about 750 mg or 1000 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

In certain embodiments, provided herein is a tablet comprising about 150 mg or about 250 mg of Compound 1. In certain embodiments, provided herein is a tablet comprising about 150 mg or about 250 mg of Compound 1A. In certain embodiments, the tablets provided herein are tablets of about 150 mg or about 250 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In certain embodiments, the tablets provided herein are tablets of about 150 mg or about 250 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

The tablets provided herein further comprise one or more intragranular and extragranular excipients.

In certain embodiments, the particle size of the excipients used in the formulations provided herein is selected such that a favorable size ratio between the excipients and the API particles is achieved to allow the fine API to adhere to or intersperse among larger, free flowing excipient particles, thereby improving percolation behavior and reducing both sifting and fluidization segregation mechanisms during transfer, hopper discharge, and die filling.

### 2. Intragranular Excipients

In one embodiment, the tablets provided herein comprise one or more intragranular excipients selected from the group consisting of a diluent, a disintegrant, a glidant and a lubricant. In one embodiment, the tablets provided herein comprise the intra-granular excipients from about 50% to about 70% by weight based on total weight of the tablet. In one embodiment, the tablets provided herein comprise the intra-granular excipients from about 55% to about 65% by weight based on total weight of the tablet. In one embodiment, the tablets provided herein comprise the intra-granular excipients from about 60%, about 61%, about 62%, about 62.5%, 63%, about 64%, about 65% or about 70% by weight based on total weight of the tablet. In one embodiment, the tablets provided herein comprise the intra-granular excipients from about 62%, about 62.5% or about 63% by weight based on total weight of the tablet.

In one embodiment, the tablets provided herein comprise the intra-granular excipients from about 15% to about 25% by weight based on total weight of the tablet. In one embodiment, the tablets provided herein comprise the intra-granular excipients from about 15% to about 20% by weight based on total weight of the tablet. In one embodiment, the tablets provided herein comprise the intra-granular excipients from about 15%, about 17%, about 20%, about 22%, about 23%, about 24%, or about 25% by weight based on total weight of the tablet. In one embodiment, the tablets provided herein comprise the intra-granular excipients from about 17% by weight based on total weight of the tablet. In one embodiment, the tablets provided herein comprise the intra-granular excipients from about 22% by weight based on total weight of the tablet.

### (a) Diluents

Diluents are excipients which are used for diluting formulation components such as active ingredients and adjusting them to amounts appropriate to the formulation, and in some cases, for imparting stability, improved moldability, and the like. Diluents are also referred to as fillers or bulking agents.

In certain embodiments, the pharmaceutical compositions provided herein comprise a ductile diluent and/or a brittle diluent. In certain embodiments, the pharmaceutical compositions provided herein comprise two diluents, wherein one diluent is a ductile diluent and the other diluent is a brittle diluent. In certain embodiments, the pharmaceutical compositions provided herein comprise a water soluble diluent and/or a water insoluble diluent. In certain embodiments, the pharmaceutical compositions provided herein comprise two diluents, wherein one diluent is a water soluble diluent and the other diluent is a water insoluble diluent.

Examples of diluents include lactose, glucose, sucrose, maltose (preferably candy powder (containing 83% or more of maltose)), trehalose, sugars such as lactose and fructose, sugar alcohols such as mannitol, xylitol, maltitol, sorbitol, and erythritol, and crystalline cellulose. In one embodiment, the diluent used in the tablets provided herein is microcrystalline cellulose. In one embodiment, the diluent used in the tablets provided herein is mannitol. In one embodiment, the diluent used in the tablets provided herein is a combination of microcrystalline cellulose and mannitol.

Without being limited by theory, the use of two diluents (e.g., microcrystalline cellulose and mannitol) is thought to have a synergistic effect on the formulations provided herein, as microcrystalline cellulose (e.g., AVICEL®) is a ductile diluent, while mannitol (e.g., PARTECK®) is brittle. The brittle diluent is thought to help prevent relaxation after compression of the formulations provided herein. Prototype experimental runs using AVICEL® and PARTECK® as diluents showed that a combination of the two diluents provides improved disintegration times, which is thought to be because AVICEL® is not water soluble and PARTECK® is water soluble. Without being limited by theory, it is thought that using a single diluent could potentially impact disintegration and likely dissolution, and also could change the compression characteristics.

In certain embodiments, the intragranular excipients in the tablet comprise a diluent in an amount of about 55% to about 65% by weight based on the total weight of the tablet. In certain embodiments, the intragranular excipients in the tablet comprise a diluent in an amount of about 57% to about 63% by weight based on the total weight of the tablet. In certain embodiments, the intragranular excipients in the tablet comprise a diluent in an amount of about 57% to about 58% by weight based on the total weight of the tablet. In certain embodiments, the intragranular excipients in the tablet comprise a diluent in an amount of about 58% to about 59% by weight based on the total weight of the tablet. In certain embodiments, the intragranular excipients in the tablet comprise a diluent in an amount of about 55%, about 56%, about 57%, about 57.8%, about 58%, about 58.5%, about 59%, about 60%, about 62%, or about 63% or about 65% by weight based on the total weight of the tablet. In certain embodiments, the intragranular excipients in the tablet comprise a diluent in an amount of about 57%, about 57.8%, about 58%, about 58.5% or about 59% by weight based on the total weight of the tablet.

In certain embodiments, the tablet comprises a diluent in an amount of about 4% to about 20% by weight based on the total weight of the tablet. In certain embodiments, the tablet comprises a diluent in an amount of about 6% to about 20% by weight based on the total weight of the tablet. In certain embodiments, the tablet comprises a diluent in an amount of about 10% to about 20% by weight based on the total weight of the tablet. In certain embodiments, the tablet comprises a diluent in an amount of about 11% to about 13% by weight based on the total weight of the tablet. In certain embodiments, the tablet comprises a diluent in an amount of about 16% to about 18% by weight based on the total weight of the tablet. In certain embodiments, the tablet comprises a diluent in an amount of about 4%, about 6%, about 8%, about 10%, about 12%, about 15%, about 17%, about 19% or about 20% by weight based on the total weight of the tablet. In certain embodiments, the tablet comprises a diluent in an amount of about 12% by weight based on the total weight of the tablet. In certain embodiments, the tablet comprises a diluent in an amount of about 17% by weight based on the total weight of the tablet.

In certain embodiments, the intragranular excipients in the tablet comprise microcrystalline cellulose in an amount of about 25% to about 35% by weight based on the total weight of the tablet, such as for example about 27% to about 33% by weight based on the total weight of the tablet. In certain embodiments, the intragranular excipients in the tablet comprise microcrystalline cellulose in an amount of about 28%, about 28.92%, about 29%, about 29.25%, about 30% or about 32% by weight based on the total weight of the tablet. In certain embodiments, the intragranular excipients in the tablet comprise microcrystalline cellulose in an amount of about 28.92% by weight based on the total weight of the tablet. In certain embodiments, the intragranular excipients in the tablet comprise microcrystalline cellulose in an amount of about 29.25% by weight based on the total weight of the tablet.

In certain embodiments, the diluent comprises microcrystalline cellulose in an amount of about 4 to about 10% by weight based on the total weight of the tablet, such as for example about 5% to about 9% by weight based on the total weight of the tablet. In certain embodiments, the diluent comprises microcrystalline cellulose in an amount of about 4%, about 5%, about 6%, about 7%, about 8%, about 8.5% or about 9% by weight based on the total weight of the tablet. In certain embodiments, the diluent comprises microcrystalline cellulose in an amount of about 6% by weight based on the total weight of the tablet. In certain embodiments, the diluent comprises microcrystalline cellulose in an amount of about 8.5% by weight based on the total weight of the tablet.

In certain embodiments, the intragranular excipients in the tablet comprise mannitol in an amount of about 25% to about 35% by weight based on the total weight of the tablet, such as for example about 27% to about 33% by weight based on the total weight of the tablet. In certain embodiments, the intragranular excipients in the tablet comprise mannitol in an amount of about 28%, about 28.92%, about 29%, about 29.25%, about 30% or about 32% by weight based on the total weight of the tablet. In certain embodiments, the intragranular excipients in the tablet comprise mannitol in an amount of about 28.92% by weight based on the total weight of the tablet. In certain embodiments, the intragranular excipients in the tablet comprise mannitol in an amount of about 29.25% by weight based on the total weight of the tablet.

In certain embodiments, the diluent comprises mannitol in an amount of about 4 to about 10% by weight based on the total weight of the tablet, such as for example about 5% to about 9% by weight based on the total weight of the tablet. In certain embodiments, the diluent comprises mannitol in an amount of about 4%, about 5%, about 6%, about 7%, about 8%, about 8.5%, about 9% by weight based on the total weight of the tablet. In certain embodiments, the diluent comprises mannitol in an amount of about 6% by weight based on the total weight of the tablet. In certain embodiments, the diluent comprises mannitol in an amount of about 8.5% by weight based on the total weight of the tablet. In one embodiment, mannitol has a medium to large particle size. In certain embodiments, the particle size for mannitol is about 100 μm to 200 μm. In certain embodiments, the particle size for mannitol is about 100 μm. In certain embodiments, the intragranular excipients in the tablet comprise microcrystalline cellulose and mannitol each in an amount of about 25% to about 35% by weight based on the total weight of the tablet, such as for example about 27% to about 33% by weight based on the total weight of the tablet. In certain embodiments, the intragranular excipients in the tablet comprise microcrystalline cellulose and mannitol each in an amount of about 28%, about 28.92%, about 29%, about 29.25%, about 30% or about 32% by weight based on the total weight of the tablet. In certain embodiments, the intragranular excipients in the tablet comprise microcrystalline cellulose and mannitol in equal amounts of about 28%, about 28.92%, about 29%, about 29.25%, about 30% or about 32% by weight based on the total weight of the tablet. In certain embodiments, the intragranular excipients in the tablet comprise microcrystalline cellulose and mannitol each in an amount of about 28.92% by weight based on the total weight of the tablet. In certain embodiments, the intragranular excipients in the tablet comprise microcrystalline cellulose and mannitol each in an amount of about 29.25% by weight based on the total weight of the tablet. In one embodiment, mannitol and microcrystalline cellulose has a medium to large particle size. In certain embodiments, the particle size for mannitol and microcrystalline cellulose is about 100 μm to about 200 μm. In certain embodiments, the particle size for mannitol and microcrystalline cellulose is about 100 μm.

In certain embodiments, the diluent comprises microcrystalline cellulose and mannitol each in an amount of about 4 to about 9% by weight based on the total weight of the tablet, such as for example about 6% to about 9% by weight based on the total weight of the tablet. In certain embodiments, the diluent comprises microcrystalline cellulose and mannitol each in an amount of about 4%, about 5%, about 6%, about 7%, about 8%, about 8.5% or about 9% by weight based on the total weight of the tablet. In certain embodiments, the diluent comprises microcrystalline cellulose and mannitol each in an amount of about 8.5% by weight based on the total weight of the tablet. In certain embodiments, the diluent comprises microcrystalline cellulose and mannitol each in an amount of about 6% by weight based on the total weight of the tablet.

### (b) Disintegrants

Disintegrants are excipients to improve the disintegration of a preparation, more particularly, they are excipients to be added to disintegrate a tablet by absorbing water in the body after administration, swelling, and thereby facilitating release of the active ingredient. In certain embodiments, the amount of disintegrators in the tablets provided herein is selected such that the disintegration and the dissolution of the tablet are not reduced.

Examples of the disintegrants include: sodium starch glycolate (product name: Primojel, GLYCOLYS, EXPLOTAB, and the like), sodium alginate, carmellose, croscarmellose, croscarmellose calcium, and croscarmellose sodium, glycerin fatty acid ester, low-substituted sodium carboxymethyl starch and partially pregelatinized starch (product name: LYCATAB C, PCS, Graflow, starch 1500, and the like).

In one embodiment, the disintegrant in the tablet provided herein is croscarmellose sodium.

In one embodiment, the intragranular excipients in the tablet comprise a disintegrant in about 2% to about 4% by weight based on the total weight of the tablet. In one embodiment, the tablet comprises a disintegrant in about 2.5%, about 3% or about 4% by weight based on the total weight of the tablet. In one embodiment, the intragranular excipients in the tablet comprise a disintegrant in about 3% by weight based on the total weight of the tablet.

In one embodiment, the intragranular excipients in the tablet comprise croscarmellose sodium in the amount of about 2% to about 4% by weight based on the total weight of the tablet. In one embodiment, the intragranular excipients in the tablet comprise croscarmellose sodium in the amount of about 2.5%, about 3% or about 4% by weight based on the total weight of the tablet. In one embodiment, the intragranular excipients in the tablet comprise croscarmellose sodium in the amount of about 3% by weight based on the total weight of the tablet.

### (c) Glidants

In one embodiment, the glidant used in the tablets provided herein is fumed silica or colloidal silicon dioxide. In one embodiment, the glidant used in the tablets provided herein is fumed silica. In certain embodiments, the intragranular excipients in the tablet comprise a glidant in an amount of about 0.2% to about 2% by weight based on the total weight of the tablet. In certain embodiments, the intragranular excipients in the tablet comprise fumed silica which is present in an amount of about 0.5%, about 1% or about 2% by weight based on the total weight of the tablet. In certain embodiments, the intragranular excipients in the tablet comprise fumed silica which is present in an amount of about 1% by weight based on the total weight of the tablet. In certain embodiments, the intragranular excipients in the tablet comprise fumed silica which is present in an amount of about 0.5% by weight based on the total weight of the tablet.

### (d) Lubricants

Examples of lubricants include magnesium stearate, calcium stearate, sucrose fatty acid ester, polyethylene glycol, talc, sodium stearyl fumarate, and stearic acid. In one embodiment, the lubricant used in the tablet formulations herein is sodium stearyl fumarate.

In one embodiment, the intragranular excipients in the tablet comprise sodium stearyl fumarate in an amount of about 0.5% to about 2% by weight based on the total weight of the tablet. In one embodiment, the intragranular excipients in the tablet comprise sodium stearyl fumarate in an amount of about 0.5%, about 1% or about 2% by weight based on the total weight of the tablet. In one embodiment, the intragranular excipients in the tablet comprise sodium stearyl fumarate in an amount of about 1% by weight based on the total weight of the tablet.

### (e) Other Excipients

The tablet provided herein may contain various excipients other than the above-mentioned excipients, which are pharmaceutically acceptable and used as excipients. Examples of the other excipients include, but are not limited to, solubility enhancers, stabilizers, pH adjustors, coating agents and pigments. In one embodiment, the other excipient is selected from a solubility enhancer, a stabilizer, and a pH adjustor.

The amount of these excipients in the tablets provided herein is selected such that the dissolution of Compound 1 from the tablet is not negatively affected. In one embodiment, the total amount of these excipients is about 5% or less by weight based on the total weight of the tablet or in one embodiment, the amount is 3% or less by weight of the tablet or in one embodiment, the amount is 1% or less by weight of the tablet.

In certain embodiments, the tablets comprise a pigment.

### 3. Extragranular Excipients

In one embodiment, the tablets provided herein comprise one or more extragranular excipients selected from the group consisting of a compression aid, a disintegrant, a glidant and a lubricant. In one embodiment, the tablets provided herein comprise the extra-granular excipients from about 10% to about 20% by weight based on total weight of the tablet. In one embodiment, the tablets provided herein comprise the extra-granular excipients in about 10%, about 11%, about 12%, about 13%, about 14%, about 15%, about 16%, about 17%, about 18%, about 19% or about 20% by weight based on total weight of the tablet. In one embodiment, the tablets provided herein comprise the extra-granular excipients in about 12% by weight based on total weight of the tablet. In one embodiment, the tablets provided herein comprise the extra-granular excipients in about 13% by weight based on total weight of the tablet.

In one embodiment, the tablets provided herein comprise one or more extragranular excipients selected from the group consisting of a compression aid, a disintegrant, a glidant and a lubricant.

### (a) Compression Aid

In one embodiment, the compression aid used in the extragranular excipient is microcrystalline cellulose. In certain embodiments, the extragranular excipients in the tablet comprise a compression aid in an amount of about 6% to about 10% by weight based on the total weight of the tablet. In certain embodiments, the extragranular excipients in the tablet comprise a compression aid in an amount of about 6%, about 7%, about 8% or about 10% by weight based on the total weight of the tablet. In certain embodiments, the extragranular excipients in the tablet comprise a compression aid in an amount of about 8% by weight based on the total weight of the tablet. In certain embodiments, the extragranular excipients in the tablet comprise a compression aid in an amount of about 7% by weight based on the total weight of the tablet.

In certain embodiments, the extragranular excipients in the tablet comprise microcrystalline cellulose in an amount of about 6% to about 10% by weight based on the total weight of the tablet. In certain embodiments, the extragranular excipients in the tablet comprise microcrystalline cellulose in an amount of about 6%, about 7%, about 8% or about 10% by weight based on the total weight of the tablet. In certain embodiments, the extragranular excipients in the tablet comprise microcrystalline cellulose in an amount of about 7% by weight based on the total weight of the tablet. In certain embodiments, the extragranular excipients in the tablet comprise microcrystalline cellulose in an amount of about 8% by weight based on the total weight of the tablet.

### (b) Disintegrants

In one embodiment, the disintegrant used in the extragranular excipient is croscarmellose sodium. In certain embodiments, the extragranular excipients in the tablet comprise croscarmellose sodium in an amount of about 2% to about 4% by weight based on the total weight of the tablet. In certain embodiments, the extragranular excipients in the tablet comprise croscarmellose sodium in an amount of about 2.5%, about 3% or about 4% by weight based on the total weight of the tablet. In certain embodiments, the extragranular excipients in the tablet comprise croscarmellose sodium in an amount of about 3% by weight based on the total weight of the tablet.

### (c) Lubricants

In one embodiment, the lubricant used in the extragranular excipient is sodium stearyl fumarate. In certain embodiments, the extragranular excipients in the tablet comprise sodium stearyl fumarate in an amount of about 1% to about 3% by weight based on the total weight of the tablet. In certain embodiments, the extragranular excipients in the tablet comprise sodium stearyl fumarate in an amount of about 1%, about 2% or about 3% by weight based on the total weight of the tablet. In certain embodiments, the extragranular excipients in the tablet comprise sodium stearyl fumarate in an amount of about 2% by weight based on the total weight of the tablet.

### (d) Other Excipients

The tablet provided herein may contain various extragranular excipients other than the above-mentioned excipients, which are pharmaceutically acceptable and used as excipients. Examples of the other excipients include, but are not limited to, coloring agents, coating agents and flavoring agents.

### 4. Exemplary Tablet Formulations

The tablets provided herein comprise a combination of the API and the pharmaceutically acceptable excipients in the relative proportions as described. The API and the pharmaceutically acceptable excipients are combined in the manner described. Exemplary tablet formulations are described herein.

### A. Low Drug Load Tablet Formulations

Examples of the tablets provided herein having the desired stability during storage include a tablet comprising:
a) about 15% to about 35% Compound 1 by weight based on total weight of the tablet;
b) about 50% to about 70% intra-granular excipient by weight based on total weight of the tablet; and
c) about 10% to about 20% extra-granular excipient by weight based on total weight of the tablet.

In one embodiment, the tablet provided herein comprises:
a) about 20% to about 30% Compound 1 by weight based on total weight of the tablet;
b) an intra-granular excipient comprising about 25% to about 35% microcrystalline cellulose by weight based on total weight of the tablet, about 25% to about 35% mannitol by weight based on total weight of the tablet, about 2% to about 4% croscarmellose sodium by weight based on total weight of the tablet, about 0.2% to about 2% fumed silica by weight based on total weight of the tablet, and about 0.5% to about 2% sodium stearyl fumarate by weight based on total weight of the tablet; and
c) an extra-granular excipient comprising about 6% to about 10% microcrystalline cellulose by weight based on total weight of the tablet, about 2% to about 4% croscarmellose sodium by weight based on total weight of the tablet, and about 1% to about 3% sodium stearyl fumarate by weight based on total weight of the tablet.

In one embodiment, the tablets provided herein having the desired stability during storage include a tablet comprising:
a) about 25% Compound 1 by weight based on total weight of the tablet;
b) the intra-granular excipient comprising about 29% microcrystalline cellulose by weight based on total weight of the tablet, about 29% mannitol by weight based on total weight of the tablet, about 3% croscarmellose sodium by weight based on total weight of the tablet, about 0.5% fumed silica by weight based on total weight of the tablet, and about 1% sodium stearyl fumarate by weight based on total weight of the tablet; and
c) the extra-granular excipient comprising about 7% microcrystalline cellulose by weight based on total weight of the tablet, about 3% croscarmellose sodium by weight based on total weight of the tablet, and about 2% sodium stearyl fumarate by weight based on total weight of the tablet.

In one embodiment, the tablets provided herein having the desired stability during storage include a tablet comprising:
a) about 25.7% Compound 1 by weight based on total weight of the tablet;
b) the intra-granular excipient comprising about 29% microcrystalline cellulose by weight based on total weight of the tablet, about 29% mannitol by weight based on total weight of the tablet, about 3% croscarmellose sodium by weight based on total weight of the tablet, about 0.5% fumed silica by weight based on total weight of the tablet, and about 1% sodium stearyl fumarate by weight based on total weight of the tablet; and
c) the extra-granular excipient comprising about 7% microcrystalline cellulose by weight based on total weight of the tablet, about 3% croscarmellose sodium by weight based on total weight of the tablet, and about 2% sodium stearyl fumarate by weight based on total weight of the tablet.

In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium. In one embodiment, Compound 1 is solid Form E of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium.

In one embodiment, the tablet comprises:
a) about 250.00 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 292.50 mg microcrystalline cellulose, about 292.50 mg mannitol, about 30 mg croscarmellose sodium, about 5 mg fumed silica, and about 10 mg sodium stearyl fumarate; and
c) the extra-granular excipient comprising 70 mg microcrystalline cellulose, about 30 mg croscarmellose sodium, and about 20 mg sodium stearyl fumarate.

In one embodiment, the tablet comprises:
a) about 269.39 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 283.20 mg microcrystalline cellulose, about 282.50 mg mannitol, about 30 mg croscarmellose sodium, about 5 mg fumed silica, and about 10 mg sodium stearyl fumarate; and
c) the extra-granular excipient comprising 70 mg microcrystalline cellulose, about 30 mg croscarmellose sodium, and about 20 mg sodium stearyl fumarate.

In one embodiment, the tablet comprises:
a) about 150 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 175.50 mg microcrystalline cellulose, about 175.50 mg mannitol, about 18 mg croscarmellose sodium, about 3 mg fumed silica, and about 6 mg sodium stearyl fumarate; and
c) the extra-granular excipient comprising 42 mg microcrystalline cellulose, about 18 mg croscarmellose sodium, and about 12 mg sodium stearyl fumarate.

In one embodiment, the tablet comprises:
a) about 154 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 173.47 mg microcrystalline cellulose, about 173.47 mg mannitol, about 18 mg croscarmellose sodium, about 3 mg fumed silica, and about 6 mg sodium stearyl fumarate; and
c) the extra-granular excipient comprising 42 mg microcrystalline cellulose, about 18 mg croscarmellose sodium, and about 12 mg sodium stearyl fumarate.

### B. High Drug Load Tablet Formulations

In one embodiment, the tablet provided herein comprises:
a) about 60% to about 85% Compound 1 by weight based on total weight of the tablet;
b) about 15% to about 25% intra-granular excipient by weight based on total weight of the tablet; and
c) about 10% to about 20% extra-granular excipient by weight based on total weight of the tablet.

In one embodiment, the tablet provided herein comprises:
a) about 65% to about 85% Compound 1 by weight based on total weight of the tablet;
b) about 15% to about 20% intra-granular excipient by weight based on total weight of the tablet; and
c) about 10% to about 15% extra-granular excipient by weight based on total weight of the tablet.

In certain embodiments, the tablets provided herein having the desired stability during storage include a tablet comprises:
a) about 60% to about 75% Compound 1 by weight based on total weight of the tablet;
b) an intra-granular excipient comprising about 4% to about 10% microcrystalline cellulose by weight based on total weight of the tablet, about 4% to about 10% mannitol by weight based on total weight of the tablet, about 2% to about 4% croscarmellose sodium by weight based on total weight of the tablet, about 0.2% to about 2% fumed silica by weight based on total weight of the tablet, and about 0.5% to about 2% sodium stearyl fumarate by weight based on total weight of the tablet; and
c) an extra-granular excipient comprising about 6% to about 10% microcrystalline cellulose by weight based on total weight of the tablet, about 2% to about 4% croscarmellose sodium by weight based on total weight of the tablet, and about 1% to about 3% sodium stearyl fumarate by weight based on total weight of the tablet.

In one embodiment, the tablets provided herein having the desired stability during storage include a tablet comprising:
a) about 65% Compound 1 by weight based on total weight of the tablet;
b) the intra-granular excipient comprising about 8.5% microcrystalline cellulose by weight based on total weight of the tablet, about 8.5% mannitol by weight based on total weight of the tablet, about 3% croscarmellose sodium by weight based on total weight of the tablet, about 1% fumed silica by weight based on total weight of the tablet, and about 1% sodium stearyl fumarate by weight based on total weight of the tablet; and
c) the extra-granular excipient comprising about 8% microcrystalline cellulose by weight based on total weight of the tablet, about 3% croscarmellose sodium by weight based on total weight of the tablet, and about 2% sodium stearyl fumarate by weight based on total weight of the tablet.

In one embodiment, the tablets provided herein having the desired stability during storage include a tablet comprising:
a) about 70% Compound 1 by weight based on total weight of the tablet;
b) the intra-granular excipient comprising about 6% microcrystalline cellulose by weight based on total weight of the tablet, about 6% mannitol by weight based on total weight of the tablet, about 3% croscarmellose sodium by weight based on total weight of the tablet, about 1% fumed silica by weight based on total weight of the tablet, and about 1% sodium stearyl fumarate by weight based on total weight of the tablet; and
c) the extra-granular excipient comprising about 8% microcrystalline cellulose by weight based on total weight of the tablet, about 3% croscarmellose sodium by weight based on total weight of the tablet, and about 2% sodium stearyl fumarate by weight based on total weight of the tablet.

In one embodiment, the tablet comprises:
a) about 269.40 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 23.09 mg microcrystalline cellulose, about 23.09 mg mannitol, about 11.55 mg croscarmellose sodium, about 3.85 mg fumed silica, and about 3.85 mg sodium stearyl fumarate; and
c) the extra-granular excipient comprising 30.79 mg microcrystalline cellulose, about 11.55 mg croscarmellose sodium, and about 7.70 mg sodium stearyl fumarate.

In one embodiment, the tablet comprises:
a) about 250 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 32.79 mg microcrystalline cellulose, about 32.79 mg mannitol, about 11.55 mg croscarmellose sodium, about 3.85 mg fumed silica, and about 3.85 mg sodium stearyl fumarate; and
c) the extra-granular excipient comprising 30.79 mg microcrystalline cellulose, about 11.55 mg croscarmellose sodium, and about 7.70 mg sodium stearyl fumarate.

In one embodiment, the tablet comprises:
a) about 538.79 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt;
b) the intra-granular excipients comprising about 46.18 mg microcrystalline cellulose, about 46.18 mg mannitol, about 23.09 mg croscarmellose sodium, about 7.70 mg fumed silica, and about 7.70 mg sodium stearyl fumarate; and
c) the extra-granular excipient comprising about 61.58 mg microcrystalline cellulose, about 23.09 mg croscarmellose sodium, and about 15.39 mg sodium stearyl fumarate.

In one embodiment, the tablet comprises:
a) about 500 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt;
b) the intra-granular excipients comprising about 65.58 mg microcrystalline cellulose, about 65.58 mg mannitol, about 23.09 mg croscarmellose sodium, about 7.70 mg fumed silica, and about 7.70 mg sodium stearyl fumarate; and
c) the extra-granular excipient comprising about 61.58 mg microcrystalline cellulose, about 23.09 mg croscarmellose sodium, and about 15.39 mg sodium stearyl fumarate.

In one embodiment, the tablet comprises:
a) about 808.19 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt;
b) the intra-granular excipients comprising about 69.27 mg microcrystalline cellulose, about 69.27 mg mannitol, about 34.64 mg croscarmellose sodium, about 11.55 mg fumed silica, and about 11.55 mg sodium stearyl fumarate; and
c) the extra-granular excipient comprising about 92.36 mg microcrystalline cellulose, about 34.64 mg croscarmellose sodium, and about 23.09 mg sodium stearyl fumarate.

In one embodiment, the tablet comprises:
a) about 750 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt;
b) the intra-granular excipients comprising about 98.37 mg microcrystalline cellulose, about 98.37 mg mannitol, about 34.64 mg croscarmellose sodium, about 11.55 mg fumed silica, and about 11.55 mg sodium stearyl fumarate; and
c) the extra-granular excipient comprising about 92.36 mg microcrystalline cellulose, about 34.64 mg croscarmellose sodium, and about 23.09 mg sodium stearyl fumarate.

In one embodiment, the tablet comprises:
a) about 1077.58 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt;
b) the intra-granular excipients comprising about 92.37 mg microcrystalline cellulose, about 92.37 mg mannitol, about 46.18 mg croscarmellose sodium, about 15.39 mg fumed silica, and about 15.39 mg sodium stearyl fumarate; and
c) the extra-granular excipient comprising about 123.15 mg microcrystalline cellulose, about 46.18 mg croscarmellose sodium, and about 30.79 mg sodium stearyl fumarate.

In one embodiment, the tablet comprises:
a) about 999.99 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt;
b) the intra-granular excipients comprising about 131.16 mg microcrystalline cellulose, about 131.16 mg mannitol, about 46.18 mg croscarmellose sodium, about 15.39 mg fumed silica, and about 15.39 mg sodium stearyl fumarate; and
c) the extra-granular excipient comprising about 123.15 mg microcrystalline cellulose, about 46.18 mg croscarmellose sodium, and about 30.79 mg sodium stearyl fumarate.

Tables B and C below provide compositions for exemplary low drug load and Tables D and E below provide compositions for exemplary high drug load tablet formulations.

TABLE B
Low drug load tablet formulations

| | | | Formulation | |
|---|---|---|---|---|
| | | Unit | 150 mg | 250 mg |
| | | Composition | Compound 1A | Compound 1A |
| Component | Function | (wt. %) | (mg/tab) | (mg/tab) |
| Compound 1A API | Drug Carrier | 25.00 | 150.00 | 250.00 |
| (100% Assay) | | | | |
| AVICEL ® PH-102 | Filler/Diluent | 29.25 | 175.50 | 292.50 |
| (Microcrystalline | | | | |
| Cellulose) | | | | |
| PARTECK ® M100 | Filler/Diluent | 29.25 | 175.50 | 292.50 |
| (Mannitol) | | | | |
| AC-DI-SOL ® | Disintegrant | 3.00 | 18.00 | 30.00 |
| (Croscarmellose | | | | |
| Sodium) | | | | |
| CAB-O-SIL ® | Glidant | 0.50 | 3.00 | 5.00 |
| (Fumed Silica) | | | | |
| Sodium Stearyl | Lubricant | 1.00 | 6.00 | 10.00 |
| Fumarate (PRUV ®) | | | | |
| Intra-Granular | | 88.00 | 528.00 | 880.00 |
| Total: | | | | |
| AVICEL ® PH-200 | Comp Aid | 7.00 | 42.00 | 70.00 |
| (Microcrystalline | | | | |
| Cellulose) | | | | |
| AC-DI-SOL ® | Disintegrant | 3.00 | 18.00 | 30.00 |
| (Croscarmellose | | | | |
| Sodium) | | | | |
| Sodium Stearyl | Lubricant | 2.00 | 12.00 | 20.00 |
| Fumarate (PRUV ®) | | | | |
| Extra-Granular | | 12.00 | 72.00 | 120.00 |
| Total: | | | | |
| Total: | | 100 | 600 | 1000 |
| Target Dose | | | 150 | 250 |
| (mg Compound 1A) | | | | |
| Tablet Shape | | | Oval [bisect] | Oval |

TABLE C
Low drug load tablet formulations

| | | Low Drug Load Formulation | | |
|---|---|---|---|---|
| | | Unit | 150 mg | 250 mg |
| | | Composition | Compound 1A | Compound 1A |
| Component | Function | (wt. %) | (mg/tab) | (mg/tab) |
| Compound 1A API | Drug Carrier | 25.67 | 154.00 | 269.39 |
| (92.8% Assay) | | | | |
| AVICEL ® PH-102 | Filler/Diluent | 28.92 | 173.47 | 283.20 |
| (Microcrystalline | | | | |
| Cellulose) | | | | |
| PARTECK ® M100 | Filler/Diluent | 28.92 | 173.47 | 282.50 |
| (Mannitol) | | | | |
| AC-DI-SOL ® | Disintegrant | 3.00 | 18.00 | 30.00 |
| (Croscarmellose | | | | |
| Sodium) | | | | |
| CAB-O-SIL ® | Glidant | 0.50 | 3.00 | 5.00 |
| (Fumed Silica) | | | | |
| Sodium Stearyl | Lubricant | 1.00 | 6.00 | 10.00 |
| Fumarate (PRUV ®) | | | | |
| Intra-Granular Total: | | 88.00 | 527.95 | 880.00 |
| AVICEL ® PH-200 | Comp Aid | 7.00 | 42.00 | 70.00 |
| (Microcrystalline | | | | |
| Cellulose) | | | | |
| AC-DI-SOL ® | Disintegrant | 3.00 | 18.00 | 30.00 |
| (Croscarmellose | | | | |
| Sodium) | | | | |
| Sodium Stearyl | Lubricant | 2.00 | 12.00 | 20.00 |
| Fumarate (PRUV ®) | | | | |

| | | | |
|---|---|---|---|
| Extra-Granular Total: | 12.00 | 72.00 | 120.00 |
| Total: | 100.00 | 600.00 | 1000.00 |

| Target Dose (mg Compound 1A) | 150 | 250 |
|---|---|---|
| Tablet Shape | Oval | Oval |
| | (bisect) | |

TABLE D
High drug load tablet formulations

| | | High Drug Load Formulation | | | | |
|---|---|---|---|---|---|---|
| | | Unit | 250 mg | 500 mg | 750 mg | 1000 mg |
| | | Composition | Compound 1A | Compound 1A | Compound 1A | Compound 1A |
| Component | Function | (wt. %) | (mg/tab) | (mg/tab) | (mg/tab) | (mg/tab) |
| Compound 1A API | Drug Carrier | 64.96 | 250.00 | 500.00 | 750.00 | 999.99 |
| (100% Assay) | | | | | | |
| AVICEL ® PH-102 | Filler/Diluent | 8.52 | 32.79 | 65.58 | 98.37 | 131.16 |
| (Microcrystalline | | | | | | |
| Cellulose) | | | | | | |
| PARTECK M100 | Filler/Diluent | 8.52 | 32.79 | 65.58 | 98.37 | 131.16 |
| (Mannitol) | | | | | | |
| AC-DI-SOL ® | Disintegrant | 3.00 | 11.55 | 23.09 | 34.64 | 46.18 |
| Croscarmellose | | | | | | |
| Sodium) | | | | | | |
| CAB-O-SIL ® | Glidant | 1.00 | 3.85 | 7.70 | 11.55 | 15.39 |
| (Fumed Silica) | | | | | | |
| Sodium Stearyl | Lubricant | 1.00 | 3.85 | 7.70 | 11.55 | 15.39 |
| Fumarate (PRUV ®) | | | | | | |
| Intra-Granular | | 87.00 | 334.82 | 669.64 | 1004.47 | 1339.28 |
| Total: | | | | | | |
| AVICEL ® PH-200 | Comp Aid | 8.00 | 30.79 | 61.58 | 92.36 | 123.15 |
| (Microcrystalline | | | | | | |
| Cellulose) | | | | | | |
| AC-DI-SOL ® | Disintegrant | 3.00 | 11.55 | 23.09 | 34.64 | 46.18 |
| (Croscarmellose | | | | | | |
| Sodium) | | | | | | |
| Sodium Stearyl | Lubricant | 2.00 | 7.70 | 15.39 | 23.09 | 30.79 |
| Fumarate (PRUV ®) | | | | | | |
| Extra-Granular | | 13.00 | 50.03 | 100.06 | 150.09 | 200.12 |
| Total: | | | | | | |
| Total: | | 100 | 384.85 | 769.7 | 1154.56 | 1539.4 |
| Target Dose | | | 250 | 500 | 750 | 1000 |
| (mg Compound 1A) | | | | | | |
| Tablet Shape | | | Round | Oval | Oval | Oval |

TABLE E
High drug load tablet formulations

| | | High Drug Load Formulation | | | | |
|---|---|---|---|---|---|---|
| | | Unit | 250 mg | 500 mg | 750 mg | 1000 mg |
| | | Composition | Compound 1A | Compound 1A | Compound 1A | Compound 1A |
| Component | Function | (wt. %) | (mg/tab) | (mg/tab) | (mg/tab) | (mg/tab) |
| Compound 1A API (92.8% | Drug Carrier | 70.00 | 269.40 | 538.79 | 808.19 | 1077.58 |
| Assay) | | | | | | |
| AVICEL ® PH-102 | Filler/Diluent | 6.00 | 23.09 | 46.18 | 69.27 | 92.37 |
| (Microcrystalline Cellulose) | | | | | | |
| PARTECK M100 | Filler/Diluent | 6.00 | 23.09 | 46.18 | 69.27 | 92.37 |
| (Mannitol) | | | | | | |
| AC-DI-SOL ® | Disintegrant | 3.00 | 11.55 | 23.09 | 34.64 | 46.18 |
| (Croscarmellose Sodium) | | | | | | |
| CAB-O-SIL ® (Fumed | Glidant | 1.00 | 3.85 | 7.70 | 11.55 | 15.39 |
| Silica) | | | | | | |
| Sodium Stearyl Fumarate | Lubricant | 1.00 | 3.85 | 7.70 | 11.55 | 15.39 |
| (PRUV ®) | | | | | | |
| Intra-Granular Total: | | 87.00 | 334.82 | 669.64 | 1004.46 | 1339.28 |
| AVICEL ® PH-200 | Comp Aid | 8.00 | 30.79 | 61.58 | 92.36 | 123.15 |
| (Microcrystalline Cellulose) | | | | | | |
| AC-DI-SOL ® | Disintegrant | 3.00 | 11.55 | 23.09 | 34.64 | 46.18 |
| (Croscarmellose Sodium) | | | | | | |
| Sodium Stearyl Fumarate | Lubricant | 2.00 | 7.70 | 15.39 | 23.09 | 30.79 |
| (PRUV ®) | | | | | | |

| | | | | | |
|---|---|---|---|---|---|
| Extra-Granular Total: | 13.00 | 50.03 | 100.06 | 150.09 | 200.12 |
| Total: | 100.00 | 384.85 | 769.70 | 1154.56 | 1539.40 |
| Target Dose (mg Compound 1A) | | 250 | 500 | 750 | 1000 |
| Tablet Shape | | Round | Oval | Oval | Oval |

### 5. Methods of Preparation

Any conventional method for obtaining a tablet can be used, for example, the methods described in pharmacopoeias such as the U.S. Pharmacopeia, and the European Pharmacopoeia, may be used.

In certain embodiments, the method for making a tablet comprises the steps of mixing, compacting, roller compacting, milling, final blending, and compressing.

In certain embodiments, the method for making a tablet comprises an intragranular phase. In one embodiment, the intragranular phase comprises blending of API with the intragranular excipients to obtain a raw blend. In one embodiment, the intragranular phase further comprises roller compaction to form ribbons, and milling to produce a controlled granule particle size distribution with improved bulk density and flowability relative to the raw blend.

In certain embodiments, the method for making a tablet comprises an extragranular phase. In one embodiment, the extragranular phase comprises addition of extragranular excipients, including disintegrant(s), glidant(s), and lubricant. In one embodiment, the extragranular excipients are chosen and sized to further optimize flow into the tablet press and to ensure rapid disintegration of granules upon contact with gastric media.

In certain embodiments, the particle size of the excipients is selected such that the blend has good flow that avoids variability in die fill.

In certain embodiments, the particle size of the excipients is selected such that the blend has a reduced risk of content non-uniformity (e.g., out-of-spec blend or tablet CU). In certain embodiments, the particle size of the excipients is selected such that the blend has reduced impurity levels potentially related to non-optimal compaction profiles or non-uniform stress distribution.

In certain embodiments, the particle size distribution was assessed by analytical methodologies, including laser diffraction in appropriate dispersant and sieve analysis for granules. In certain embodiments, advanced characterization (e.g., Raman chemical imaging) techniques were used to assess in tablet API particle size and spatial distribution.

In certain embodiments, the method provided herein is for making a coated tablet of about 100 to about 1500 mg of Compound 1. In certain embodiments, the method provided herein is for making a tablet of about 100 mg, about 150 mg, about 200 mg, about 250 mg, about 500 mg, about 750 mg or about 1000 mg of Compound 1.

In certain embodiments, the method provided herein is for making a tablet of about 100 to about 1000 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In certain embodiments, the method provided herein is for making a tablet of about 100 mg, about 150 mg, about 200 mg, about 250 mg, about 500 mg, about 750 mg or about 1000 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In certain embodiments, the method for making a tablet comprises the steps of mixing, compacting, roller compacting, milling, final blending, and compressing. In certain embodiments, the method provided herein is for making a tablet of about 100 to about 500 mg of Compound 1. In certain embodiments, the method provided herein is for making a tablet of about 100 mg, about 150 mg, about 200 mg, about 250 mg or about 300 mg of Compound 1.

In certain embodiments, the method provided herein is for making a tablet of about 100 to about 300 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In certain embodiments, the method provided herein is for making a tablet of about 100 mg, about 150 mg, about 200 mg, about 250 mg or about 300 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In certain embodiments, the method provided herein is for making a tablet of about 100 to about 300 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium. In certain embodiments, the method provided herein is for making a tablet of about 154 mg or about 269.40 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium.

In certain embodiments, the method provided herein is for making a tablet of about 100 to about 1000 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium. In certain embodiments, the method provided herein is for making a tablet of about 269.40 mg, about 538.79 mg or about 808.19 mg of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium.

In one embodiment, the method for making a tablet comprises blending Compound 1 with an intragranular excipient and an extragranular excipient and compressing with a compression tooling.

In certain embodiments, the method for making a tablet comprises one or more of the following steps: 1) blending, 2) compaction simulating, 3) roller compacting, 4) milling, 5) final blending, and 6) compression.

In one embodiment, the blending step comprises mixing Compound 1 with intragranular components including, a diluent, a disintegrant, a glidant and a lubricant to obtain a blend.

In one embodiment, the blend is compacted by a compaction simulator followed by a roller compactor to obtain a ribbon, which is milled.

In one embodiment, the milled granules are mixed with extra-granular excipients including a compression aid, a disintegrant, a glidant and a lubricant to obtain a final blend. The final blend is then compressed to obtain the tablet.

Examples of the equipment used in the processes for making a tablet provided here include, roller compacters, blenders, smooth rollers, tabletop/floor balances, tablet presses, tooling such as standard, round tooling, oval tooling, calipers, disintegration apparatus, friability testers and hardness testers.

Any tableting conditions suitable for tablet molding can be used. In certain embodiments, tableting force is used such that the tablets are not damaged during the manufacturing process.

Any tablet hardness suitable for tablet molding can be used.

Any tablet thickness suitable for tablet molding can be used.

The tablet provided herein has a good stability during storage. In one embodiment, dissolution of the tablet is not reduced for up to at least 1 month, at least 2 months, at least 3 months, at least 4 months, at least 6 months, at least 8 months, at least 10 months, or at least 12 months, in a conventional packaging.

### 6. Methods of Use

The pharmaceutical compositions provided herein are useful for ameliorating, treating, and/or preventing a variety of diseases, conditions, and/or disorders. In particular embodiments, the pharmaceutical compositions may be useful for treating conditions in which inhibition of an interleukin-1 receptor-associated kinase (IRAK) pathway is therapeutically useful. In some embodiments, the solid forms of Compound 1 directly inhibit an IRAK protein, such as IRAK1, IRAK2, IRAK3 and/or IRAK4. In certain embodiments, the pharmaceutical compositions are useful for treating, preventing, and/or ameliorating auto-immune diseases, inflammatory disorders, cardiovascular diseases, nerve disorders, neurodegenerative disorders, allergic disorders, asthma, pancreatitis, multi-organ failure, kidney diseases, platelet aggregation, cancer, transplantation, sperm motility, erythrocyte deficiency, graft rejection, lung injuries, respiratory diseases, ischemic conditions, and bacterial and viral infections.

In some embodiments, the pharmaceutical compositions may be used to treat or prevent allergic diseases, amyotrophic lateral sclerosis (ALS), systemic lupus erythematosus, rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmyopathy, or asthma.

The pharmaceutical compositions provided herein may also be useful for ameliorating, treating, and/or preventing immune regulatory disorders related to bone marrow or organ transplant rejection or graft-versus-host disease. Examples of inflammatory and immune regulatory disorders that can be treated with the solid forms of Compound 1 include, but are not limited to, transplantation of organs or tissue, graft-versus-host diseases brought about by transplantation, autoimmune syndromes including rheumatoid arthritis, lupus, including systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, erythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA bullous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C4 release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic liver disease, including alcoholic cirrhosis, non-alcoholic steatohepatitis (NASH), hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, Parkinson's disease, trauma, chronic bacterial infection, palmoplantar pustulosis, hidradenitis suppurativa, cytokine release syndrome (CRS), acute respiratory distress syndrome (ARDS), acute kidney injury (AKI), kidney malfunction, or thrombosis.

In some embodiments, the disease or condition is a lymphoid neoplasm, a myeloid neoplasm or a myeloid/lymphoid neoplasm. In some embodiments, the disease or condition is hidradenitis suppurativa, or a lymphoid, myeloid or myeloid/lymphoid neoplasm selected from myeloproliferative neoplasms (MPN), myeloid/lymphoid neoplasms with PDGFRA rearrangement, myeloid/lymphoid neoplasms with PDGFRB rearrangement, myeloid/lymphoid neoplasms with FGFR1 rearrangement, myeloid/lymphoid neoplasms with PCM1-JAK2, myelodysplastic/myeloproliferative neoplasms (MDS/MPN), myeloid sarcoma, myeloid proliferations related to Down syndrome, blastic plasmacytoid dendritic cell neoplasm, B-lymphoblastic leukemia/lymphoma; and/or T-lymphoblastic leukemia/lymphoma. In some embodiments, the myeloid neoplasm is a myeloproliferative neoplasm selected from chronic myeloid leukemia (CML), chronic neutrophilic leukemia (CNL), primary myelofibrosis (PMF), essential thrombocythemia, chronic eosinophilic leukemia, or a combination thereof. In other embodiments, the myeloid neoplasm is a myelodysplastic/myeloproliferative neoplasm. In other embodiments, the myelodysplastic/myeloproliferative neoplasm is selected from chronic myelomonocytic leukemia, atypical chronic myeloid leukemia (aCML), juvenile myelomonocytic leukemia (JMML), MDS/MPN with ring sideroblasts and thrombocytosis (MDS/MPN-RS-T), or a combination thereof.

In certain embodiments, the pharmaceutical compositions are useful for treating nerve pain, including neuropathic pain and inflammation induced pain.

In certain embodiments, the pharmaceutical compositions are useful for treating and/or preventing arthralgia, arthritis, rheumatoid arthritis, psoriatic arthritis, osteoarthritis, systemic lupus erythematosus, lupus nephritis, ankylosing spondylitis, osteoporosis, systemic sclerosis, multiple sclerosis, psoriasis, in particular pustular psoriasis, type I diabetes, type II diabetes, inflammatory bowel disease (Crohn's disease and ulcerative colitis), hyperimmunoglobulinemia d and periodic fever syndrome, cryopyrin-associated periodic syndromes, Schnitzler's syndrome, systemic juvenile idiopathic arthritis, adult's onset Still's disease, gout, gout flares, pseudogout, SAPHO (Synovitis, Acne, Pustulosis, Hyperostosis, and Osteitis) syndrome, Castleman's disease, sepsis, stroke, atherosclerosis, celiac disease, DIRA (deficiency of Il-1 receptor antagonist), Alzheimer's disease, or Parkinson's disease.

Proliferative diseases that may be treated by the pharmaceutical compositions herein include benign or malignant tumors, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, IL-1 driven disorders, a MyD88 driven disorder (such as ABC diffuse large B-cell lymphoma (DLBCL), Waldenstrom's macroglobulinemia, Hodgkin's lymphoma, primary cutaneous T-cell lymphoma or chronic lymphocytic leukemia), smoldering or indolent multiple myeloma, or hematological malignancies (including leukemia, acute myeloid leukemia (AML), DLBCL, ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndrome (MDS), clonal cytopenia of undetermined significance (CCUS), myelofibrosis, polycythemia vera, Kaposi's sarcoma, Waldenstrom's macroglobulinemia (WM), splenic marginal zone lymphoma, multiple myeloma, plasmacytoma, intravascular large B-cell lymphoma). In particular, the solid forms of Compound 1 are useful in treating drug resistant malignancies, such as those resistant to JAK inhibitors ibrutinib resistant malignancies, including ibrutinib resistant hematological malignancies, such as ibrutinib resistant CLL and ibrutinib resistant Waldenstrom's macroglobulinemia.

In one embodiment, myelodysplastic syndrome (MDS) is relapsed, resistant or refractory MDS. In one embodiment, MDS is refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms or chronic myelomonocytic leukemia (CMML). In some embodiments, the MDS is very low risk, low risk, intermediate risk, high risk or very high risk MDS based on the Revised International Prognostic Scoring System (IPSS-R). In some embodiments, the MDS is very low, low, or intermediate-1 risk (IPSS-R≤3.5), collectively, lower-risk MDS (LR-MDS). In some embodiments, the MDS patient has bone marrow blast ≤5%. In one embodiment, the MDS is very low risk. In another embodiment, the MDS is low risk. In another embodiment, the MDS is intermediate risk. In another embodiment, the MDS is high risk. In another embodiment, the MDS is very high risk MDS. In some embodiments, the MDS is IPSS-R intermediate, high or very high risk MDS (IPSS-R>3.5), collectively, higher-risk MDS (HR-MDS). In some embodiments, the MDS is primary or de novo MDS. In other embodiments, the MDS is secondary MDS.

In yet certain embodiments, the MDS is transfusion dependent (TD) lower risk MDS (LR-MDS). In yet certain embodiments, the MDS is high transfusion burden MDS, wherein the patient requires about or greater than 8 red blood cell units for over 8 weeks or for over 16 weeks. In yet certain embodiments, the MDS is a low transfusion burden MDS, wherein the patient requires from about 3 to about 7 red blood cell units for over 8 weeks or for over 16 weeks. In yet certain embodiments, the MDS is transfusion dependent MDS wherein the patient requires about 4 or more red blood cell units over 8 weeks who have not responded to or have lost response to or are ineligible for erythropoiesis-stimulating agents (ESAs). In yet certain embodiments, the MDS is transfusion dependent MDS wherein the patient requires about 2 or more red blood cell units over 8 weeks. In yet certain embodiments, the MDS is MDS with symptomatic anemia with hemoglobin ≤9.0 g/dL and no red blood cell (RBC) transfusion for at least 16 weeks prior to administration of Compound 1. In yet certain embodiments, the MDS is transfusion dependent MDS, wherein the patient requires about or greater than 2 red blood cell units within 8 weeks in the preceding 16 weeks for a hemoglobin <9.0 g/dL prior to administration of Compound 1. In yet certain embodiments, the MDS is relapsed, refractory or ineligible for ESAs and have previously received one or more prior approved therapies for LR-MDS. In yet certain embodiments, the MDS is MDS with del (5q) mutation and which has failed prior lenalidomide therapy. In yet certain embodiments, the MDS is LR-MDS wherein the patient has transfusion-dependent anemia. In certain embodiments, the MDS is transfusion-dependent MDS which is relapsed, refractory to or have had inadequate response to prior therapies for MDS or to HMAs. In yet certain embodiments, the MDS is ring sideroblast-negative (RS-negative MDS). In yet certain embodiments, the MDS is ring sideroblast-negative MDS wherein the patient is relapsed, refractor or ineligible for ESAs. In yet certain embodiments, the MDS is very low risk, low risk, moderate low risk, moderate high risk, high risk or very high risk MDS based on the Molecular International Prognostic scoring system IPSS-M. In one embodiment, MDS has a prognostic score of <=3 IPSS. In one embodiment, the MDS is relapsed, refractory to prior therapy such as luspatercept or imetelstat. In yet certain embodiments, the MDS is relapsed, refractory and/or eligible for ESAs. In yet certain embodiments, the MDS is HMA naïve. In one embodiment, the MDS is previously-treated transfusion dependent LR-MDS.

In one embodiment:
(i) the myelodysplastic syndrome is a higher-risk myelodysplastic or a lower-risk myelodysplastic syndrome;
(ii) the myelodysplastic syndrome is a refractory or relapsed lower-risk myelodysplastic syndrome;
(iii) the myelodysplastic syndrome is a relapsed, refractory/resistant, intolerant, or has an inadequate response to one or more therapies for myelodysplastic syndrome;
(iv) the myelodysplastic syndrome is a lower-risk myelodysplastic syndrome that is relapsed, refractory/resistant, intolerant, or has an inadequate response to one or more therapies for lower-risk myelodysplastic syndrome;
(v) the myelodysplastic syndrome is a transfusion-dependent lower-risk myelodysplastic syndrome;
(vi) the myelodysplastic syndrome is a transfusion-dependent lower-risk myelodysplastic syndrome that is non-responsive to or ineligible for treatment with erythropoiesis-stimulating agents;
(vii) the myelodysplastic syndrome is a lower-risk myelodysplastic syndrome that is relapsed, refractory to or ineligible for treatment with erythropoiesis-stimulating agents;
(viii) the myelodysplastic syndrome is a lower-risk myelodysplastic syndrome that is relapsed, refractory to or ineligible for treatment with erythropoiesis-stimulating agents and has previously been treated with one or more therapies for lower-risk myelodysplastic syndrome; or
(ix) the myelodysplastic syndrome is a transfusion-dependent lower-risk myelodysplastic syndrome that is relapsed, refractory to or ineligible for treatment with erythropoiesis-stimulating agents and has been previously received with one or more therapies for lower-risk myelodysplastic syndrome.

Examples of allergic disorders that may be treated using the compositions provided herein, include, but are not limited to, asthma (e.g. atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma), allergic bronchopulmonary aspergillosis (ABPA), allergic rhinitis, perennial allergic rhinitis, perennial rhinitis, vasomotor rhinitis, post-nasal drip, purulent or non-purulent sinusitis, acute or chronic sinusitis, and ethmoid, frontal, maxillary, or sphenoid sinusitis.

As another example, rheumatoid arthritis (RA) typically results in swelling, pain, loss of motion and tenderness of target joints throughout the body. RA is characterized by chronically inflamed synovium that is densely crowded with lymphocytes. The synovial membrane, which is typically one cell layer thick, becomes intensely cellular and assumes a form similar to lymphoid tissue, including dendritic cells, T-, B- and NK cells, macrophages and clusters of plasma cells. This process, as well as a plethora of immunopathological mechanisms including the formation of antigen-immunoglobulin complexes, eventually result in destruction of the integrity of the joint, resulting in deformity, permanent loss of function and/or bone erosion at or near the joint. The disclosed solid forms of Compound 1, or compositions thereof, may be used to treat, ameliorate, or prevent any single, several or all of these symptoms of RA. Thus, in the context of RA, the solid forms of Compound 1 are considered to provide therapeutic benefit when a reduction or amelioration of any of the symptoms commonly associated with RA is achieved, regardless of whether the treatment results in a concomitant treatment of the underlying RA and/or a reduction in the amount of circulating rheumatoid factor (“RF”).

The American College of Rheumatology (ACR) has developed criteria for defining improvement and clinical remission in RA. Once such parameter, the ACR20 (ACR criteria for 20% clinical improvement), requires a 20% improvement in the tender and swollen joint count, as well as a 20% improvement in 3 of the following 5 parameters: patient's global assessment, physician's global assessment, patient's assessment of pain, degree of disability, and level of acute phase reactant. These criteria have been expanded for 50% and 70% improvement in ACR50 and ACR70, respectively. Other criteria include Paulu's criteria and radiographic progression (e.g. Sharp score).

In some embodiments, therapeutic benefit in patients suffering from RA is achieved when the patient exhibits an ACR20. In specific embodiments, ACR improvements of ACRC50 or even ACR70 may be achieved.

Cytokine release syndrome (CRS) is a potentially life-threatening condition that may result from a variety of factors, including severe viral infections such as influenza, administration of antibodies that are used for immunotherapy, such as cancer immunotherapy, and non-protein-based cancer drugs such as oxaliplatin and lenalidomide. Immunotherapy can involve high levels of immune activation that exceed naturally occurring immune activation levels, and CRS is a non-antigen specific toxicity that can occur as a result. As immune-based therapies become more potent, CRS is becoming increasing diagnosed. CRS has also been observed in the setting of haploidentical donor stem cell transplantation, and graft-versus-host disease. Shimabukuro-Vornhagen et al., Journal for ImmunoTherapy of Cancer 6:56 (2018). CRS is associated with elevated circulating levels of several cytokines including interleukin (IL)-6 and interferon 7. Lee et al., Blood 124(2):188-195 (10 Jul. 2014; Epub 29 May 2014).

CRS typically is clinically observed when significant numbers of lymphocytes and/or myeloid cells are activated and release inflammatory cytokines. The cytokine release may be induced by chemo- or biotherapy, and/or may be associated with therapeutic antibody treatments, such as immunotherapy, for example, for cancer treatment. Exemplary immunotherapies that may result in CRS include, but are not limited to, therapies where the cells express recombinant receptors, such as chimeric antigen receptors (CARs) and/or other transgenic receptors such as T cell receptors (TCRs). CRS induced by CAR T therapy generally occurs within days of T cell infusion at the peak of CAR T cell expansion. Giavridis et al., Nat Med. 24(6):731-738 (June 2018; Epub 28 May 2018). Examples of CAR T therapy that can induce CRS include axicabtagene ciloleucel (marketed as YESCARTA®) and tisagenlecleucel (marketed as KYMRIAH®).

Highly elevated interleukin 6 (IL-6) levels have been observed in patients with CRS and also in murine models of the disease, indicating that IL-6 may have a role in CRS pathophysiology. Shimabukuro-Vornhagen, J Immunother Cancer 6(1), 56 (2018). IL-6 can signal via two different modes. Classical IL-6 signaling involves binding of IL-6 to a membrane-bound IL-6 receptor. However, the IL-6 receptor does not possess intracellular signaling domains. Instead, after soluble IL-6 binds to membrane-bound IL-6 receptors, the IL-6/IL-6 receptor complex binds to membrane-bound gp130, which initiates signaling through its intracellular domain. In trans-signaling, IL-6 binds to a soluble form of the IL-6 receptor, which is typically cleaved from the cell surface by metalloproteinases. The resulting soluble IL-6/IL-6 receptor complex binds to gp130 and therefore can also induce signaling in cell types that do not express membrane bound IL-6 receptors.

IL-6 contributes to many of the key symptoms of CRS. Via trans-signaling, IL-6 leads to characteristic symptoms of severe CRS, i.e. vascular leakage, and activation of the complement and coagulation cascade inducing disseminated intravascular coagulation (DIC). In addition, IL-6 likely contributes to cardiomyopathy that is often observed in patients with CRS by promoting myocardial dysfunction. In a murine model, CRS developed within 2-3 days of CAR T cell infusion and could be lethal. Giavridis et al., Nat Med. 24(6): 731-738 (2018). CRS symptoms may start within minutes or hours of the start of antibody treatment, and can include a fever, which may reach or exceed 40° C., nausea, fatigue, headache, tachycardia, hypotension, rash, shortness of breath, and/or myalgias. However, in certain cases, additional and potentially more serious complications may develop, including cardiac dysfunction, adult respiratory distress syndrome, neurological toxicity, renal and/or hepatic failure, and/or disseminated intravascular coagulation.

The National Cancer Institute Common Terminology Criteria for Adverse Events (CTCAE v. 5.0, pub. Nov. 27, 2017) includes a grading system for CRS.
Grade 1: Fever with or without constitutional symptoms.
Grade 2: Hypotension responding to fluids; hypoxia responding to <40% 02.
Grade 3: Hypotension managed with one pressor; hypoxia requiring ≥40% 02.
Grade 4: Life-threatening consequences; urgent intervention indicated.
Grade 5: Death.

The subject may not exhibit a sign or symptom of CRS and/or may be at risk of developing CRS. In such embodiments, administering the solid forms of Compound 1 substantially prevents the onset of CRS, or prevents the onset of grade 2 or higher CRS.

Alternatively, the subject exhibits at least one sign or symptom of CRS and may exhibit at least one sign or symptom of grade 1 CRS. Or the subject may exhibit at least one sign or symptom of grade 2 or higher CRS, such as grade 3 or higher CRS. The disclosed solid forms of Compound 1 may be administered within 24 hours of the onset of the sign or symptom, and/or administering the solid forms of Compound 1 may ameliorate the sign or symptom of CRS, compared to the severity of the sign or symptom prior to administration of the solid forms of Compound 1, such as reducing the grade of CRS from 4 to 3, 2 or 1, or from 3, to 2 or 1, or from 2 to 1. Alternatively, CRS symptoms are substantially reduced to below grade 1 level, such that the subject no longer experiences symptoms associated with CRS. In some embodiments the sign or symptom is a fever and may be a fever of 40° C. or higher.

The pharmaceutical compositions may be administered to a subject that has previously been administered a first therapy for which CRS is a known, suspected, or potential side effect. Administration of the first therapy may be initiated from greater than zero to 10 days, or longer, prior to administration of the solid forms of Compound 1. Alternatively, the solid forms of Compound 1 may be administered to a subject who will be, or is concurrently being, administered a first therapy for which CRS is a known, suspected, and/or potential side effect. The first therapy may comprise a cell therapy, including, but not limited to, chimeric antigen receptor (CAR)-expressing therapy and/or a transgenic receptor therapy. Cell-free antibodies are also known to elicit this syndrome, particularly those that activate T-cells.

A second therapeutic agent, for example, a steroid, an anti-inflammatory agent, an immunosuppressant, or a combination thereof, also may be administered to treat or prevent CRS. The disclosed solid forms of Compound 1 may be administered substantially simultaneously with the second therapeutic agent, or the solid forms of Compound 1 and second therapeutic agent may be administered sequentially in any order.

Acute respiratory distress syndrome (ARDS) is a syndrome characterized by a severe shortness of breath, labored and unusually rapid breathing, low blood pressure, confusion and extreme tiredness. This syndrome can be diagnosed based on a PaO₂/FiO₂ ratio of less than 300 mmHg despite a PEEP of more than 5 cm H₂O (Fan et al JAMA. 319: 698-71).

ARDS occurs when fluid builds up in lung alveoli. The fluid prevents the lungs from filling with enough air, limiting the amount of oxygen that reaches the bloodstream which, in turn, deprives the organs of the oxygen they need to function. The symptoms of ARDS can vary in intensity, depending on its cause and severity. Severe shortness of breath—the hallmark of ARDS—usually develops within a few hours to a few days after the infection by some respiratory viruses, e.g., COVID-19 and influenza. Many people who develop ARDS do not survive, and the risk of death increases with age and severity of illness. Of the patients that survive ARDS, some completely recover while others have lasting damage to their lungs. ARDS may be referred to as Acute Lung Injury (ALI) in some publications.

Acute kidney injury (AKI), also known as acute renal injury (ARI) or acute renal failure (ARF), is a syndrome characterized by an abrupt reduction of renal function including, e.g., the ability to excrete waste from a patient's blood. AKI is characterized by a decline of glomerular filtration rate, urine output, or both. This loss of filtration capacity results in retention of nitrogenous (urea and creatinine) and non-nitrogenous waste products that are normally excreted by the kidney, a reduction in urine output, or both. AKI may be categorized as prerenal, intrinsic renal, or postrenal in causation. Intrinsic renal disease can be further divided into glomerular, tubular, interstitial, and vascular abnormalities. AKI is accompanied by an inflammatory response that if unchecked can lead to renal fibrosis and chronic renal failure. AKI usually occurs over a period of hours or days and is potentially reversible. AKI may be characterized as an abrupt (i.e., for example, within 14 days, within 7 days, within 72 hours, or within 48 hours) reduction in kidney function identified by an absolute increase in serum creatinine of greater than or equal to 0.3 mg/dl (≥26.4 mol/l), a percentage increase in serum creatinine of greater than or equal to 50% (1.5-fold from baseline), or a reduction in urine output (documented oliguria of less than 0.5 ml/kg per hour for at least 6 hours). Risk factors include, for example, a subject undergoing or having undergone major vascular surgery, coronary artery bypass, or other cardiac surgery; a subject having pre-existing congestive heart failure, preeclampsia, eclampsia, diabetes mellitus, hypertension, coronary artery disease, proteinuria, renal insufficiency, glomerular filtration below the normal range, cirrhosis, serum creatinine above the normal range, or sepsis; or a subject exposed to NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin. This list is not meant to be limiting.

Kidney malfunction includes, but is not limited to, kidney disorders, kidney disease, kidney dysfunction, kidney cancer, absence of at least one kidney due to accidents, surgical removal or genetic disorders, or other conditions where one or both of the kidneys are not properly functioning. Kidney malfunction may include acute kidney injury.

Thrombosis is a clotting disorder to which an excess of platelets contributes. Thrombosis may refer to the formation of a thrombus (blood clot) inside a blood vessel. The term encompasses, without limitation, arterial and venous thrombosis, including deep vein thrombosis, portal vein thrombosis, jugular vein thrombosis, renal vein thrombosis, stroke, myocardial infarction, Budd-Chiari syndrome, Paget-Schroetter disease, and cerebral venous sinus thrombosis. In some embodiments, the patient is at heightened risk relative to the general population (e.g., as measured by recognized risk factors) of a thrombotic event. In some embodiments, a patient has one or more risk factors that make the patient have a high risk of developing thrombosis relative to the general population. Risk factors for thrombosis include, e.g., classical cardiovascular disease risk factors: hyperlipidemia, smoking, diabetes, hypertension, and abdominal obesity; strong classical venous thromboembolism risk factors: trauma or fractures, major orthopedic surgery, and oncological surgery; moderate classical venous thromboembolism risk factors: non-oncological surgery, oral contraceptives and hormone replacement therapy, pregnancy and puerperium, hypercoagulability, and previous venous thromboembolism; and weak classical venous thromboembolism risk factors: age, bed rest (>3 days), prolonged travel, and metabolic syndrome. Additional risk factors include inherited, acquired and mixed coagulation or metabolic risk factors for thrombosis such as, e.g., inherited: antithrombin deficiency, protein C deficiency, Protein S deficiency, Factor V Leiden, Prothrombin G20210A; acquired: antiphospholipid syndrome; mixed: hyperhomocysteinaemia, increased fibrinogen levels, increased factor VIII levels, increased factor IX levels. In some cases, the use of heparin may increase the risk of thrombosis including, e.g., heparin-induced thrombocytopenia (HIT). Diseases and conditions associated with thrombosis include, without limitation, acute venous thrombosis, pulmonary embolism, thrombosis during pregnancy, hemorrhagic skin necrosis, acute or chronic disseminated intravascular coagulation (DIC), sepsis induced coagulopathy (SIC), clot formation from surgery, long bed rest, long periods of immobilization, venous thrombosis, fulminant meningococcemia, acute thrombotic stroke, acute coronary occlusion, acute peripheral arterial occlusion, massive pulmonary embolism, axillary vein thrombosis, massive iliofemoral vein thrombosis, occluded arterial cannulae, occluded venous cannulae, cardiomyopathy, venoocclusive disease of the liver, hypotension, decreased cardiac output, decreased vascular resistance, pulmonary hypertension, diminished lung compliance, leukopenia, thrombocytopenia (e.g., immune thrombocytopenia), and immune thrombocytic purpura. In a subject at risk for thrombosis, the subject may be monitored using methods known to those of skill in the art of maintaining hemostasis in patients at risk for thrombosis. Examples of methods for monitoring patients at risk of thrombosis included, without limitation, digital subtraction angiography, in vitro assays or non-invasive methods. Examples of in vitro assays useful for identifying and monitoring subjects at risk for thrombosis and for treatment using the present methods include, without limitation, functional assays and antibody detection assays.

Thrombotic event refers to any disorder which involves a blockage or partial blockage of an artery or vein with a thrombosis. A thrombotic event includes, but is not limited to, thrombotic disorders such as myocardial infarction, unstable angina, stroke, pulmonary embolism, transient ischemic attack, deep vein thrombosis, thrombotic re-occlusion and peripheral vascular thrombosis. A thrombotic event also includes thrombotic re-occlusion which occurs subsequent to a coronary intervention procedure or thrombolytic therapy.

COVID-19 is a disease caused by infection by SARS-CoV-2 (previously known as 2019-nCoV) which first appeared in Wuhan, China.

COVID-19-associated ARDS refers to ARDS that is caused by infection by SARS-CoV-2. Patients having COVID-19-associated ARDS may have been diagnosed as having a COVID-19, may have been exposed to another person having a COVID19, or may be suspected of having a COVID-19 based on their symptoms.

COVID-19-associated AKI refers to AKI that is caused by infection by SARS-CoV-2. Patients having COVID-19-associated AKI may have been diagnosed as having a COVID-19, may have been exposed to another person having a COVID-19, or may be suspected of having a COVID-19 based on their symptoms. In some cases, COVID-19-associated AKI includes AKI with the symptoms described, e.g., in Batlle et al. J. AM. SOC. NEPHROL. 2020, 31(7): 1380-1383 and Gabarre et al. Intensive Care Med. 2020, 46(7): 1339-1348, the disclosures of which are incorporated herein by reference in their entireties.

COVID-19-associated thrombosis refers to thrombosis that is caused by infection by SARS-CoV-2. Patients having COVID-19-associated thrombosis may have been diagnosed as having a COVID-19, may have been exposed to another person having a COVID-19, or may be suspected of having a COVID-19 based on their symptoms. In some cases, COVID-19-associated thrombosis includes any of the symptoms described in, e.g., Connors et al. Blood 2020, 135(23): 2033-2040 and Bikdeli et al. J. Am. Coll. Cardiol. 2020, 75(23): 2950-73, the disclosures of which are incorporated herein by reference in their entireties.

The term “associated with COVID-19” refers to a symptom or indication that typically develops within 28 days of hospitalization due to/signs of COVID-19.

For COVID-19-associated ARDS, successful treatment may include a decrease in shortness of breath, less labored or less rapid breathing, higher blood pressure, decreased confusion and/or a decrease tiredness. A treatment may be administered prophylactically, i.e., before the onset of ARDS. A prophylactic treatment prevents ARDS and can be administered to patients that have or are suspected of having a COVID-19 infection, but without the severe symptoms of ARDS. For example, prophylactic treatment can be administered to patients that have a cough without the other symptoms of ARDS.

For COVID-19-associated AKI, successful treatment may include increased kidney function. Kidney function may be assessed by measuring serum creatinine levels, serum creatinine clearance, or blood urea nitrogen levels. In some cases, the successful treatment includes a reduction in metabolic acidosis, hyperkalaemia, oliguria or anuria, azotemia, restoration in body fluid balance, and improved effects on other organ systems. A treatment may be administered prophylactically, i.e., before the onset of AKI. A prophylactic treatment prevents AKI and can be administered to patients that have or are suspected of having a COVID-19 infection, but without the severe symptoms of AKI. For example, prophylactic treatment can be administered to patients that have one or more of increased serum or urine creatinine, hematuria, hypoproteinemia, decreased antithrombin III levels, hypalbuminaemia, leucozyturia, or proteinuria without the other symptoms of AKI.

For COVID-19-associated thrombosis, successful treatment may include improvement in the subject's coagulation profile, or preventing, slowing, delaying, or arresting, a worsening of the coagulation profile for which the subject is at risk. A coagulation profile may be assessed by measurement of one or more coagulation parameters including, e.g., a subject's serum level of one or more of D-dimer, Factor II, Factor V (e.g., Factor V Leiden), Factor VII, Factor VIII, Factor IX, Factor XI, Factor XII, Factor XIII, F/fibrin degradation products, thrombin-antithrombin 111 complex, fibrinogen, plasminogen, prothrombin, and von Willebrand factor. Additional coagulation parameters that may be measured for the coagulation profile include, e.g., prothrombin time, thromboplastin time, activated partial thromboplast time (aPTT), antithrombin activity, platelet count, protein C levels, and protein S levels. In addition, the levels of C reactive protein may also be assessed in the patient prior to treatment and if elevated this may be used as a further indicator as to an increased risk of thrombosis in the patient.

Sepsis is a clinical syndrome of life-threatening organ dysfunction caused by a dysregulated immune response to infection. The more severe form of sepsis “septic shock” is characterized by a critical reduction in tissue perfusion; acute failure of multiple organs, including the lungs, kidneys, and liver. Common causes in immunocompetent patients include many different species of gram-positive and gram-negative bacteria. Immunocompromised patients may have uncommon bacterial or fungal species as a cause. Signs include fever, hypotension, oliguria, and confusion. Diagnosis is primarily clinical combined with culture results showing infection; early recognition and treatment is critical. Treatment is aggressive fluid resuscitation, antibiotics, surgical excision of infected or necrotic tissue and drainage of pus, and supportive care.

Influenza is a disease generally known as the “flu.” Influenza is caused by a group of viruses that can be broken down into 4 separate groups: Influenza A, Influenza B, Influenza C and Influenza D which are separated based on their nuceloproteins and matrix proteins. Influenza causes viral respiratory infection resulting in fever, coryza, cough, headache, and malaise. Influenza A, B, and C all infect humans while there have been no documented cases of human Influenza D infection. Influenza C on the other hand does not cause typical influenza illness seen in individuals infected with Influenza A, B or C.

Influenza A strains are further classified based on two surface proteins, hemagglutinin (H) and neuraminidase (N). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in people include: A(H1N1) and A(H3N2).

Cytokine release-related condition associated with influenza refers to any condition associated with influenza that leads to high levels of cytokine releases in the lungs and/or kidneys. Cytokine releases-related conditions, include without limitation, influenza-associated ARDS, influenza-associated AKI, influenza-associated thrombosis, influenza-associated sepsis, influenza-associated septic shock, etc.

Influenza-associated ARDS is ARDS that is caused by influenza infection. Patients having influenza-associated ARDS may have been diagnosed as having an influenza infection, may have been exposed to another person having an influenza infection, or may be suspected of having an influenza infection based on their symptoms.

Influenza-associated AKI is AKI that is caused by influenza infection. Patients having influenza-associated AKI may have been diagnosed as having an influenza infection, may have been exposed to another person having an influenza infection, or may be suspected of having an influenza infection based on their symptoms. In some cases, influenza-associated AKI includes AKI with the symptoms described, e.g., in Batlle et al. J. AM. SOC. NEPHROL. 2020, 31(7): 1380-1383 and Gabarre et al. Intensive Care Med. 2020, 46(7): 1339-1348, the disclosures of which are incorporated herein by reference in their entireties.

Influenza-associated thrombosis is thrombosis that is caused by influenza infection. Patients having influenza-associated thrombosis may have been diagnosed as having an influenza infection, may have been exposed to another person having an influenza infection, or may be suspected of having an influenza infection based on their symptoms. In some cases, influenza-associated thrombosis includes any of the symptoms described in, e.g., Connors et al. Blood 2020, 135(23): 2033-2040 and Bikdeli et al. J. Am. Coll. Cardiol. 2020, 75(23): 2950-73, the disclosures of which are incorporated herein by reference in their entireties.

Influenza-associated sepsis is sepsis that is caused by influenza infection. Patients having influenza-associated sepsis may have been diagnosed as having an influenza infection, may have been exposed to another person having an influenza infection, or may be suspected of having an influenza infection based on their symptoms. In some cases, influenza-associated thrombosis includes any of the symptoms described in, e.g., Florescu et al. Virulence. 2014 Jan. 1; 5(1): 137-142. and Gu et al. Eur Respir Rev. 2020 Jul. 21; 29(157):200038, the disclosures of which are incorporated herein by reference in their entireties.

The term “associated with influenza” refers to a symptom or indication that develops within 28 days of hospitalization/signs of influenza infection.

For influenza-associated ARDS, successful treatment may include a decrease in shortness of breath, less labored or less rapid breathing, higher blood pressure, decreased confusion and/or a decrease tiredness. A treatment may be administered prophylactically, i.e., before the onset of ARDS. A prophylactic treatment prevents ARDS and can be administered to patients that have or are suspected of having an influenza infection, but without the severe symptoms of ARDS. For example, prophylactic treatment can be administered to patients that have a cough without the other symptoms of ARDS.

For influenza-associated AKI, successful treatment may include increased kidney function. Kidney function may be assessed by measuring serum creatinine levels, serum creatinine clearance, or blood urea nitrogen levels. In some cases, the successful treatment includes a reduction in metabolic acidosis, hyperkalaemia, oliguria or anuria, azotemia, restoration in body fluid balance, and improved effects on other organ systems. A treatment may be administered prophylactically, i.e., before the onset of AKI. A prophylactic treatment prevents AKI and can be administered to patients that have or are suspected of having an influenza infection, but without the severe symptoms of AKI. For example, prophylactic treatment can be administered to patients that have one or more of increased serum or urine creatinine, hematuria, hypoproteinemia, decreased antithrombin III levels, hypalbuminaemia, leucozyturia, or proteinuria without the other symptoms of AKI.

For influenza-associated thrombosis, successful treatment may include improvement in the subject's coagulation profile, or preventing, slowing, delaying, or arresting, a worsening of the coagulation profile for which the subject is at risk. A coagulation profile may be assessed by measurement of one or more coagulation parameters including, e.g., a subject's serum level of one or more of D-dimer, Factor II, Factor V (e.g., Factor V Leiden), Factor VII, Factor VIII, Factor IX, Factor XI, Factor XII, Factor XIII, F/fibrin degradation products, thrombin-antithrombin 111 complex, fibrinogen, plasminogen, prothrombin, and von Willebrand factor. Additional coagulation parameters that may be measured for the coagulation profile include, e.g., prothrombin time, thromboplastin time, activated partial thromboplast time (aPTT), antithrombin activity, platelet count, protein C levels, and protein S levels. In addition, the levels of C reactive protein may also be assessed in the patient prior to treatment and if elevated this may be used as a further indicator as to an increased risk of thrombosis in the patient.

For influenza-associated sepsis or septic shock, successful treatment may include a reduction in fever, a reduction in high or moderately-high heartbeat (e.g. tachycardia), a reduction in sweating (i.e. diaphoresis), decreased confusion and/or a decrease tiredness, and/or a decrease in shortness of breath, less labored or less rapid breathing. A treatment may be administered prophylactically, i.e., before the onset of sepsis or septic shock. A prophylactic treatment prevents sepsis or septic shock and can be administered to patients that have or are suspected of having an influenza infection, but without the severe symptoms of sepsis or septic shock. For example, prophylactic treatment can be administered to patients that have a cough without the other symptoms of sepsis or septic shock.

Additionally, the solid forms of Compound 1, or compositions thereof, may be used to treat sickle cell disease, particularly to reduce immunological responses that manifest in the disease. In some embodiments, the subject may exhibiting one or more of the following symptoms: anemia, sickle cell crisis, vaso-occlusive crisis, splenic sequestration crisis, splenic sequestration crises, acute chest syndrome, acute chest syndrome, aplastic crisis, hemolytic crisis, dactylitis, pneumonia, respiratory infection, bone-marrow embolization, or atelectasis.

Sickle cell disease (SCD) is a group of blood disorders typically inherited. The most common type is known as sickle cell anemia, which results in an abnormality in the oxygen carrying protein hemoglobin found in red blood cells. This leads to a rigid, sickle-like shape under certain circumstances. Problems in sickle cell disease typically begin around 5 to 6 months of age and a number of health problems may develop, such as attacks of pain (known as a sickle cell crisis), anemia, swelling in the hands and feet, bacterial infections and stroke. Long-term pain may develop as people get older.

Sickle cell disease occurs when a person inherits two abnormal copies of the β-globin gene (HBB) that makes hemoglobin, one from each parent. That gene occurs in chromosome 11. Several subtypes exist, depending on the exact mutation in each hemoglobin gene. An attack can be set off by temperature changes, stress, dehydration, and high altitude.

The care of people with sickle cell disease may include infection prevention with vaccination and antibiotics, high fluid intake, folic acid supplementation, and pain medication. Other measures may include blood transfusion and the medication hydroxycarbamide (hydroxyurea). A small percentage of people can be cured by a transplant of bone marrow cells. Patients with sickle cell disease may exhibit the following symptoms:

Sickle cell crisis: The terms “sickle cell crisis” or “sickling crisis” may be used to describe several independent acute conditions occurring in subjects with SCD, which results in anemia and crises that could be of many types, including the vaso-occlusive crisis, aplastic crisis, splenic sequestration crisis, hemolytic crisis, and others. Most episodes of sickle cell crises last between five and seven days. Although infection, dehydration, and acidosis (all of which favor sickling) can act as triggers, in most instances, no predisposing cause is identified.

Vaso-occlusive crisis: The vaso-occlusive crisis is caused by sickle-shaped red blood cells that obstruct capillaries and restrict blood flow to an organ, resulting in ischaemia, pain, necrosis, and often organ damage. The frequency, severity, and duration of these crises vary considerably. Painful crises are treated with hydration, analgesics, and blood transfusion; pain management requires opioid drug administration at regular intervals until the crisis has settled. For milder crises, a subgroup of subjects manages on nonsteroidal anti-inflammatory drugs such as diclofenac or naproxen. For more severe crises, most subjects require in-subject management for intravenous opioids; subject-controlled analgesia devices are commonly used in this setting. Vaso-occlusive crisis involving organs such as the penis or lungs are considered an emergency and treated with red blood cell transfusions. Incentive spirometry, a technique to encourage deep breathing to minimize the development of atelectasis, is recommended.

Splenic sequestration crisis: The spleen is frequently affected in sickle cell disease, as the sickle-shaped red blood cells cause narrowing of blood vessels and reduced function in clearing the defective cells. It is usually infarcted before the end of childhood in individuals with sickle cell anemia. This spleen damage increases the risk of infection from encapsulated organisms; preventive antibiotics and vaccinations are recommended for those lacking proper spleen function.

Splenic sequestration crises are acute, painful enlargements of the spleen, caused by intrasplenic trapping of red cells and resulting in a precipitous fall in hemoglobin levels with the potential for hypovolemic shock. Sequestration crises are considered an emergency. If not treated, subjects may die within 1-2 hours due to circulatory failure. Management is supportive, sometimes with blood transfusion. These crises are transient; they continue for 3-4 hours and may last for one day.

Acute chest syndrome: Acute chest syndrome is defined by at least two of these signs or symptoms: chest pain, fever, pulmonary infiltrate or focal abnormality, respiratory symptoms, or hypoxemia. It is the second-most common complication and it accounts for about 25% of deaths in subjects with SCD. Most cases present with vaso-occlusive crises, and then develop acute chest syndrome. Nevertheless, about 80% of people have vaso-occlusive crises during acute chest syndrome.

Aplastic crisis: Aplastic crises are instances of an acute worsening of the subject's baseline anemia, producing pale appearance, fast heart rate, and fatigue. This crisis is normally triggered by parvovirus B19, which directly affects production of red blood cells by invading the red cell precursors and multiplying in and destroying them. Parvovirus infection almost completely prevents red blood cell production for two to three days. In normal individuals, this is of little consequence, but the shortened red cell life of SCD subjects results in an abrupt, life-threatening situation. Reticulocyte counts drop dramatically during the disease (causing reticulocytopenia), and the rapid turnover of red cells leads to the drop in hemoglobin. This crisis takes 4 to 7 days to disappear. Most subjects can be managed supportively; some need a blood transfusion.

Hemolytic crisis: Hemolytic crises are acute accelerated drops in hemoglobin level. The red blood cells break down at a faster rate. This is particularly common in people with coexistent G6PD deficiency. Another influence of hemolytic crises in Sickle Cell Disease is oxidative stress on the erythrocytes, leukocytes, and platelets. When there is not enough red blood cell production in the bone marrow, the oxygen that the body receives, processes, and transports is unbalanced with the body's antioxidants. There is an imbalance in the oxygen reactive species in the cells, which leads to more production of red blood cells that are not properly oxygenated or formed. Oxidative stress may lead to anemia because of the imbalance of oxygen in the tissue. Management is supportive, sometimes with blood transfusions.

In addition, one of the earliest clinical manifestations is dactylitis, presenting as early as six months of age, and may occur in children with sickle cell trait. The crisis can last up to a month. Given that pneumonia and sickling in the lung can both produce symptoms of acute chest syndrome, the subject is treated for both conditions. It can be triggered by painful crisis, respiratory infection, bone-marrow embolization, or possibly by atelectasis, opiate administration, or surgery. Hematopoietic ulcers may also occur.

Additionally, the pharmaceutical compositions provided herein may be used to treat a lung injury. The lung injury may be a chemical- or radiation-induced lung injury.

In some embodiments, the subject may have inhaled or may be expected to be exposed to a pulmonary irritant. In some embodiments, the subject may have inhaled or may be expected to inhale a choking agent. A pulmonary agent, or choking agent, is a chemical agent designed to impede a subject's ability to breathe. These compounds generally operate by causing a build-up of fluids in the lungs, which then leads to suffocation. Inhalation of these agents cause burning of the throat, coughing, vomiting, headache, pain in chest, tightness in chest, and respiratory and circulatory failure. Examples of such agents include: chlorine gas, chloropicrin (PS), diphosgene (DP), phosgene (CG), disulfur decafluoride, perfluoroisobutene, acrolein, and piphenylcyanoarsine. Phosgene-induced acute lung injury (P-ALI) is commonly associated with short-term phosgene inhalation. Prolonged exposure can cause chronic hypoventilation, refractory pulmonary edema, and other associated lung injuries, ultimately resulting in ARDS. Chemical pneumonitis is inflammation of the lungs or breathing difficulty due to inhaling chemical fumes or breathing in and choking on certain chemicals.

Additionally, the solid forms of Compound 1, or compositions thereof, may be used to treat or prevent acute inhalation injury (All) and e-cigarette, or vaping, product use-associated lung injury (EVALI).

In other embodiments, the subject has been exposed to or is expected to be exposed to ionizing radiation. In these embodiments, the subject may have or may be expected to develop radiation induced lung injury (RILI). In some embodiments, the subject may have radiation pneumonitis or radiation pulmonary fibrosis. In these embodiments, the subject may have received or is undergoing thoracic radiotherapy, may have inhaled a radioactive agent or may have had direct exposure to ionizing radiation. For example, the subject may have inhaled a radioactive agent or have had direct exposure to ionizing radiation as a result of a nuclear weapon or leak at a nuclear power plant, for example.

The pharmaceutical compositions provided herein also may be used to treat or prevent hemorrhagic fever, or symptoms thereof, including Ebola virus disease, Alkhurma hemorrhagic fever, Chapare hemorrhagic fever, Crimean-Congo hemorrhagic fever, Hantavirus Pulmonary Syndrome (UPS), Hemorrhagic fever with renal syndrome (HFRS), Kyasanur Forest Disease (KFD), Lassa fever, Lujo hemorrhagic fever, Marburg hemorrhagic fever, Omsk hemorrhagic fever, Rift Valley fever, Yellow Fever, or Dengue fever, such as severe dengue fever (dengue hemorrhagic fever).

In one embodiment Compound 1 or the tablet composition comprising Compound 1 is for use in any of the above described indications.

### 7. Combination Therapy

In some embodiments, the pharmaceutical compositions provided herein are administered with another therapeutic agent, such as an analgesic, an antibiotic, an anticoagulant, an antibody, an anti-inflammatory agent, an immunosuppressant, a guanylate cyclase-C agonist, an intestinal secretagogue, an antiviral, anticancer, antifungal, or a combination thereof. In certain embodiments, the second therapeutic is an anti-inflammatory agent, an immunosuppressant and/or may be a steroid. In certain embodiments, the second therapeutic is a hypomethylating agent, an immunomodulatory imide drug or a mutant IDH1 inhibitor.

These various agents can be used in accordance with their standard or common dosages, as specified in the prescribing information accompanying commercially available forms of the drugs (see also, the prescribing information in the 2006 Edition of The Physician's Desk Reference), the disclosures of which are incorporated herein by reference.

### ENUMERATED EMBODIMENTS

Embodiment 1: A pharmaceutical composition comprising:
a. 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate having the following formula:

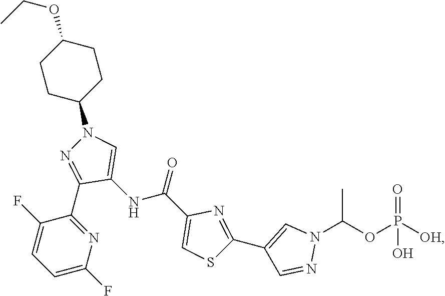 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

or a stereoisomer or mixture of stereoisomers, a pharmaceutically acceptable salt, tautomer, solvate, hydrate, co-crystal, clathrate, or polymorph thereof (Compound 1), wherein Compound 1 is present in an amount from about 15% to about 35% by weight based on total weight of the pharmaceutical composition;
b. intra-granular excipients from about 50% to about 70% by weight based on total weight of the pharmaceutical composition; and
c. extra-granular excipients from about 10% to about 20% by weight based on total weight of the pharmaceutical composition.
Embodiment 2: The pharmaceutical composition of Embodiment 1, wherein Compound 1 is 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt having the following formula:

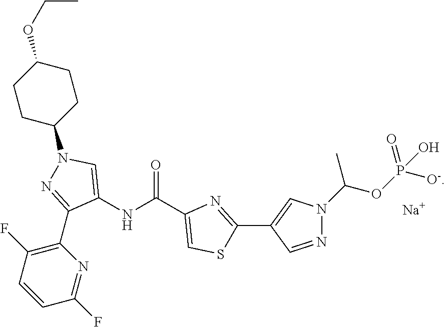

Embodiment 3: The pharmaceutical composition of Embodiment 1, wherein Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate having the following formula:

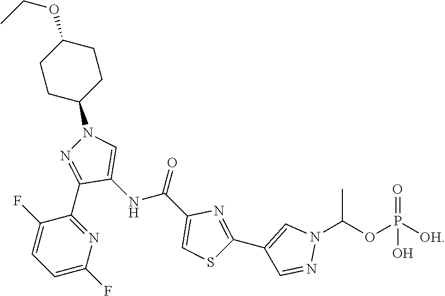 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

Embodiment 4: The pharmaceutical composition of any one of Embodiment 1 to 3, wherein the intra-granular excipients comprise one or more of a diluent, a disintegrant, a glidant and a lubricant.
Embodiment 5: The pharmaceutical composition of Embodiment 4, wherein the diluent comprises one or more of microcrystalline cellulose and mannitol.
Embodiment 6: The pharmaceutical composition of Embodiment 5, wherein the diluent comprises about 25% to about 35% microcrystalline cellulose by weight based on total weight of the pharmaceutical composition.
Embodiment 7: The pharmaceutical composition of any one of Embodiments 4 to 6, wherein the diluent comprises about 28%, about 28.92%, about 29%, about 29.25%, about 30% or about 32% microcrystalline cellulose by weight based on total weight of the pharmaceutical composition.
Embodiment 8: The pharmaceutical composition of any one of Embodiments 4 to 7, wherein the diluent comprises about 28.92% or about 29.25% microcrystalline cellulose by weight based on total weight of the pharmaceutical composition.
Embodiment 9: The pharmaceutical composition of Embodiment 4, wherein the diluent comprises mannitol.
Embodiment 10: The pharmaceutical composition of Embodiment 9, wherein the diluent comprises about 25% to about 35% mannitol by weight based on total weight of the pharmaceutical composition.
Embodiment 11: The pharmaceutical composition of Embodiment 9 or 10, wherein the diluent comprises about 28%, about 28.92%, about 29%, about 29.25%, about 30% or about 32% mannitol by weight based on total weight of the pharmaceutical composition.
Embodiment 12: The pharmaceutical composition of any one of Embodiments 9 to 11, wherein the diluent comprises about 28.92% or about 29.25% mannitol by weight based on total weight of the pharmaceutical composition.
Embodiment 13: The pharmaceutical composition of Embodiment 4, wherein the disintegrant comprises croscarmellose sodium.
Embodiment 14: The pharmaceutical composition of Embodiment 13, wherein the disintegrant comprises about 2% to about 4% croscarmellose sodium by weight based on total weight of the pharmaceutical composition.
Embodiment 15: The pharmaceutical composition of Embodiment 13 or 14, wherein the disintegrant comprises about 2.5%, about 3% or about 4% croscarmellose sodium by weight based on total weight of the pharmaceutical composition.
Embodiment 16: The pharmaceutical composition of any one of Embodiments 13 to 15, wherein the disintegrant comprises about 3% croscarmellose sodium by weight based on total weight of the pharmaceutical composition.
Embodiment 17: The pharmaceutical composition of Embodiment 4, wherein the glidant comprises fumed silica.
Embodiment 18: The pharmaceutical composition of Embodiment 17, wherein the glidant comprises about 0.5% to about 2% fumed silica by weight based on total weight of the pharmaceutical composition.
Embodiment 19: The pharmaceutical composition of Embodiment 18, wherein the glidant comprises about 0.5%, about 1% or about 2% fumed silica by weight based on total weight of the pharmaceutical composition.
Embodiment 20: The pharmaceutical composition of Embodiment 18 or 19, wherein the glidant comprises about 0.5% or about 1% fumed silica by weight based on total weight of the pharmaceutical composition.
Embodiment 21: The pharmaceutical composition of Embodiment 4, wherein the lubricant comprises sodium stearyl fumarate.
Embodiment 22: The tablet of Embodiment 21, wherein the lubricant comprises about 0.5% to about 2% sodium stearyl fumarate by weight based on total weight of the pharmaceutical composition.
Embodiment 23: The pharmaceutical composition of Embodiment 22, wherein the lubricant comprises about 0.5%, about 1% or about 2% sodium stearyl fumarate by weight based on total weight of the pharmaceutical composition.
Embodiment 24: The pharmaceutical composition of Embodiment 22 or 23, wherein the lubricant comprises about 1% sodium stearyl fumarate by weight based on total weight of the pharmaceutical composition.
Embodiment 25: The pharmaceutical composition of any one of Embodiments 1 to 24, wherein the extra-granular excipient comprises from a compression aid, a disintegrant, a glidant and a lubricant.
Embodiment 26: The pharmaceutical composition of Embodiment 25, wherein the compression aid comprises microcrystalline cellulose.
Embodiment 27: The pharmaceutical composition of Embodiment 26, wherein the compression aid comprises about 6% to about 10% microcrystalline cellulose by weight based on total weight of the pharmaceutical composition.
Embodiment 28: The pharmaceutical composition of Embodiment 25 or 27, wherein the compression aid comprises about 6%, about 7%, about 8% or about 10% microcrystalline cellulose by weight based on total weight of the pharmaceutical composition.
Embodiment 29: The pharmaceutical composition of any one of Embodiments 25 to 28, wherein the compression aid comprises about 7% or about 8% microcrystalline cellulose by weight based on total weight of the pharmaceutical composition.
Embodiment 30: The pharmaceutical composition of Embodiment 25, wherein the disintegrant comprises croscarmellose sodium.
Embodiment 31: The pharmaceutical composition of Embodiment 30, wherein the disintegrant comprises about 2% to about 4% croscarmellose sodium by weight based on total weight of the pharmaceutical composition.
Embodiment 32: The pharmaceutical composition of Embodiment 30 or 31, wherein the disintegrant comprises about 2.5%, about 3% or about 4% croscarmellose sodium by weight based on total weight of the pharmaceutical composition.
Embodiment 33: The pharmaceutical composition of any one of Embodiments 30 to 32, wherein the disintegrant comprises about 3% croscarmellose sodium by weight based on total weight of the pharmaceutical composition.
Embodiment 34: The pharmaceutical composition of Embodiment 25, wherein the lubricant comprises sodium stearyl fumarate.
Embodiment 35: The pharmaceutical composition of Embodiment 34, wherein the lubricant comprises about 1% to about 3% sodium stearyl fumarate by weight based on total weight of the pharmaceutical composition.
Embodiment 36: The pharmaceutical composition of Embodiment 34 or 35, wherein the lubricant comprises about 2% sodium stearyl fumarate by weight based on total weight of the pharmaceutical composition.
Embodiment 37: A tablet comprising the pharmaceutical composition of any one of Embodiments 1 to 36.
Embodiment 38: The tablet of Embodiment 37, wherein the tablet comprises about 100 mg to about 300 mg Compound 1.
Embodiment 39: The tablet of Embodiment 37 or 38, wherein the tablet comprises about 150 mg or about 250 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 40: The tablet of Embodiment 37 or 38, wherein the tablet comprises about 154 mg or about 269.39 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.
Embodiment 41: The tablet of any one of Embodiment 37 or 38, wherein the tablet comprises:
about 269.39 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 283.20 mg microcrystalline cellulose, about 282.50 mg mannitol, about 30 mg croscarmellose sodium, about 5 mg fumed silica, and about 10 mg sodium stearyl fumarate; and
the extra-granular excipient comprising 70 mg microcrystalline cellulose, about 30 mg croscarmellose sodium, and about 20 mg sodium stearyl fumarate.
Embodiment 42: The tablet of any one of Embodiment 37 or 38, wherein the tablet comprises:
about 250 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 292.50 mg microcrystalline cellulose, about 292.50 mg mannitol, about 30 mg croscarmellose sodium, about 5 mg fumed silica, and about 10 mg sodium stearyl fumarate; and
the extra-granular excipient comprising 70 mg microcrystalline cellulose, about 30 mg croscarmellose sodium, and about 20 mg sodium stearyl fumarate.
Embodiment 43: The tablet of any one of Embodiment 37 or 38, wherein the tablet comprises:
about 154 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 173.47 mg microcrystalline cellulose, about 173.47 mg mannitol, about 18 mg croscarmellose sodium, about 3 mg fumed silica, and about 6 mg sodium stearyl fumarate; and
the extra-granular excipient comprising 42 mg microcrystalline cellulose, about 18 mg croscarmellose sodium, and about 12 mg sodium stearyl fumarate.
Embodiment 44: The tablet of any one of Embodiment 37 or 38, wherein the tablet comprises:
about 150 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 175.50 mg microcrystalline cellulose, about 175.50 mg mannitol, about 18 mg croscarmellose sodium, about 3 mg fumed silica, and about 6 mg sodium stearyl fumarate; and
the extra-granular excipient comprising 42 mg microcrystalline cellulose, about 18 mg croscarmellose sodium, and about 12 mg sodium stearyl fumarate.
Embodiment 45: A pharmaceutical composition comprising:
   a. 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate having the following formula:

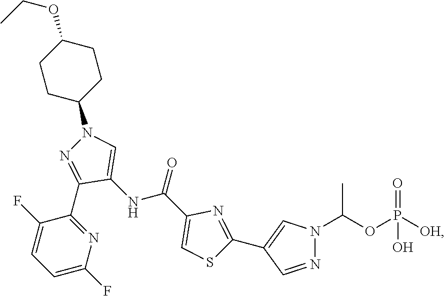 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

or a stereoisomer or mixture of stereoisomers, a pharmaceutically acceptable salt, tautomer, solvate, hydrate, co-crystal, clathrate, or polymorph thereof (Compound 1), wherein Compound 1 is present in an amount from about 65% to about 85% by weight based on total weight of the pharmaceutical composition;
b. intra-granular excipients from about 15% to about 20% by weight based on total weight of the pharmaceutical composition; and
c. extra-granular excipients from about 10% to about 15% by weight based on total weight of the pharmaceutical composition.
Embodiment 46: The pharmaceutical composition of Embodiment 45, comprising from about 65% to about 75% Compound 1 by weight based on total weight of the pharmaceutical composition.
Embodiment 47: The pharmaceutical composition of Embodiment 45 or 46, comprising about 65%, about 68%, about 70%, about 72% or about 75% Compound 1 by weight based on total weight of the pharmaceutical composition.
Embodiment 48: The pharmaceutical composition of any one of Embodiment 45 to 47, comprising about 65% or about 70% Compound 1 by weight based on total weight of the pharmaceutical composition.
Embodiment 49: A tablet comprising the pharmaceutical composition of any one of Embodiment 45 to 48.
Embodiment 50: The tablet of Embodiment 49 comprising:
a) about 60% to about 75% Compound 1 by weight based on total weight of the tablet;
b) an intra-granular excipient comprising about 4% to about 10% microcrystalline cellulose by weight based on total weight of the tablet, about 4% to about 10% mannitol by weight based on total weight of the tablet, about 2% to about 4% croscarmellose sodium by weight based on total weight of the tablet, about 0.5% to about 2% fumed silica by weight based on total weight of the tablet, and about 0.5% to about 2% sodium stearyl fumarate by weight based on total weight of the tablet; and
c) an extra-granular excipient comprising about 6% to about 10% microcrystalline cellulose by weight based on total weight of the tablet, about 2% to about 4% croscarmellose sodium by weight based on total weight of the tablet, and about 1% to about 3% sodium stearyl fumarate by weight based on total weight of the tablet.
Embodiment 51: The tablet of Embodiment 49 or 50, wherein the tablet comprises
a) about 65% Compound 1 by weight based on total weight of the tablet;
b) the intra-granular excipient comprising about 8.5% microcrystalline cellulose by weight based on total weight of the tablet, about 8.5% mannitol by weight based on total weight of the tablet, about 3% croscarmellose sodium by weight based on total weight of the tablet, about 1% fumed silica by weight based on total weight of the tablet, and about 1% sodium stearyl fumarate by weight based on total weight of the tablet; and
c) the extra-granular excipient comprising about 8% microcrystalline cellulose by weight based on total weight of the tablet, about 3% croscarmellose sodium by weight based on total weight of the tablet, and about 2% sodium stearyl fumarate by weight based on total weight of the tablet.
Embodiment 52: The tablet of Embodiment 49 or 50, wherein the tablet comprises
a) about 70% Compound 1 by weight based on total weight of the tablet;
b) the intra-granular excipient comprising about 6% microcrystalline cellulose by weight based on total weight of the tablet, about 6% mannitol by weight based on total weight of the tablet, about 3% croscarmellose sodium by weight based on total weight of the tablet, about 1% fumed silica by weight based on total weight of the tablet, and about 1% sodium stearyl fumarate by weight based on total weight of the tablet; and
c) the extra-granular excipient comprising about 8% microcrystalline cellulose by weight based on total weight of the tablet, about 3% croscarmellose sodium by weight based on total weight of the tablet, and about 2% sodium stearyl fumarate by weight based on total weight of the tablet.
Embodiment 53: The tablet of any one of Embodiments 49 to 52, wherein the tablet comprises about 100 mg to 1000 mg Compound 1.
Embodiment 54: The tablet of any one of Embodiments 49 to 53, wherein the tablet comprises about 250 mg to about 1000 mg Compound 1.
Embodiment 55: The tablet of any one of Embodiments 49 to 54, wherein the tablet comprises about 250 mg, about 500 mg, about 750 mg or about 1000 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 56: The tablet of any one of Embodiments 49 to 54, wherein the tablet comprises about 269.40 mg, about 538.79 mg or about 808.19 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.
Embodiment 57: The tablet of any one of Embodiments 49 to 54, wherein the tablet comprises:
a) about 269.40 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 23.09 mg microcrystalline cellulose, about 23.09 mg mannitol, about 11.55 mg croscarmellose sodium, about 3.85 mg fumed silica, and about 3.85 mg sodium stearyl fumarate; and
c) the extra-granular excipient comprising 30.79 mg microcrystalline cellulose, about 11.55 mg croscarmellose sodium, and about 7.70 mg sodium stearyl fumarate.
Embodiment 58: The tablet of any one of Embodiments 49 to 54, wherein the tablet comprises:
a) about 250 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 32.79 mg microcrystalline cellulose, about 32.79 mg mannitol, about 11.55 mg croscarmellose sodium, about 3.85 mg fumed silica, and about 3.85 mg sodium stearyl fumarate; and
c) the extra-granular excipient comprising 30.79 mg microcrystalline cellulose, about 11.55 mg croscarmellose sodium, and about 7.70 mg sodium stearyl fumarate.
Embodiment 59: The tablet of any one of Embodiments 49 to 54, wherein the tablet comprises:
a) about 538.79 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 46.18 mg microcrystalline cellulose, about 46.18 mg mannitol, about 23.09 mg croscarmellose sodium, about 7.70 mg fumed silica, and about 7.70 mg sodium stearyl fumarate; and
c) the extra-granular excipient comprising 61.58 mg microcrystalline cellulose, about 23.09 mg croscarmellose sodium, and about 15.39 mg sodium stearyl fumarate.
Embodiment 60: The tablet of any one of Embodiments 49 to 54, wherein the tablet comprises:
a) about 500 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 65.58 mg microcrystalline cellulose, about 65.58 mg mannitol, about 23.09 mg croscarmellose sodium, about 7.70 mg fumed silica, and about 7.70 mg sodium stearyl fumarate; and
c) the extra-granular excipient comprising 61.58 mg microcrystalline cellulose, about 23.09 mg croscarmellose sodium, and about 15.39 mg sodium stearyl fumarate.
Embodiment 61: The tablet of any one of Embodiments 49 to 54, wherein the tablet comprises:
a) about 808.19 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 69.27 mg microcrystalline cellulose, about 69.27 mg mannitol, about 34.64 mg croscarmellose sodium, about 11.55 mg fumed silica, and about 11.55 mg sodium stearyl fumarate; and
c) the extra-granular excipient comprising 92.36 mg microcrystalline cellulose, about 34.64 mg croscarmellose sodium, and about 23.09 mg sodium stearyl fumarate.
Embodiment 62: The tablet of any one of Embodiments 49 to 54, wherein the tablet comprises:
a) about 750 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 98.37 mg microcrystalline cellulose, about 98.37 mg mannitol, about 34.64 mg croscarmellose sodium, about 11.55 mg fumed silica, and about 11.55 mg sodium stearyl fumarate; and
c) the extra-granular excipient comprising 92.36 mg microcrystalline cellulose, about 34.64 mg croscarmellose sodium, and about 23.09 mg sodium stearyl fumarate.
Embodiment 63: The tablet of any one of Embodiments 49 to 54, wherein the tablet comprises:
a) about 1077.58 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 92.37 mg microcrystalline cellulose, about 92.37 mg mannitol, about 46.18 mg croscarmellose sodium, about 15.39 mg fumed silica, and about 15.39 mg sodium stearyl fumarate; and
c) the extra-granular excipient comprising 123.15 mg microcrystalline cellulose, about 46.18 mg croscarmellose sodium, and about 30.79 mg sodium stearyl fumarate.
Embodiment 64: The tablet of any one of Embodiments 49 to 54, wherein the tablet comprises:
a) about 999.99 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) the intra-granular excipients comprising about 131.16 mg microcrystalline cellulose, about 131.16 mg mannitol, about 46.18 mg croscarmellose sodium, about 15.39 mg fumed silica, and about 15.39 mg sodium stearyl fumarate; and
c) the extra-granular excipient comprising 123.15 mg microcrystalline cellulose, about 46.18 mg croscarmellose sodium, and about 30.79 mg sodium stearyl fumarate.
Embodiment 65: A method for preparing the tablet of any one of Embodiments 37 to 44 and 49 to 64, wherein the method comprises blending Compound 1 with an intra-granular excipient and an extra-granular excipient.
Embodiment 66: The method of Embodiment 65, wherein the method comprises blending Compound 1 with an intra-granular excipient to obtain a blend.
Embodiment 67: The method of Embodiment 66, further comprising roller compacting the blend to obtain a ribbon, milling the ribbon to form granules, and blending the granules with extra-granular excipients to obtain a final product blend.
Embodiment 68: The method of Embodiment 67 further comprising compressing the final product blend to obtain a tablet.
Embodiment 69: The method of any one of Embodiments 65 to 68, wherein the intra-granular excipient comprises microcrystalline cellulose, mannitol, croscarmellose sodium, fumed silica and sodium stearyl fumarate.
Embodiment 70: The method of any one of Embodiments 65 to 68, wherein the extra-granular excipient comprises microcrystalline cellulose, croscarmellose sodium and sodium stearyl fumarate.
Embodiment 71: A method of treating a disease comprising administering to a subject in need thereof the pharmaceutical composition of any one of Embodiments 1 to 36 and 45 to 48 or the tablet of any one of Embodiments 37 to 44 and 49 to 54, wherein the disease is an auto-immune disease, an inflammatory disorder, a cardiovascular disease, a nerve disorder, a neurodegenerative disorder, an allergic disorder, asthma, pancreatitis, multi-organ failure, a kidney disease, platelet aggregation, cancer, transplantation, sperm motility, erythrocyte deficiency, graft rejection, a lung injury, a respiratory disease, an ischemic condition, bacterial infection or a viral infection.
Embodiment 72: The method of Embodiment 71, wherein disease is an allergic disease, amyotrophic lateral sclerosis (ALS), systemic lupus erythematosus, rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmyopathy, or asthma.
Embodiment 73: The method of Embodiment 71, wherein disease is transplantation of organs or tissue, graft-versus-host diseases brought about by transplantation, autoimmune syndromes including rheumatoid arthritis, lupus, including systemic lupus erythematosus, Hashimoto's thyroiditis, multiple sclerosis, systemic sclerosis, myasthenia gravis, type I diabetes, uveitis, posterior uveitis, allergic encephalomyelitis, glomerulonephritis, postinfectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, celiac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, erythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA bullous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C4 release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic liver disease, including alcoholic cirrhosis, non-alcoholic steatohepatitis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, Parkinson's disease, trauma, chronic bacterial infection, palmoplantar pustulosis, hidradenitis suppurativa, cytokine release syndrome, acute respiratory distress syndrome, acute kidney injury, kidney malfunction, or thrombosis.
Embodiment 74: The method of Embodiment 71, wherein disease is hidradenitis suppurativa, or a lymphoid neoplasm selected from myeloproliferative neoplasms, myeloid/lymphoid neoplasms with PDGFRA rearrangement, myeloid/lymphoid neoplasms with PDGFRB rearrangement, myeloid/lymphoid neoplasms with FGFR1 rearrangement, myeloid/lymphoid neoplasms with PCM1-JAK2, myelodysplastic/myeloproliferative neoplasms, myeloid sarcoma, myeloid proliferations related to Down syndrome, blastic plasmacytoid dendritic cell neoplasm, B-lymphoblastic leukemia/lymphoma; and/or T-lymphoblastic leukemia/lymphoma.
Embodiment 75: The method of Embodiment 71, wherein disease is a myeloproliferative neoplasm selected from chronic myeloid leukemia, chronic neutrophilic leukemia, primary myelofibrosis, essential thrombocythemia, chronic eosinophilic leukemia, or a combination thereof.
Embodiment 76: The method of Embodiment 71, wherein disease is a myelodysplastic/myeloproliferative neoplasm selected from chronic myelomonocytic leukemia, atypical chronic myeloid leukemia, juvenile myelomonocytic leukemia, MDS/MPN with ring sideroblasts and thrombocytosis, or a combination thereof.
Embodiment 77: The method of Embodiment 71, wherein disease is arthralgia, arthritis, rheumatoid arthritis, psoriatic arthritis, osteoarthritis, systemic lupus erythematosus, lupus nephritis, ankylosing spondylitis, osteoporosis, systemic sclerosis, multiple sclerosis, psoriasis, in particular pustular psoriasis, type I diabetes, type II diabetes, inflammatory bowel disease, hyperimmunoglobulinemia d and periodic fever syndrome, cryopyrin-associated periodic syndromes, Schnitzler's syndrome, systemic juvenile idiopathic arthritis, adult's onset Still's disease, gout, gout flares, pseudogout, SAPHO syndrome, Castleman's disease, sepsis, stroke, atherosclerosis, celiac disease, deficiency of Il-1 receptor antagonist, Alzheimer's disease, or Parkinson's disease.
Embodiment 78: The method of Embodiment 71, wherein disease is a solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, IL-1 driven disorders, a MyD88 driven disorder, primary cutaneous T-cell lymphoma, chronic lymphocytic leukemia, smoldering or indolent multiple myeloma, leukemia, acute myeloid leukemia, DLBCL, ABC DLBCL, chronic lymphocytic leukemia, chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndrome, clonal cytopenia of undetermined significance, myelofibrosis, polycythemia vera, Kaposi's sarcoma, Waldenstrom's macroglobulinemia, splenic marginal zone lymphoma, multiple myeloma, plasmacytoma or intravascular large B-cell lymphoma.
Embodiment 79: The method of Embodiment 78, wherein disease is a myelodysplastic syndrome.
Embodiment 80: The method of Embodiment 79, wherein the myelodysplastic syndrome is a higher-risk myelodysplastic or a lower-risk myelodysplastic syndrome.
Embodiment 81: The method of Embodiment 79 or 80, wherein the myelodysplastic syndrome is refractory or relapsed myelodysplastic syndrome.
Embodiment 82: The method of any one of Embodiments 71 to 81, wherein the method further comprises administering a second therapeutic agent.

### EXAMPLES

The embodiments described below are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

Compound 1A can be prepared by methods known in the art, for example, U.S. Pat. No. 11,370,787 B2, incorporated by reference in its entirety. An exemplary method of preparation is described in Example 1.

### Example 1: Synthesis of-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium

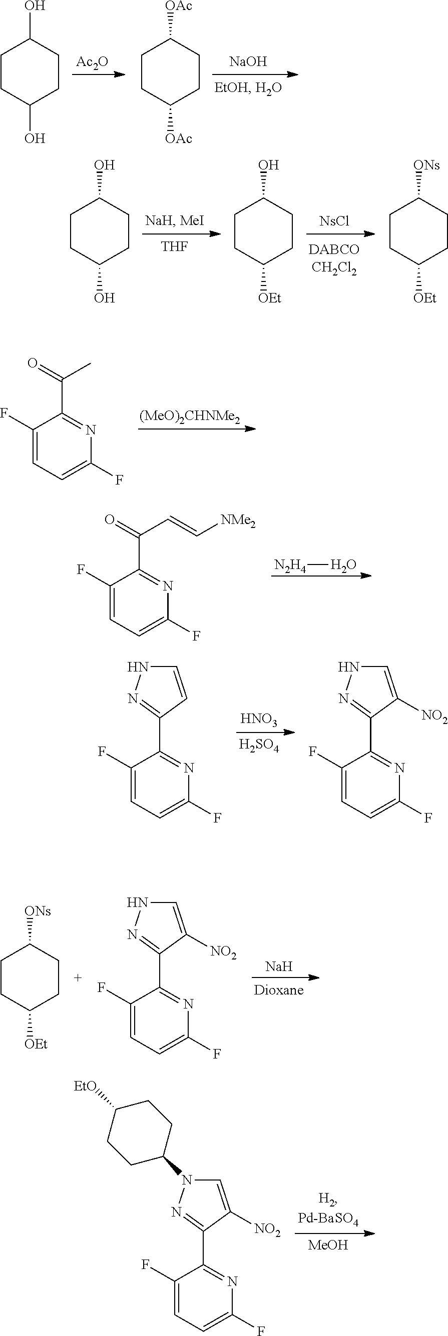

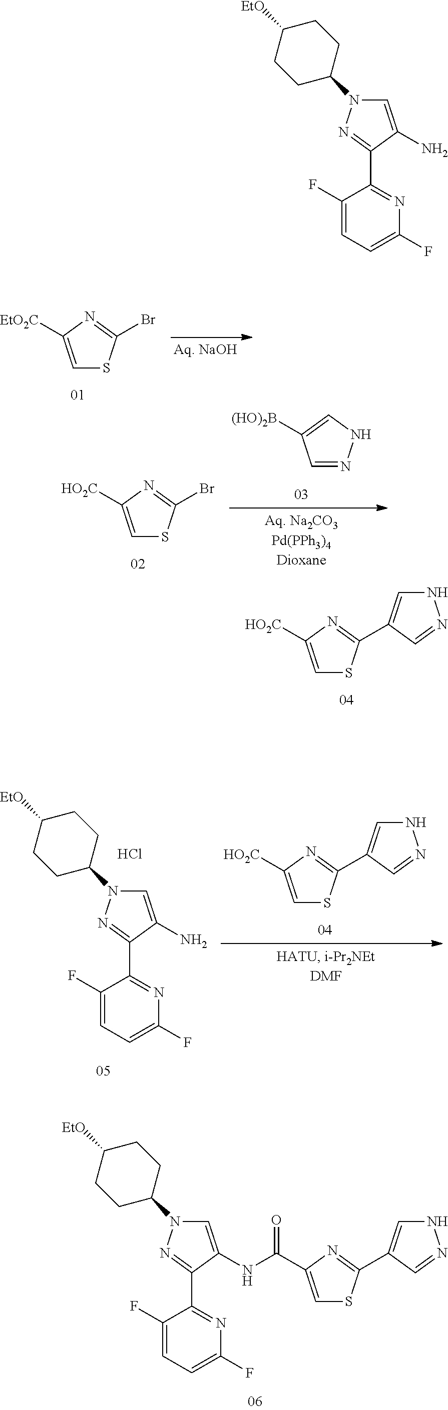

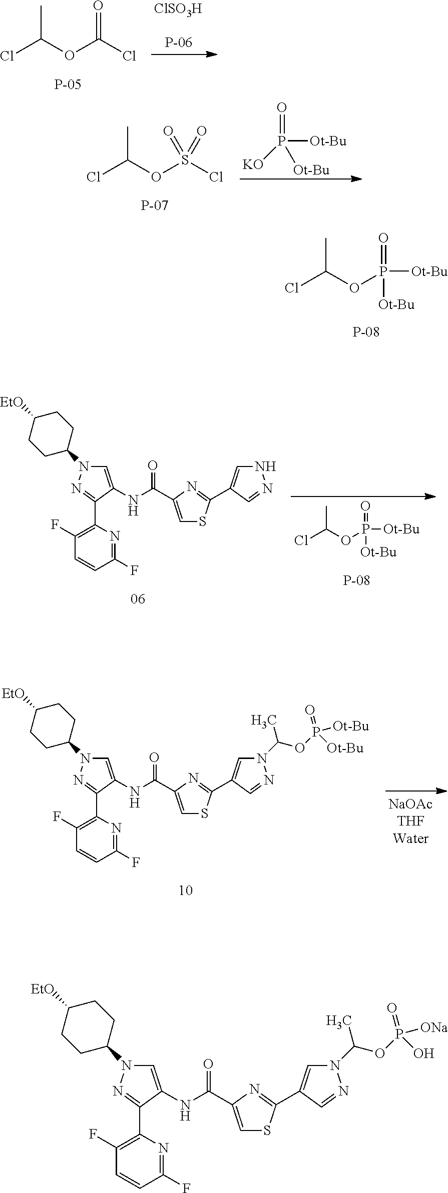

### Building Block A: N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (Compound-06) parent compound

### Step 1: Saponification

Compound 01 is saponified in an aqueous solution of NaOH at 10° C. to 20° C. The work-up is performed with HCl, the material is then filtered and washed with water and dried to yield Compound 02.

### Step 2: Suzuki Coupling

Compound 02, Compound 03, and Na₂CO₃ are added to a mixture of dioxane and water. Tetrakis (triphenylphosphine) palladium is added as a catalyst, and the reaction mixture is heated to 40° C. to 50° C. After a polish filtration, the solution is cooled and work-up is performed with HCl to precipitate Compound 04, which is filtered and washed with water and isopropanol before drying.

### Step 3: Amide Coupling

Compound 04 is suspended in dimethylformamide. N,N-Diisopropylethylamine, HOBt, and EDCI HCl are added followed by Compound 05 and the reaction mixture is stirred at room temperature. After a polish filtration, crude N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (Compound 06) is precipitated by adding the reaction mixture to an aqueous solution of sodium bicarbonate, washed with water and dried.

### Building Block B: Chloroethyl di-tert-butyl phosphate (P-08) prodrug moiety

### Step 1: Coupling/Substitution

Chlorosulphonic acid (P-06) is added slowly to neat chloroethyl chloroformate (P-05) and stirred at approximately −5° C. to 0° C. Extractive work-up is performed with dichloromethane and water, using sodium hydroxide to neutralize the acid. The dichloromethane layer is washed with sodium bicarbonate and sodium chloride solutions, and the aqueous layer is discarded to yield an approximately 15% to 25% w/w solution of chloroethyl chlorosulphate (P-07) in dichloromethane, used directly in the following step.

### Step 2: Coupling/Substitution

Potassium di-tert-butyl phosphate (P-02), sodium bicarbonate, tetrabutylammonium hydrogen sulfate, and water are charged to a reaction vessel and cooled to approximately 5° C. The P-07 solution in dichloromethane is chilled and added slowly to the reaction vessel. The mixture is stirred at 10° C. to 20° C. before extractive work-up by discarding the aqueous layer, washing with water and sodium chloride solution, discarding the aqueous layer again, and distilling off the dichloromethane solvent to yield a 75% to 85% w/w solution of chloroethyl di-tert-butyl phosphate (P-08) in dichloromethane.

### Addition of Prodrug Moiety to Parent Compound

### Step 1: Alkylation

N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (Compound 06), tetrabutylammonium iodide and cesium carbonate are added to a mixture of tetrahydrofuran and water. P-08 solution in dichloromethane is then added to the reaction mixture and heated to 60° C. to 70° C. The mixture is then cooled and filtered to remove inorganic salts before performing a solvent swap to ethyl acetate by distilling off tetrahydrofuran. tert-Butyl methyl ether (TBME) is then added to precipitate out Compound 10, which is filtered, washed with TBME, and dried.

### Step 2: Deprotection and Salt Formation

Compound 10 is dissolved in tetrahydrofuran and water, then an aqueous solution of sodium acetate is added, and the reaction mixture is heated to 55° C. to 65° C. Acetone is added to precipitate out 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt product, which is filtered, washed with acetone, and dried.

### Example 2: Preparation of Tablets

The following excipients and equipment were used in the preparation of tablets.

About 1000 g of Compound 1A were used for this study.

Potential excipients for tablet development and manufacturing were of compendial grade. A full list of excipients and equipment utilized for this study can be found in Table 1. In this study, percent compositions of solutions or solid dispersions are described on a weight:weight basis, unless otherwise specified.

TABLE 1
Materials and Equipment

| Material and Equipment | Trade Name | Abbreviation |
|---|---|---|
| Microcrystalline Cellulose | AVICEL ® PH-105 | MCC |
| Microcrystalline Cellulose | AVICEL ® PH-102 | MCC |
| Microcrystalline Cellulose | AVICEL ® PH-200 | MCC |
| Mannitol | PARTECK ® M100 | M100 |
| Mannitol | PEARLITOL ® 25C | PEARLITOL ®25 C |
| Croscarmellose Sodium | AC-DI-SOL ® | AC-DI-SOL ® |
| Untreated Fumed Colloidal Silicon | CAB-O-SIL ® M-5P | CAB-O-SIL ® |
| Dioxide | | |
| Sodium Stearyl Fumarate | PRUV ® SSF | SSF |
| Blender | | |
| Blender | | |
| 5 L Blender Shell | | |
| 15 L Blender Shell | | |
| Tablet Press | | |
| Tablet Tooling (0.8750″ FF) | | |
| Tablet Tooling (0.2500″ FF) | | |
| Tablet Tooling (0.3010″ × 0.6200″ | | |
| modified oval) | | |
| Tablet Tooling (0.3750″ × 0.7480″ | | |
| modified oval) | | |
| Roller Compactor | | |
| Granulator | | |
| Rotary Tablet Press | | |
| Compaction Simulator | | |
| Sieve Shaker | | |
| Hardness Tester | | |
| Disintegration Apparatus | | |
| Tapped density tester | | |
| Envelope Density Tester | | |
| True Density Tester | | |

### Methods

Compound 1A, powder blends, and tablets were characterized using one or more of the following analytical experiments: assay and impurities by high-performance liquid chromatography (HPLC), water content by Karl Fisher titration (KF), non-sink dissolution, bulk and tapped density, particle size distribution (PSD) by sieve analysis, and envelope density analysis to determine ribbon solid fraction.

### Water Content by Coulometric Karl Fisher (KF) Titration

Samples were analyzed for water content by a Metrohm 831 Karl Fischer Coulometric Titrator with a Metrohm 874 oven processor. About 65 mg samples were sealed in 6 mL crimp vials followed by measurement of water content with the following parameters: Reagent Hydranal Coulomat AG-Oven, Oven temperature 130° C. and sample extraction time 300 seconds per AM-0300-00.

### Assay and Impurities Analysis by HPLC

Assay and impurities of the samples were evaluated using two experimental HPLC methods (Table 2 and Table 3). The development method (DM) was used for early stage analysis and the analytical method (AM) was used for ASAP stability sample analysis. The methods demonstrated passing system suitability criteria for their respective development work, including but not limited to resolution, standard agreement, tailing, and signal to noise. The HPLC method described herein was performed as part of the GMP scope of the project, without significant changes to the method during validation.

TABLE 2
HPLC Parameters for Assay and Impurities, Analytical Method

| Parameter | Value | | |
|---|---|---|---|
| Column | Agilent ZORBAX Extend C18, 5 μm, 4.6 × | | |
| | 150 mm, PN: 773450-902 | | |
| Mobile Phase A | 20 mM NH₄OAc at pH 9.0 | | |
| Mobile Phase B | ACN | | |
| Diluent | 60:40 Water:THF | | |
| Gradient | Time | % Mobile | % Mobile |
| Program | (min) | Phase A | Phase B |
| | 0.0 | 80 | 20 |
| | 12.0 | 70 | 30 |
| | 22.0 | 50 | 50 |
| | 28.0 | 5 | 95 |
| | 30.0 | 5 | 95 |
| | 30.1 | 80 | 20 |
| | 36.0 | 80 | 20 |

| | |
|---|---|
| Flow Rate | 1.0 mL/min |
| Column | 35° C. |
| Temperature | |
| Sample | Room temperature |
| Temperature | |
| Injection Volume | 7 μL |
| Needle Wash | Diluent |
| Detection Method | UV |
| Detection | 278 nm |
| Wavelength | |
| Detection | 4 nm |
| Bandwidth | |
| Slit Width | 4 nm |
| Reference | Off |
| Wavelength | |
| Collect Spectra | 190-400 nm (Only required for ID analysis) |
| Run Time | 36.0 min |
| Compound 1A | 10.5 min |
| Approximate RT | |

TABLE 3
HPLC Parameters for Assay and Impurities, Development Method

| Parameter | Value | | |
|---|---|---|---|
| Column | Thermo Hypersil Gold C18, 50 × 2.1 mm, 1.9 μm, | | |
| | Part number: 25002-052130, Serial number: | | |
| | 20090839 | | |
| Mobile Phase A | 20 mM NH₄OAc at pH 9.0 | | |
| Mobile Phase B | ACN | | |
| Diluent | 1:1 THF:H₂O | | |
| Gradient | Time | % Mobile | % Mobile |
| Program | (min) | Phase A | Phase B |
| | 0.0 | 80 | 20 |
| | 0.7 | 70 | 30 |
| | 7.0 | 50 | 50 |
| | 11.7 | 5 | 95 |
| | 11.8 | 5 | 95 |
| | 16.0 | 80 | 20 |

| | |
|---|---|
| Flow Rate | 0.8 mL/min |
| Column | 40° C. |
| Temperature | |
| Sample | Room temperature |
| Temperature | |
| Injection Volume | 3 μL |
| Needle Wash | Diluent |
| Detection Method | UV |
| Detection | 278 nm |
| Wavelength | |
| Detection | 4 nm |
| Bandwidth | |
| Slit Width | 4 nm |
| Reference | Off |
| Wavelength | |
| Collect Spectra | 190-400 nm |
| Run Time | 16.0 min |
| Compound 1A | 3.7 min |
| Approximate RT | |

### Biorelevant Dissolution Performance

Biorelevant drug dissolution performance for tablets was evaluated by a two stage ‘gastric transfer’ non-sink dissolution test (Table 4), which simulates pH and bile salt concentrations for both gastric and intestinal exposure. Whole tablets were transferred to a preheated (37° C.) volume of 0.1N HCl (aq) pH˜1.0, without pepsin or bile salts), in a USP Type 2 vessel (1 L or 100 mL total vessel volume) while stirring (paddles) at 75 rpm for 1 L vessels and 100 rpm for 100 mL mini-vessels. After 30 minutes of gastric pH exposure, an equal volume of PBS buffered, 2× concentrated fasted-state simulated intestinal fluid (FaSSIF) was added to the HCl, resulting in a final pH of 6.8 in FaSSIF (100 mM PBS containing 2.24 mg/mL FaSSIF/FeSSIF/FaSSGF powder (Biorelevant Inc.) in a total volume of 100 mL. Aliquots (1.0 mL) of dissolution media were taken at the following time points: 10, 25, 35, 50, 70, 120 and 210 minutes. The first two aliquots were taken from the simulated gastric medium, with the remaining five aliquots from the intestinal medium. Aliquots were centrifuged at 13000 rpm to pellet out undissolved solids, and the supernatant was sampled and further diluted in an appropriate diluent to determine API total drug concentration (e.g. free and colloidal/polymer-bound drug in solution) utilizing a suitable HPLC method. The volume of FaSSIF added was adjusted to account for the sampling volume removed prior to gastric transfer (typically 4×1.0 mL). Initial 2 mg API concentration in dissolution samples was determined utilizing an HPLC method (Table 5).

TABLE 4
Non-Sink Dissolution Test Parameters

| Parameter | Value |
|---|---|
| Apparatus | USP Type 2 (1 L or 100 mL) |
| Gastric Media | 0.1N HCl (aq) |
| Intestinal Media | FaSSIF |
| Temperature | 37 ± 0.5° C. |
| Paddle Speed | 75 or 100 RPM |
| Dose | 2.0 → 1.0 mgA/mL (milligram active/milliliter) |

TABLE 5
HPLC Parameters for Non-Sink Dissolution Analysis Nm

| Parameter | Value | |
|---|---|---|
| Column | Waters Xterra, MS C18, 3.5 μm, 4.6 × 150 mm, | |
| | PN: 186000440, SN: 02363810714012 | |
| Mobile Phase A | 20 mM NH₄OAc buffer in water, pH 9.0 | |
| Mobile Phase B | Acetonitrile | |
| Diluent | 3:1 ACN:Water (v/v) | |
| Gradient Program | Time (min) | % MPB |
| | 0.00 | 25 |
| | 1.50 | 50 |
| | 4.00 | 90 |
| | 4.10 | 25 |
| | 5.00 | 25 |

| | |
|---|---|
| Flow Rate | 1.5 mL/min |
| Column Temperature | 40° C. |
| Sample Temperature | Room Temperature |
| Injection Volume | 8.0 μL |
| Needle Wash | Diluent |
| Detection Method | UV |
| Detection Wavelength | 280 nm |
| Detection Bandwidth | 4 nm |
| Slit Width | 4 nm |
| Reference Wavelength | Off |
| Collect Spectra | No |
| Run Time | 5 min |
| Compound 1A | 2.5 min |
| Approximate RT | |

### Preparation of Media

Gastric Media: Determine the volume of gastric medium needed for all dissolution samples. Based on this volume, dilute 1.0 N HCl 10× with Milli-Q H2O in a suitable Class A graduated cylinder or volumetric flask. RO water may be used for large volumes. Mix well, test approximate pH using calibrated pH meter. The observed pH should be 1.0-1.1.

PBS buffer (200 mM): Determine the volume of buffer needed for all dissolution samples. Based on this volume, weigh the appropriate amount of Na₂HPO₄ to reach 200 mMol/L NaCl and 200 mMol/L and transfer into an appropriately sized vessel. To this vessel, add the appropriate volume of H₂O. Magnetically stir the solution until all salts are fully dissolved. If necessary, adjust with at least 1.0N NaOH to pH 8.9±0.5.

2× concentrated FaSSIF Medium (4.48 mg/mL): To PBS medium above, add 4.48 mg FaSSIF/FeSSIF/FaSSGF powder (Biorelevant Inc.) per mL of 200 mM PBS. Mix well, stirring with a magnetic stir bar until all SIF is in solution. Let stand two hours at RT before use, and then preheat to 37° C. for the dissolution test. If 2× concentrated FaSSIF will not be used the day it is prepared, store at ambient conditions up to 48 hours. Equilibrate to 37° C. at least two hours before use.

### Bulk Density and Tapped Density Analysis

Tablet blends were evaluated for bulk and tapped density per “Tapped Density Method I”. A 100 mL glass cylinder along with corresponding base plate was used for all samples. An ERWEKA SVM Tapped Density Tester was utilized to perform analysis and tapped at a rate of 300 taps/minute. Hausner ratio and Carr index calculations can be found below in Equation 1, and a summary of flow classifications can be found in Table 6.

$$Hausner Ratio = \frac{Tapped Density}{Bulk Density}$$

$$Carr Index = 100 \frac{Tapped Density - Bulk Density}{Tapped Density}$$

### Equation 1. Equations for Calculating Hausner Ratio and Carr Index

TABLE 6
Summary of Flow Classification for
Hausner Ratio and Carr Index Values

| Hausner Ratio | Carr Index | Flow Classification |
|---|---|---|
| 1.00-1.11 | ≤10 | Excellent/very free flowing |
| 1.12-1.18 | 11-15 | Good/free flowing |
| 1.19-1.25 | 16-20 | Fair |
| 1.26-1.34 | 21-25 | Passable |
| 1.35-1.45 | 26-31 | Poor/cohesive |
| 1.46-1.59 | 32-37 | Very poor/very cohesive |
| >1.60 | >38 | Extremely poor |

### Tablet Friability

Tablet friability was determined by utilizing a Pharmatron FT 2 friability tester. A drum rotation speed of 25 rpm was used at a total rotation time of 4 minutes. Acceptable loss on friability per USP method is ≤1.0 weight percent.

### Disintegration

Disintegration was evaluated utilizing a Varian VK-100 disintegration apparatus. The apparatus consists of a 1000 mL low-form beaker and basket-rack assembly with six open-ended transparent tubes. The beaker contained 750 mL of RO water and was maintained at a temperature of 37° C. (±2° C.). The basket was fully submerged at a frequency of 29-32 cycles per minute and tablet disintegration time was recorded when the last visible tablet materials passed through the basket.

### Tablet Hardness and Tensile Strength

Tablet hardness was tested utilizing a Natoli Hardness Tester (S/N 1403029). Tablet thickness and weight were measured prior to assessing the tablet break force as it is a destructive process. Tablets were placed in the automated breaking apparatus and tablet hardness was measured in kilogram-force/kilopond (kp). Tensile strength for standard round concave (SRC) tablets and elongated tablets (modified oval) were calculated based on the following equations:

$$Tablet Tensile Strength ( SRC ) = \frac{10 P}{π {D}^{2} ( 2.84 \frac{t}{D} - 0.126 \frac{t}{W} + 3.15 \frac{W}{D} + 0.01 )}$$

$$Tablet Tensile Strength ( elongated tablet ) = \frac{2}{3} ( \frac{10 P}{π {D}^{2} ( 2.84 \frac{t}{D} - 0.126 \frac{t}{W} + 3.15 \frac{W}{D} + 0.01 )} )$$

Where P=fracture load, D=tablet width, t=tablet thickness, W=band thickness (K. G. Pitt and M. G. Heasley. “Determination of the tensile strength of elongated tablets.” Powder Technology, vol. 238 (2013) pp. 169-175.)

### Particle Size Distribution by Sieve Analysis

Particle size distribution was determined by an analytical sieving method. A sieve shaker was utilized to evaluate material. Screens utilized and operating parameters can be found below in Table 7.

TABLE 7
Equipment and Parameters for Particle Size Distribution
Analysis via Analytical Sieving Method

| Parameter | Value |
|---|---|
| Sieve Shaker | |
| Shaker Mode | Coarse |
| Operating Time | 5 min |
| Screen #1 | 18 mesh (1000 μm) |
| Screen #2 | 20 mesh (841 μm) |
| Screen #3 | 30 mesh (595 μm) |
| Screen #4 | 40 mesh (420 μm) |
| Screen #5 | 60 mesh (250 μm) |
| Screen #6 | 120 mesh (125 μm) |

### Envelope Density Analysis

Ribbons produced by the roller compactor were tested for envelope density using a GeoPyc 1360 (Micromeritics). Ribbons were accurately weighed using a balance and placed in a 19.1″ ID sample chamber with DryFlo medium. DryFlo was agitated and gently consolidated about the sample to a force of 38 N for n=3 cycles. The GeoPyc collected displacement data and performed calculations for envelope density. Ribbon solid fraction was calculated using the equation below, where Pe=envelope density and P0=true density, assuming a true density of 1.35 g/mL for the formulations.

$$Solid Fraction = \frac{{P}_{e}}{{P}_{0}}$$

### Compound Analysis and Property Assessment

### Aqueous Solubility

Solubility of bulk Compound 1A Na as received API was conducted in various biorelevant media. Small amounts of API were suspended in media and continuously agitated at 37° C. for a period up to 24 hours. Samples were centrifuged to pellet out undissolved solids and the resulting supernatant was sampled, diluted, and analyzed by HPLC utilizing the short-assay method used for dissolution sample analysis. Results are listed below in Table 8.

TABLE 8
Solubility of Bulk Compound 1A in
Biorelevant Media Measured by HPLC

| Media | Solubility (mg/mL) | Solubility (mg/mL) |
|---|---|---|
| 0.1N HCl | 0.01 | 0.00 |
| FaSSIF, pH 6.8 | 1.14 | 1.13 |

### Physical Characterization of Drug Substance

Compound 1A was also characterized by bulk density, true density, and compression profile. Table 9 below show bulk and true density values. The low bulk density value indicated that granulation will likely be necessary to achieve high dose tablets.

TABLE 9
Neat Compound 1A bulk and true density values

| Test | Value |
|---|---|
| Bulk Density (g/cc) | 0.10 |
| True Density (g/cc) | 1.21 |

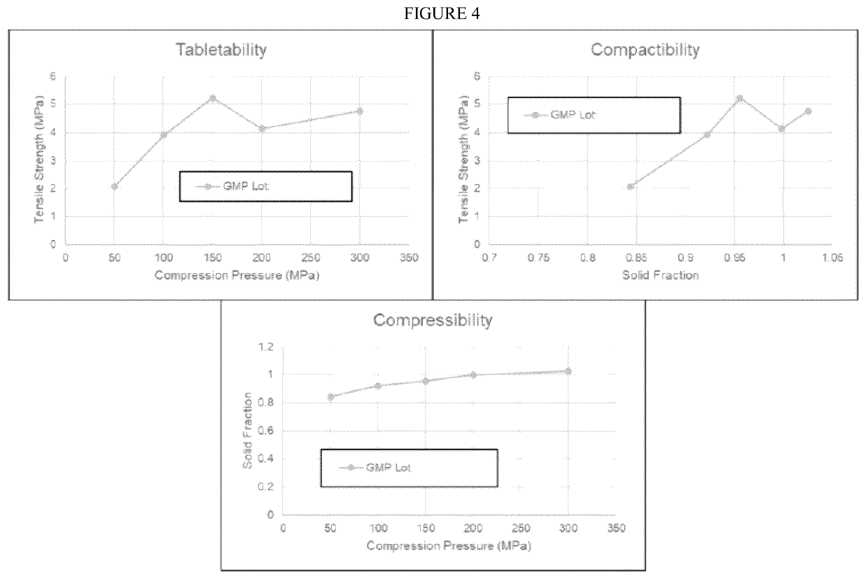

A compression profile of Compound 1A was generated using 0.25″ flat faced tooling on the Natoli single station tablet press. Compression pressure range of 50-300 MPa was evaluated and tabletability, compactibility and compressibility graphs were generated. Compression profiles show that neat Compound 1A is highly compressible where a tensile strength of 5 MPa was observed. The tabletabilty and compactability graphs indicate that over compression may be occurring at pressures greater than 150 MPa. Tensile strength decreases if compression pressure and solid faction are too high. FIG. 4 provides compression profiles of Compound 1A API.

Compound 1A had significant sticking issues with the punch tips during compression, ejection forces were also very high. The die wall was manually lubricated with magnesium stearate to mitigate this issue.

### Compound 1A Tablet Development

### Excipient Compatibility

Excipient compatibility of Compound 1A was evaluated by manufacturing binary blends with a variety of commonly used excipients (filler, binders, lubricants, etc.). Samples were tested for assay/impurity, appearance, and XRPD at 4 and 8 week timepoints. Storage conditions were 40° C. and 75% RH open and closed. Compound 1A showed similar impurity growth (˜0.75%) in open and closed conditions between 4 and 8 weeks. After 8 weeks, CAB-O-SIL® showed most impurity growth compared to other excipients. Magnesium stearate and lactose also had elevated growth.

Table 10 shows impurity results below.

TABLE 10

| | Total Impurity (%) | | | | |
|---|---|---|---|---|---|
| | | 4 weeks/40° | 4 weeks/40° | 8 weeks/40° | 8 weeks/40° |
| | | C./75% RH/ | C./75% RH/ | C./75% RH/ | C./75% RH/ |
| Sample Description | t = 0 | OPEN | CLOSED | OPEN | CLOSED |
| 1:9 (w:w) Compound | 0.67 | 1.08 | 1.36 | 1.63 | 0.98 |
| 1A:AVICEL ® PH-102 | | | | | |
| 1:9 (w:w) Compound | 0.93 | 1.48 | 1.40 | 1.80 | 1.67 |
| 1A:PARTECK ® M100 | | | | | |
| 1:9 (w:w) Compound | 0.81 | 1.49 | 2.57 | 1.15 | 2.42 |
| 1A:Fast Flo 316 | | | | | |
| 1:4 (w:w) Compound | 0.77 | 1.18 | 1.62 | 1.13 | 1.18 |
| 1A:Kollidon CL | | | | | |
| 1:4 (w:w) Compound | 0.65 | 1.28 | 1.58 | 1.32 | 1.12 |
| 1A:AC-DI-SOL ® | | | | | |
| 1:4 (w:w) Compound | 0.71 | 1.39 | 1.23 | 2.10 | 1.47 |
| 1A:CAB-O-SIL ® | | | | | |
| 1:4 (w:w) Compound 1A:Talc | 0.66 | 1.23 | 1.49 | 1.31 | 1.26 |
| 1:4 (w:w) Compound | 0.65 | 1.21 | 1.33 | 0.99 | 1.42 |
| 1A:Nisso HPC SSL-SFP | | | | | |
| 1:4 (w:w) Compound | 0.69 | 1.26 | 1.45 | 1.00 | 1.20 |
| 1A:Kollidon K30 (povidone) | | | | | |
| 1:4 (w:w) Compound | 0.74 | 1.12 | 1.69 | 0.92 | 1.58 |
| 1A:Kolliphor SLS fine | | | | | |
| 1:4 (w:w) Compound | 0.65 | 1.67 | 2.18 | 2.09 | 1.27 |
| 1A:Mg. Stearate | | | | | |
| 1:4 (w:w) Compound | 0.53 | 0.90 | 0.97 | 0.75 | 1.04 |
| 1A:PRUV ® SSF | | | | | |
| Compound 1A API control | 0.75 | 1.48 | 1.52 | 1.45 | 1.31 |

Some samples showed appearance change after 8 weeks in the open condition, but no changes were observed in any closed condition. Most open/closed condition samples showed similar diffractogram comparted with t=0. After aging 4 weeks, two diffraction peaks (5.50 and 8.30) disappeared in 1:4 Compound 1A:Kolliphor SLS sample compared to t=0, likely due to moisture uptake.

### Manufacture of Prototype Compound 1A Tablets

Based on excipient compatibility data, three formulations were nominated to evaluate feasibility of min/max target doses (100 and 250 mg Compound A) while keeping overall tablet weight fixed at 555 mg. Tablet formulations are summarized in Table 11. The use of Talc was investigated as an alternative to CAB-O-SIL® due to the potential compatibility issue observed.

TABLE 11
Compound 1A prototype tablet formulation
where drug loading was ranged from 20-50%

| | | Compound | Compound | Compound |
|---|---|---|---|---|
| | | 1A | 1A | 1A |
| Compound 1A | Drug Carrier | 50.00 | 20.00 | 50.00 |
| API (90% Assay) | | | | |
| AVICEL ® PH 102 | Filler/Diluent | 23.00 | 38.00 | 21.75 |
| (Microcrystalline | | | | |
| Cellulose) | | | | |
| PARTECK ® | Filler/Diluent | 23.00 | 38.00 | 21.75 |
| M100 (Mannitol) | | | | |
| AC-DI-SOL ® | Disintegrant | 3.00 | 3.00 | 3.00 |
| (Croscarmellose | | | | |
| Sodium) | | | | |
| CAB-O-SIL ® | Glidant | 0.50 | 0.50 | — |
| (Fumed Silica) | | | | |
| Talc | Glidant | — | — | 3.00 |
| Sodium Stearyl | Lubricant | 0.50 | 0.50 | 0.50 |
| Fumarate (PRUV ®) | | | | |

| | | | |
|---|---|---|---|
| Total: | 100.00 | 100.00 | 100.00 |
| Total Tablet Weight (mg): | 555.00 | 555.00 | 555.00 |
| Dose (mg Compound 1A): | 250 | 100 | 250 |

All intragranular materials were blended in a turbula blender, passed through a 25 mesh (700 micron) screen, and turbula blended for an additional 5 minutes. Blend was then granulated via slug & mill using 0.875″ flat faced tooling and targeting a solid fraction of 0.55-0.65. The slugs were then milled using a hand mill with 1000 μm mesh screen. Based on yield, corrected amounts of extragranular materials were added to the granulation (SSF was de-lumped with 20 mesh prior to addition) and turbula blended for 5 minutes. Total batch size was approximately 25 g per formulation.

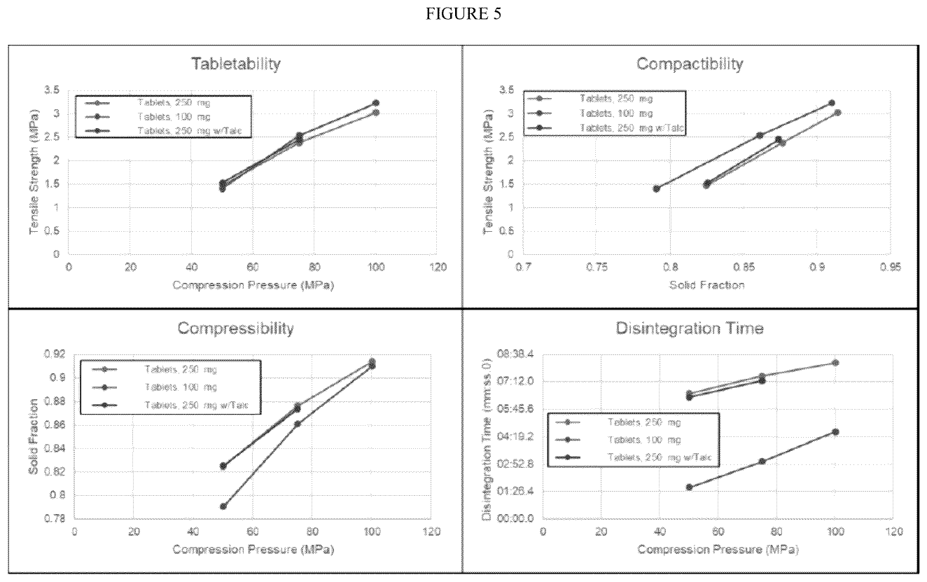

Prior to slug and mill, the intra-granular blends were evaluated for direct compression feasibility by observing how easily blend would flow into the tooling die. All blends appeared to flow into the die well, but were not able to reach the target tablet weight at the maximum die depth. The API bulk density is too low for these formulations to work via direct compression. Prototype tablets were manufactured on a manual press. Tabletability, compressibility, compactability, and disintegration profiles were generated for all formulations using 0.3010″×0.6200″ modified oval tooling over the compression range 50-100 MPa (FIG. 5). All formulations had similar compression characteristics and were able to form strong tablets at low compression pressures. Formulations containing 50% Compound 1A had longer disintegration times than the 20% Compound 1 containing formulation. Tablets for stability were manufactured at 50 MPa and 75 MPa for the 250 and 100 mg tablets, respectively.

Severe picking was observed during compression of stability supplies, to the point where the entire cap of the tablet was removed due to sticking to the tooling.

Additional 2.0% PRUV® (sodium steryl fumarate lubricant) was added to all formulations for the remainder of the stability tablets. This adjustment resolved the sticking issue at the prototype scale. The adjusted formulations can be seen below (Table 12). Fill weight was held constant at 555 mg, which reduced the resulting potency of the tablets by 1.5%.

TABLE 12
Adjusted Compound 1A tablet formulations after the additional 2.0% PRUV ® was added

| | | Compound 1A | Compound 1A | Compound 1A |
|---|---|---|---|---|
| | | Tablets, 250 mg | Tablets, 100 mg | Tablets, 250 mg |
| Component | Function | (wt. %) | (wt. %) | (w/Talc) (wt. %) |
| Compound 1A API | Drug Carrier | 49.25 | 19.70 | 49.25 |
| (90% Assay) | | | | |
| AVICEL ® PH 102 | Filler/Diluent | 22.66 | 37.43 | 21.42 |
| (Microcrystalline | | | | |
| Cellulose) | | | | |
| PARTECK ® M100 | Filler/Diluent | 22.66 | 37.43 | 21.42 |
| (Mannitol) | | | | |
| AC-DI-SOL ® | Disintegrant | 2.96 | 2.96 | 2.96 |
| (Croscarmellose | | | | |
| Sodium) | | | | |
| CAB-O-SIL ® | Glidant | 0.49 | 0.49 | — |
| (Fumed Silica) | | | | |
| Talc | Glidant | — | — | 2.96 |
| Sodium Stearyl | Lubricant | 2.00 | 2.00 | 2.00 |
| Fumarate (PRUV ®) | | | | |

| | | | |
|---|---|---|---|
| Total: | 100.00 | 100.00 | 100.00 |
| Total Tablet Weight (mg): | 555.00 | 555.00 | 555.00 |
| Dose (mg Compound 1A): | 246 | 99 | 246 |

### Analytical Characterization of Immediate Release Stability Tablets

The three PK tablet formulations were characterized by assay and impurities by HPLC, appearance, water content by KF, and biorelevant non-sink dissolution. Water content values were typical for tablets and were consistent across both tablet strengths.

TABLE 13
Water Content and Appearance Data for Compound 1A PK Tablets

| | | Water |
|---|---|---|
| | | Content |
| Formulation | Appearance | (wt %) |
| Tablets, 250 mg | Off-White Tablet | 3.14 |
| Tablets, 100 mg | Off-White Tablet | 2.96 |
| Tablets w/Talc, 250 mg | Off-White Tablet | 2.24 |

The dissolution performance of the PK tablets was tested in a biorelevant non-sink dissolution experiment at two different dosing concentrations. The first dosing concentration was 4 mgA/mL gastric->2 mgA/mL intestinal (Table 14), and the second was 2 mgA/mL gastric->1 mgA/mL intestinal (Table 15). At a 4 mgA/mL dosing concentration, all tablets exhibited a similar dissolution performance regardless of tablet strength with a similar trend observed at the 2 mgA/mL dosing concentration. Both 250 mgA tablets performed nearly identically to one another at both dosing concentrations with Talc having no noticeable impact on dissolution performance. Increasing the dosing concentration to 4 mgA/mL saw no significant improvement over the 2 mgA/mL dosing concentration thus a dosing concentration of 2 mgA/mL was determined to be sufficient for future dissolution work.

TABLE 14
Non-Sink Dissolution Data for Compound 1A PK Tablets at
4 mgA/L −> 2 mgA/mL dosing concentration

| | Cₘₐₓ | | AUC₃₅₋₂₁₀ |
|---|---|---|---|
| | FaSSIF | C₂₁₀ | FaSSIF |
| Formulation | (μgA/mL) | (μgA/mL) | (min*μgA/mL) |
| Tablets, 250 mg | 1043 | 1043 | 156900 |
| Tablets, 100 mg | 968 | 968 | 163800 |
| Tablets w/Talc, 250 mg | 896 | 896 | 150200 |

TABLE 15
Non-Sink Dissolution Data for Compound 1A PK Tablets at
2 mgA/L −> 1 mgA/mL dosing concentration

| | Cₘₐₓ | | AUC₃₅₋₂₁₀ |
|---|---|---|---|
| | FaSSIF | C₂₁₀ | FaSSIF |
| Formulation | (μgA/mL) | (μgA/mL) | (min*μgA/mL) |
| Tablets, 250 mg | 834 | 833 | 145600 |
| Tablets, 100 mg | 827 | 824 | 144100 |
| Tablets w/Talc, 250 mg | 822 | 822 | 139700 |

Assay/Impurities analysis of the PK tablets by HPLC shows similar total relative substances compared with the bulk API (Table 16), indicating that no chemical degradation occurred during the tablet manufacturing processes. Assay values were observed to be slightly low, but close to the intended label claim. The 100 mgA tablets were made with sodium stearyl fumarate (SSF) as an excipient. SSF has the possibility of showing up in the chromatograms so an SSF sample was injected to identify any peaks related to this excipient.

TABLE 16
Assay/Impurities of Compound 1A
PK Tablets Compared with Bulk API

| | | Compound | Compound | Compound |
|---|---|---|---|---|
| | | 1A | 1A | 1A |
| | Compound | Tablets, | Tablets, | Tablets with |
| RRT | 1A | 250 mg | 100 mg | Talc, 250 mg |
| 0.82 | | <0.05% | <0.05% | |
| 0.83 | | <0.05% | | |
| 0.98 | 0.06% | 0.06% | 0.06% | 0.06% |
| 1.14 | 0.07% | 0.05% | 0.05% | 0.06% |
| 1.15 | 0.10% | 0.07% | 0.07% | 0.07% |
| 1.18 | 0.12% | 0.09% | 0.09% | 0.08% |
| 1.23 | | <0.05% | <0.05% | <0.05% |
| 1.26 | 0.05% | 0.06% | 0.06% | 0.06% |
| 1.28 | <0.05% | <0.05% | <0.05% | <0.05% |
| 1.32 | 0.26% | 0.28% | 0.28% | 0.28% |
| 1.55 | <0.05% | <0.05% | <0.05% | <0.05% |
| Total | 0.66% | 0.60% | 0.61% | 0.61% |
| Impurity | | | | |
| Potency | — | 237.3 ± 0.2 | 96.1 ± 0.5 | 237.6 ± 0.0 |
| | | mg | mg | mg |

All Compound 1A tablet formulations were placed on stability to assess the physical appearance and chemical stability. Tablets were held placed in HDPE bottles with 0.5 g silica gel desiccant and heat-induction sealed. Samples were stored at 40° C./75% RH for the duration of this study.

Stability samples were pulled and analyzed after 1 month and 3 months. Performed tests included appearance, water content, assay and related substances, and biorelevant non-sink dissolution. No change in appearance was observed in any tablet formulation at either pull time point. Minimal change in water content was observed over the three-month study. Most tablet formulations had a water content between 2.5-3.0 (Table 17). These values are typical for tablet formulations.

TABLE 17
Water Content and Appearance Data for PK Tablets

| | | | Water |
|---|---|---|---|
| | Storage | | Content |
| Formulation | Condition | Appearance | (wt %) |
| Compound 1A Tablets, | t = 0 | Off-White | 3.14 |
| 250 mg | | Tablet | |
| | 1 month/ | Off-White | 2.96 |
| | 40° C./75% | Tablet | |
| | RH/CLOSED | | |
| | 3 month/ | Off-White | 2.96 |
| | 40° C./75% | Tablet | |
| | RH/CLOSED | | |
| Compound 1A Tablets, | t = 0 | Off-White | 2.96 |
| 100 mg | | Tablet | |
| | 1 month/ | Off-White | 2.78 |
| | 40° C./75% | Tablet | |
| | RH/CLOSED | | |
| | 3 month/ | Off-White | 2.53 |
| | 40° C./75% | Tablet | |
| | RH/CLOSED | | |
| Compound 1A Tablets | t = 0 | Off-White | 2.24 |
| w/Talc, 250 mg | | Tablet | |
| | 1 month/ | Off-White | 2.91 |
| | 40° C./75% | Tablet | |
| | RH/CLOSED | | |
| | 3 month/ | Off-White | 2.67 |
| | 40° C./75% | Tablet | |
| | RH/CLOSED | | |

The dissolution performance of the stability tablets was tested in a biorelevant non-sink dissolution experiment at a dosing concentration of 2 mgA/mL gastric->1 mgA/mL intestinal.

Results from the dissolution testing (Table 18) indicated that all tablet formulations had comparable dissolution performance to their respective t=0 dissolution performance at both the one month and the three-month stability pulls.

TABLE 18
Non-Sink Dissolution Data for Compound 1A PK Tablet Stability
Samples at 2 mgA/L −> 1 mgA/mL dosing concentration

| | Cₘₐₓ | | AUC₃₅₋₂₁₀ |
|---|---|---|---|
| | FaSSIF | C₂₁₀ | FaSSIF |
| Formulation | (μgA/mL) | (μgA/mL) | (min*μgA/mL) |
| Compound 1A 250 mg, | 880 | 876 | 152800 |
| 3M/40° C./75% RH/CLOSED | | | |
| Compound 1A Tablets w/Talc, | 869 | 869 | 150700 |
| 250 mg, | | | |
| 3M/40° C./75% RH/CLOSED | | | |
| Compound 1A Tablets, 100 mg, | 881 | 881 | 148600 |
| 3M/40° C./75% RH/CLOSED | | | |
| Compound 1A Tablets, 250 mg, | 880 | 880 | 153100 |
| 1M/40° C./75% RH/CLOSED | | | |
| Compound 1A Tablets w/Talc, | 862 | 862 | 149800 |
| 250 mg, | | | |
| 1M/40° C./75% RH/CLOSED | | | |
| Compound 1A Tablets, 100 mg, | 837 | 837 | 142900 |
| 1M/40° C./75% RH/CLOSED | | | |
| Compound 1A Tablets, 250 mg, | 834 | 833 | 147300 |
| t = 0 (rerun) | | | |
| Compound 1A Tablets w/Talc, | 827 | 824 | 145800 |
| 250 mg, t = 0 (rerun) | | | |
| Compound 1A Tablets, 100 mg, | 822 | 822 | 141300 |
| t = 0 (rerun) | | | |

Assay/Impurities analysis of the stability tablets by HPLC shows significant impurity growth after aging for one month and continued growth through the three month time point compared when compared to the bulk API (Table 19, Table 20, Table 21). The majority of this impurity growth occurred at RRTs of ˜1.30, 1.68 and 1.82 for all tested tablets. A shift in the Compound 1A peak elution time was also observed in the three-month stability samples resulting in some RRT variation among the impurity peaks particularly with the impurity observed at RRT 1.32 shifting to 1.34 in the 1-month samples to 1.30 in the 3 month samples. In addition to the RRT variation, peaks were observed at RRT 0.90 and 1.14. These peaks were also observed in the diluent and thus not included in the total impurities count. After three months of aging, the 100 mg tablets had the greatest impurity growth.

TABLE 19
Assay/Impurities of 250 mg Compound 1A PK Tablet
Stability Samples Compared with Bulk API

| RRT | | | Compound 1A Tablets, 250 mg | |
|---|---|---|---|---|
| Condition | Compound 1A | | 1M/40° C./75% | 3M/40° C./75% |
| RRT | RT | t = 0 | RH CLOSED | RH CLOSED |
| 0.82 | | <0.05% | | |
| 0.83 | | <0.05% | | |
| 0.96 | | | <0.05% | |
| 0.98 | 0.06% | 0.06% | 0.07% | 0.07% |
| 1.06 | | | | 0.11% |
| 1.08 | | | | 0.09% |
| 1.14 | 0.07% | 0.05% | 0.15% | |
| 1.15 | 0.10% | 0.07% | 0.07% | |
| 1.18 | 0.12% | 0.09% | 0.09% | |
| 1.23 | | <0.05% | | |
| 1.26 | 0.05% | 0.06% | 0.10% | 0.09% |
| 1.27 | <0.05% | <0.05% | 0.08% | 0.07% |
| 1.29 | | | | <0.05% |
| 1.30 | | | | 1.16% |
| 1.32 | 0.26% | 0.28% | | |
| 1.34 | | | 0.73% | |
| 1.45 | | <0.05% | <0.05% | |
| 1.53 | | | | 0.12% |
| 1.55 | <0.05% | | | |
| 1.57 | | | 0.09% | |
| 1.62 | | | | <0.05% |
| 1.68 | | | | 0.06% |
| 1.82 | | | <0.05% | 0.10% |
| Total | 0.66% | 0.60% | 1.36% | 1.87% |
| Potency | 88.20% | 237.3 ± 0.2 | 239.4 ± 0.2 | 239.7 ± 1.1 |
| | | mg | mg | mg |

TABLE 20
Assay/Impurities of 100 mg Compound 1A PK Tablet
Stability Samples Compared with Bulk API

| RRT | | | Compound 1A Tablets, 100 mg | |
|---|---|---|---|---|
| Condition | Compound 1A | | 1M/40° C./75% | 3M/40° C./75% |
| RRT | RT | t = 0 | RH CLOSED | RH CLOSED |
| 0.82 | | <0.05% | | |
| 0.96 | | | <0.05% | |
| 0.98 | 0.06% | 0.06% | 0.07% | 0.07% |
| 1.06 | | | | 0.10% |
| 1.08 | | | | 0.09% |
| 1.14 | 0.07% | 0.05% | 0.14% | |
| 1.15 | 0.10% | 0.07% | 0.08% | |
| 1.18 | 0.12% | 0.09% | 0.09% | <0.05% |
| 1.23 | | <0.05% | | |
| 1.26 | 0.05% | 0.06% | 0.08% | 0.09% |
| 1.27 | <0.05% | <0.05% | 0.07% | 0.09% |
| 1.29 | | | | <0.05% |
| 1.30 | | | | 1.07% |
| 1.32 | 0.26% | 0.28% | | |
| 1.34 | | | 0.64% | |
| 1.45 | | | <0.05% | |
| 1.53 | | | | 0.10% |
| 1.55 | <0.05% | <0.05% | | |
| 1.57 | | | 0.07% | |
| 1.62 | | | | <0.05% |
| 1.68 | | | <0.05% | 0.07% |
| 1.72 | | | <0.05% | |
| 1.82 | | | <0.05% | 0.25% |
| Total | 0.66% | 0.61% | 1.24% | 1.91% |
| Potency | 88.20% | 96.1 ± 0.5 | 97.0 ± 0.3 | 98.9 ± 0.7 |
| | | mg | mg | mg |

TABLE 21
Assay/Impurities of 250 mg Compound 1A PK Tablet
w/Talc Stability Samples Compared with Bulk API

| | | | Compound 1A Tablets with | |
|---|---|---|---|---|
| RRT | | | Talc, 250 mg | |
| Condition | Compound 1A | | 1M/40° C./75% | 3M/40° C./75% |
| RRT | RT | t = 0 | RH CLOSED | RH CLOSED |
| 0.96 | | | <0.05% | |
| 0.98 | 0.06% | 0.06% | 0.07% | 0.07% |
| 1.06 | | | | 0.09% |
| 1.08 | | | | 0.08% |
| 1.14 | 0.07% | 0.06% | 0.16% | |
| 1.15 | 0.10% | 0.07% | 0.07% | |
| 1.18 | 0.12% | 0.08% | 0.08% | <0.05% |
| 1.23 | | <0.05% | | |
| 1.26 | 0.05% | 0.06% | 0.09% | 0.09% |
| 1.27 | <0.05% | <0.05% | 0.07% | 0.08% |
| 1.29 | | | | <0.05% |
| 1.30 | | | | 1.07% |
| 1.32 | 0.26% | 0.28% | | |
| 1.34 | | | 0.65% | |
| 1.53 | | | | 0.13% |
| 1.55 | <0.05% | <0.05% | | |
| 1.57 | | | 0.08% | |
| 1.62 | | | | <0.05% |
| 1.68 | | | | 0.06% |
| 1.82 | | | <0.05% | 0.10% |
| Total | 0.66% | 0.61% | 1.26% | 1.77% |
| Potency | 88.20% | 237.6 ± 0.0 | 238.9 ± 0.5 | 240.4 ± 0.1 |
| | | mg | mg | mg |

### Scale Up and Demonstration Batch Manufacture of Immediate Release Tablets

### Compound 1A Scale Up Development (GMP API)

The tablet formulation containing 50% Compound 1A API and CAB-O-SIL® was nominated after prototype stability results. Minor formulations changes were implemented:
Increased PRUV® from 0.5 to 2 to mitigate punch sticking
Shifted components extra-granular to ensure compressibility after roller compaction
Increased AC-DI-SOL® from 3 to 6 to improve disintegration

The formulation was to be used as a common granulation to accommodate 250 and 100 mg tablet strengths (Table 22).

TABLE 22
Nominated scale up formulation for 250 and 100 mg tablets.

| | | | 100 mg | 250 mg |
|---|---|---|---|---|
| Component | Function | Weight % | (mg/tab) | (mg/tab) |
| Compound 1A API, 90% Assay | Drug Carrier | 50.00 | 111.00 | 277.50 |
| AVICEL ® PH 105 | Filler/Diluent | 17.25 | 38.30 | 95.74 |
| PARTECK ® M100 | Filler/Diluent | 17.25 | 38.30 | 95.74 |
| AC-DI-SOL ® | Disintegrant | 3.00 | 6.66 | 16.65 |
| CAB-O-SIL ® | Glidant | 0.50 | 1.11 | 2.78 |
| Sodium Stearyl Fumarate | Lubricant | 1.00 | 2.22 | 5.55 |
| Intra-Granular Total: | | 89.00 | 197.58 | 493.95 |
| AVICEL ® PH-200 | Compression Aid | 7.00 | 15.54 | 38.85 |
| AC-DI-SOL ® | Disintegrant | 3.00 | 6.66 | 16.65 |
| Sodium Stearyl Fumarate | Lubricant | 1.00 | 2.22 | 5.55 |

| | | | |
|---|---|---|---|
| Extra-Granular Total: | 11.00 | 24.42 | 61.05 |
| Total: | 100.00 | 222.00 | 555.00 |

Extreme flow issues were observed during the roller compaction process. The intra-granular blend appeared to be adhering to the screw and bowl of the roller compactor to the point where no material was being passed through. A wide range of roller compaction parameters and starve feeding were attempted, but nothing seemed to help. The intra-granular blend was then diluted to 25% Compound 1A using 1:1 MCC:Mannitol, but no improvement was observed. The cause of this is likely the properties of the API, which appeared to have an affinity for stainless steel. The scale up batch was halted where reformulation was determined to be necessary.

### Tablet Reformulation Trials

Reformulation efforts were designed to investigate the impact of small vs. large particle size excipients and their ability to process through the micro roller compactor. If unsuccessful, the blends were to be diluted down to 37.5% Compound 1A, then 25% if necessary. The overall batch size for the blends was 75 g, formulation table can be seen below (Table 23).

TABLE 23
Small and large PSD formulations to be evaluated
for processability on the micro roller compactor

| | Small PSD | Large PSD |
|---|---|---|
| | Excipients | Excipients |
| Component | (wt. %) | (wt. %) |
| Compound 1A (API), 92.7% | 50.00 | 50.00 |
| Assay | | |
| AVICEL ® PH 105 (25 μm) | 17.25 | — |
| AVICEL ® PH-200 (200 μm) | — | 17.25 |
| PEARLITOL ®25 C (25 μm) | 17.25 | — |
| PARTECK ® M200 (200 μm) | — | 17.25 |
| AC-DI-SOL ® | 3.00 | 3.00 |
| CAB-O-SIL ® | 0.50 | 0.50 |
| PRUV ® | 1.00 | 1.00 |
| Intra-Granular Total: | 89.00 | 89.00 |
| AVICEL ® PH-200 | 7.00 | 7.00 |
| Ac-Di-Sol | 3.00 | 3.00 |
| PRUV ® | 1.00 | 1.00 |
| Extra-Granular Total: | 11.00 | 11.00 |
| Total | 100.00 | 100.00 |

When the reformulated blends were added to the roller compactor, they appeared to process with no issue and no hang up on the screw or bowl was observed. This result was very unexpected and when bulk/tapped densities were compared, both new formulations had significantly better flow based on Hausner ratio and visual comparison (Table 24).

TABLE 24
Bulk and tapped density values of failed Demonstration batch
and reformulated (small vs. large PSD) intra-granular blends

| | Bulk Density | Tapped Density | |
|---|---|---|---|
| Batch | (g/cc) | (g/cc) | Hausner Ratio |
| Demo (failed) | 0.11 | 0.23 | 2.09 |
| Small PSD | 0.17 | 0.31 | 1.82 |
| Large PSD | 0.18 | 0.30 | 1.67 |

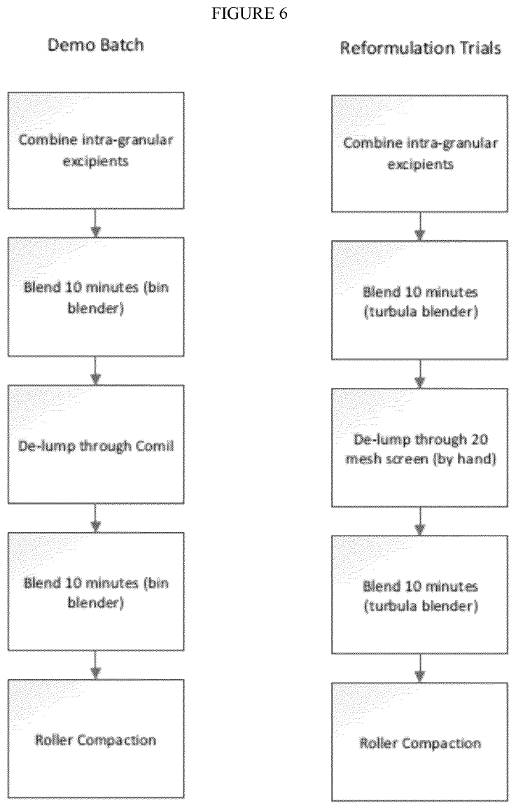

It was expected that the failed Demonstration batch would have a bulk density value between that of the large and small PSD blends. When looking at the process, because the reformulated blends were much smaller than the scale up batch, they were de-lumped through a 25 mesh screen by hand instead of being processed through the U5 Comil (FIG. 6). This de-lumping step was the major difference in blend prep. It was hypothesized that de-lumping through the Comil was the root cause of the flow issues.

With this finding, the 50% Compound 1A tablet formulation was determined to be feasible and an additional demonstration batch was to be performed.

### Compound 1A Demonstration Batch, Updated Process

Based on reformulation observations and favorable flow properties, the large PSD excipient formulation was nominated for the demonstration batch (Table 25). Overall batch size was 500 g.

TABLE 25
Demonstration batch tablet formulation utilizing
large particle size excipients

| | Large PSD | |
|---|---|---|
| | Excipients | |
| Component | (wt. %) | 250 mg (mg/tab) |
| Compound 1A (API), 92.7% | 50.00 | 270.00 |
| Assay | | |
| AVICEL ® PH-200 (200 μm) | 17.25 | 93.15 |
| PARTECK ® M200 (200 μm) | 17.25 | 93.15 |
| AC-DI-SOL ® | 3.00 | 16.20 |
| CAB-O-SIL ® | 0.50 | 2.70 |
| PRUV ® | 1.00 | 5.40 |
| Intra-Granular Total: | 89.00 | 480.60 |
| AVICEL ® PH-200 | 7.00 | 37.80 |
| Ac-Di-Sol | 3.00 | 16.20 |
| PRUV ® | 1.00 | 5.40 |
| Extra-Granular Total: | 11.00 | 59.40 |
| Total | 100.00 | 540.00 |

In the process for manufacture of the demonstration batch, the intra-granular de-lumping step in the previous batch was omitted in order to mitigate adverse API flow issues.

During roller compaction a full ribbon was not observed and there was difficulty obtaining a brittle ribbon, even at aggressive conditions. Throughput at nominated conditions was very slow (˜0.25 kg/hr) and flow issues appeared to still be a factor, but it was able to process.

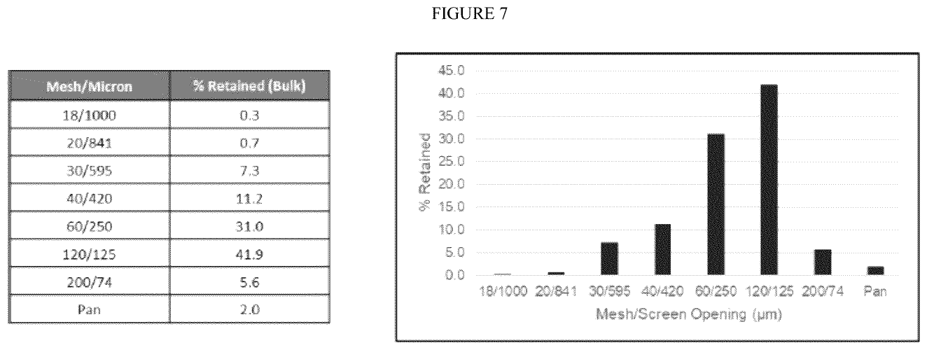

The soft ribbons resulted in granulation with a high level of granules around 120 μm particle size and very little coarse material. FIG. 7 provides particle size distribution of Compound 1 A demonstration batch granules where a high level of fine material was observed due to the low ribbon solid fraction.

Final blend did appear to have acceptable flow properties by Hausner ratio and by visual observation. Blend characterization of intra-granular blend, granulation, final blend can be seen in Table 26.

TABLE 26
Bulk, tapped, and true density values of in
process demonstration batch tablet blend

| | Bulk Density | Tapped Density | Hausner | True Density |
|---|---|---|---|---|
| Blend Step | (g/cc) | (g/cc) | Ratio | (g/cc) |
| Intra-Granular | 0.26 | 0.38 | 1.46 | 1.53 |
| Granulation | 0.34 | 0.52 | 1.53 | 1.53 |
| Final Blend | 0.41 | 0.55 | 1.34 | — |

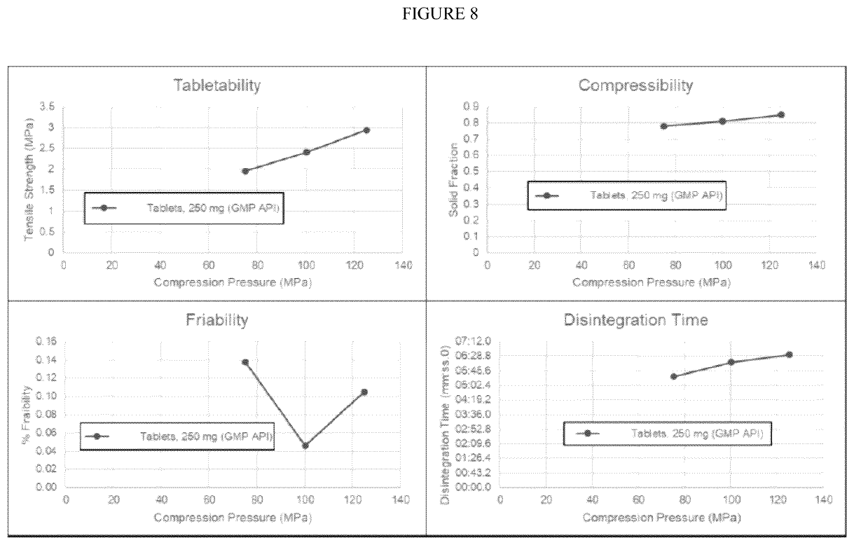

Final blend was very compressible and tablets of high tensile strength were achieved at low compression pressures. Disintegration time ranged from 5-7 minutes and friability was well below the 1% weight loss threshold and no defects on fried tablets were observed. Compression profiles can be seen in FIG. 8.

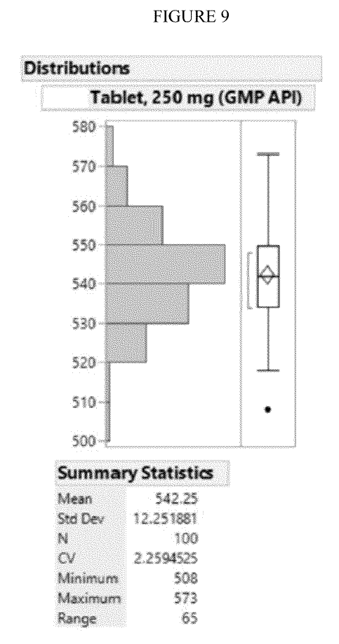

Bulk tablets were compressed at 100 MPa with an acceptable range from 75-125 MPa. Composite sample of 100 tablets were analyzed for weight distribution where mean tablet weight was 542 mg and % RSD was 2.26 (5 tablets found outside of ±5% of target). On paper the weight distribution looks acceptable, however, flow issues were observed throughout the run. Rat-holing in the tablet hopper occurred multiple times and constant weight adjustments were required. The tablet weight distribution can be seen in FIG. 9.

Tablet appearance during bulk tablet compression changed from glossy, smooth tablets to dull and rough surface tablets. The rough tablet surface was caused by material accumulating on the punch tips during compression. The significant amount of buildup is likely caused by properties of Compound 1A, which was also why weight control during processing was challenging.

The physical characteristics of the tablet were acceptable, but final blend had poor flow through the tablet press and caused material accumulation on the punch tips, which will likely get worse at larger scale. Overall, this formulation was determined to not be suitable for GMP manufacture due to the Korsch XL100 used in GMP being more sensitive to poorly flowing powders, than the Piccola used for the demonstration batch.

### Punch Sticking Evaluation

Major reformulation was determined to be necessary to troubleshoot the punch sticking and flow issues observed during the demonstration batch. Due to low availability of API, several changes were implemented at one time. Nominated formulation can be seen in Table 27, summary of changes included:
Dilute Compound 1A from 50% down to 27% (1000 mg total tablet weight for 250 mg Compound 1A dose)
Utilize medium particle size MCC and mannitol grades (˜100 μm)
Increase extra-granular PRUV® from 1 to 2%

TABLE 27
Nominated tablet formulation for punch sticking evaluation.

| Component | Weight % | mg/Tablet |
|---|---|---|
| Compound 1A (API), | 27.00 | 270.00 |
| 92.7% Assay | | |
| AVICEL ® PH-102 | 28.25 | 287.50 |
| PARTECK ® M100 | 28.25 | 287.50 |
| AC-DI-SOL ® | 3.00 | 30.00 |
| CAB-O-SIL ® | 0.50 | 5.00 |
| PRUV ® | 1.00 | 10.00 |
| Intra-Granular Total: | 88.00 | 880.00 |
| AVICEL ® PH-200 | 7.00 | 70.00 |
| Ac-Di-Sol | 3.00 | 30.00 |
| PRUV ® | 2.00 | 10.00 |
| Extra-Granular Total: | 12.00 | 120.00 |
| Total | 100.00 | 1000.00 |

Overall blend size was 37 g and granulation was prepared via slug and mill similar to the prototype tablet batches, except tablets were compressed using the compaction simulator.

Tablets were compressed using 0.25″ SRC tooling, 150 MPa compression pressure, and 100 mg total weight in order to see accumulation after 100 and 200 compression events. Tablet tooling after compression showed minor accumulation/hazing after 100 tablets, and similar amount after 200. The accumulation did not appear to worsen between 100-200 compressions. Material that did stick to the punch tips was easily cleaned off with a dry wipe.

Tablets appearance remained the same from tablet #1-#200, white to off-white with a smooth glossy finish. Tablet breaking force was 7 kP, ˜2.45 MPa tensile strength showing blend has high compressibility.

New tooling, 0.3750″×0.7480″ at 150 MPa compression pressure was nominated to accommodate the reformulated 250 mg Tablet (1000 mg total weight). This tooling appeared to be appropriate for this formulation and was nominated for the subsequent demo batch.

### Tablet Demonstration, Scale Up

The formulation from the punch sticking study was determined to be successful and was nominated for the demonstration batch (Table 28), overall batch size was 1000 g.

TABLE 28
Nominated formulation for 250 mg tablet demonstration batch

| Component | Weight % | mg/Tablet |
|---|---|---|
| Compound 1 A (API), | 27.00 | 270.00 |
| 92.7% Assay | | |
| AVICEL ® PH-102 | 28.25 | 287.50 |
| PARTECK ® M100 | 28.25 | 287.50 |
| AC-DI-SOL ® | 3.00 | 30.00 |
| CAB-O-SIL ® | 0.50 | 5.00 |
| PRUV ® | 1.00 | 10.00 |
| Intra-Granular Total: | 88.00 | 880.00 |
| AVICEL ® PH-200 | 7.00 | 70.00 |
| AC-DI-SOL ® | 3.00 | 30.00 |
| PRUV ® | 2.00 | 10.00 |
| Extra-Granular Total: | 12.00 | 120.00 |
| Total | 100.00 | 1000.00 |

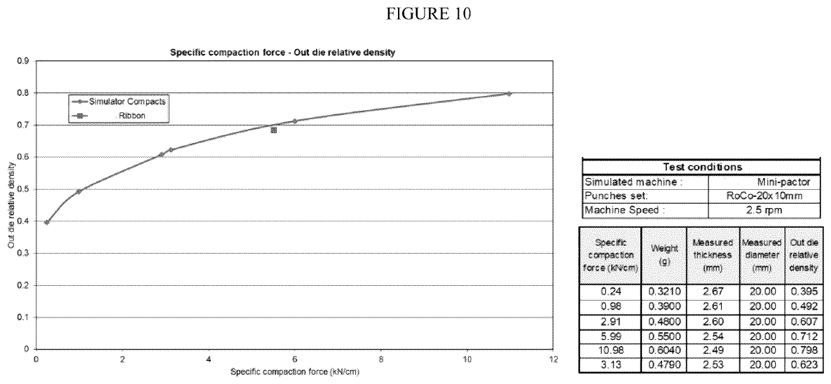

The intra-granular blend was compressed using a compaction simulator using a simulated roll speed of 2.5 rpm and fixed gap setting of 2.5 mm. Press forced was increased from 0.24-10.98 kN/cm and the simulated ribbon solid fraction was recorded (out die relative density). Solid fractions ranged from 0.395-0.798. Target solid fraction of 0.65-0.70 was nominated based on the feel and brittleness of the compacts. Using the graph in FIG. 10, a press force of ˜5.5 kN/cm was nominated to achieve the target solid fraction.

The nominated roller compacter parameters were used for start up of the roller compaction process. Ribbon sample was obtained once the roller compacter reached a steady state. The resulting ribbon solid fraction was 0.68, which was within the target window. Intra-blend was then exhausted at the nominated settings. Intra-granular blend processed through the roller compacter without issue. Total roller compaction time was ˜10 minutes and throughput was estimated to be 7-10 kg/hr.

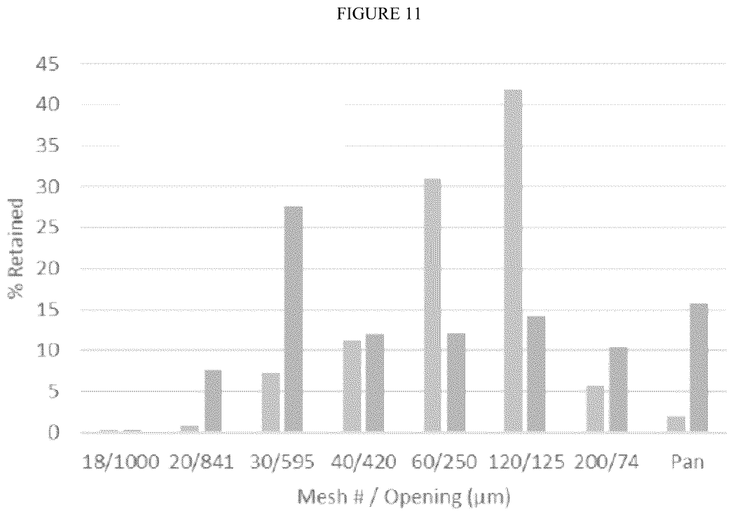

Granulation particle size distribution (sieve analysis) data was compared to the previous demonstration batch that has flow issues through the tablet press. The granules from the nominated roller compacter has a much higher level of coarse material due to the increased ribbon solid fraction (FIG. 11).

The roller compacter granulation had higher level of coarse material. Bulk, tapped, true densities were also characterized for the intra, granulation, and final blend. Results can be seen in Table 29.

TABLE 29
Bulk, tapped, and true density values
of in process Compound 1A blend samples

| | Bulk | Tapped | | True |
|---|---|---|---|---|
| | Density | Density | Hausner | Density |
| Blend Step | (g/cc) | (g/cc) | Ratio | (g/cc) |
| Intra-Granular | 0.34 | 0.52 | 1.53 | 1.52 |
| Granulation | 0.52 | 0.72 | 1.38 | 1.52 |
| Final Blend | 0.53 | 0.75 | 1.42 | 1.51 |

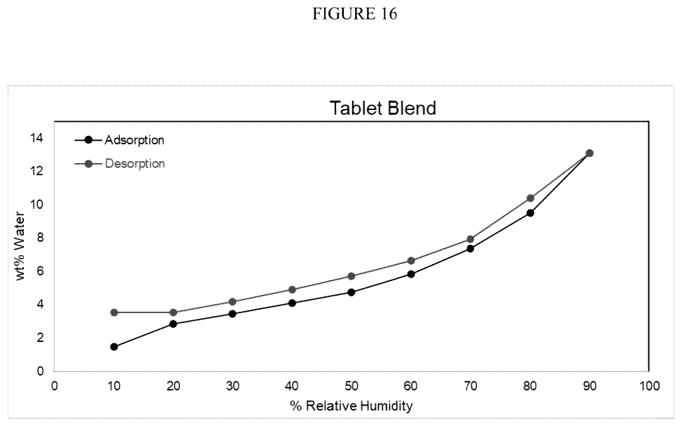

Tablet compression profiles were generated by compression from 100-150 MPa pressure where final blend was found to be highly compressible. Acceptable compression and disintegration attributes were achieved and 125 MPa was nominated for bulk tablet manufacture, with an acceptable range of 100-150 MPa (FIG. 16). Compression pressure of 125 MPa was nominated for bulk tablet manufacture.

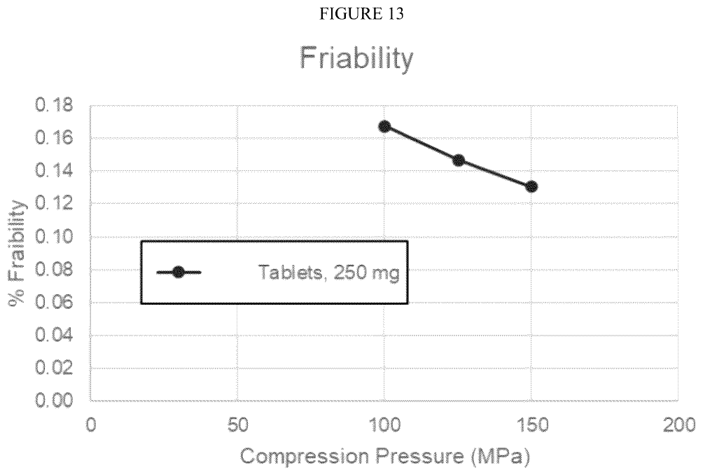

The tablet friability was very low over the tested pressure range. FIG. 13 provides friability plot for 250 mg demonstration batch tablets where very low friability was observed over 100-150 MPa compression pressure range.

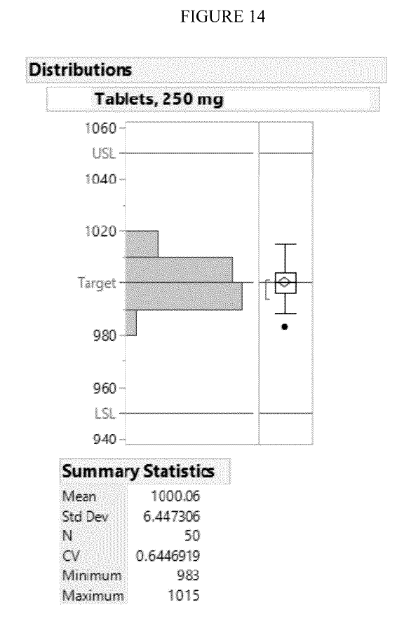

A weight analysis of bulk tablets was performed by weight a composite sample of 50 tablets. Weight control of Compound 1A Final blend was excellent, average tablet weight was 1000 mg and % RSD was 0.64. No tablets outside 2% of target weight were observed. FIG. 14 shows weight distribution where upper specification limit (USL) and lower specification limit (LSL) are ±5% of target.

The Tablet tooling was inspected for material accumulation after the completion of the run. Approximately 400 tablets were compressed on each station and very slight buildup was observed. Material that did accumulate was easily removed with a dry wipe.

The tablet appearance also remained consistent through the run and was white to off-white in color with glossy finish. Zero tablet defects were observed. Overall, the new formulation and process were highly successful in the manufacture of 250 mg tablets. A batch summary can be seen in Table 30.

TABLE 30
Batch summary of Tablets, 250 mg demonstration batch.

| | Compound 1A |
|---|---|
| Tablet Batch | Tablets, 250 mg |
| # of Stations | 2 |
| Tooling Shape | 0.3750″ × 0.7480″ |
| | Modified Oval |
| Target Tablet Weight (mg) | 1000 ± 50 |
| Fill Cam (mm) | 0-19 |
| Batch Size (g) | 1000 |
| Batch Run Time (min) | ~15 |
| Turret Speed (rpm) | 20 |
| Feed Frame (rpm) | 12 |
| Pre-Compression (N) | 100-200 |
| Compression Pressure (MPa) | 125 |
| Compression Force (kN) | 15.0 |
| Ejection Force (N) | 275 |
| Tablet Breaking Force (kP) | 26.5 |
| Tablet Tensile Strength (MPa) | 2.00 |
| Tablet Thickness (mm) | 7.09 |
| Ave. Tablet Weight (mg) | 1000.0 |
| Standard Deviation (mg) | 6.45 |
| % RSD | 0.64 |
| Disintegration Time (min:sec) | 7:07 |
| Friability (% Loss) | 0.1468 |
| ~Number of Tablets Available | 610 |

### Analytical Characterization of Demonstration Batch Tablets

The demonstration batch tablets were characterized by appearance, sink dissolution, water content by KF, identification by retention time and UV spectra, content uniformity (CU), and assay by HPLC. Results are summarized in the Certificate of Testing in Appendix A.

Immediate release Compound 1A tablets at 250 mg strength were successfully developed and scaled for CTM manufacture. Compound 1A was found to be difficult to process due to its poor flow and tendency to stick to tablet tooling during compression. Several formulation and process iterations were implemented before manufacture was successful. Using the compaction simulator in combination with the roller compacter saved a significant amount of material and resulted a high throughput process. Final blend was free flowing and processed through the rotary tablet press with no issues. Bulk tablets had a tensile strength value of 2.00 MPa, disintegration of ˜7 minutes, and very low friability. Analytical characterization of tablets showed on target potency and full dissolution at the 10 minute timepoint.

A summary of various trials for the tablet formulations is provided in Table F and Table G below:

TABLE F

| | | | | | | | | | | | Avicel | | |
|---|---|---|---|---|---|---|---|---|---|---|---|---|---|
| | | | | | | | | | | | (MCC) | | |
| Tablet or | | | | PH | PH | PH | Pearlitol | | Parteck | | PH | | |
| granula- | | Batch | % | 105 | 102 | 200 | 25C | | M200 | | 200 | | |
| tion | Batch | size | Comp. | (25 | (100 | (200 | (25 | Parteck | (200 | PRUV | (200 | Ac-Di-Sol | PRUV |
| lot # | description | (g) | 1A | μm) | μm) | μm) | μm) | M100 | μm) | (SSF) | μm) | (Croscarmellose) | (SSF) |
| 1 | Prototype | 25 | 49.25 | — | 22.66 | — | — | 22.66 | — | 0.5 | — | — | 1.5 |
| 2 | Prototype | 25 | 19.7 | — | 37.43 | — | — | 37.43 | — | 0.5 | — | — | 1.5 |
| 3 | Demo 1 | 950 | 50 | 17.25 | — | — | — | 17.25 | — | 1 | 7 | 3 | 1 |
| 4 | Confirmation | 400 | 50 | 17.25 | — | — | — | 17.25 | — | 1 | 7 | 3 | 1 |
| 5 | Reformulation | 75 | 50 | 17.25 | — | — | 17.25 | — | — | 1 | 7 | 3 | 1 |
| | trial | | | | | | | | | | | | |
| 6 | Reformulation | 75 | 50 | — | — | 17.25 | — | — | 17.25 | 1 | 7 | 3 | 1 |
| | trial | | | | | | | | | | | | |
| 7 | Demo 2 | 500 | 50 | — | — | 17.25 | — | — | 17.25 | 1 | 7 | 3 | 1 |
| 8 | Punch stick | 37 | 27 | — | 28.25 | — | — | 28.25 | — | 1 | 7 | 3 | 2 |
| | evaluation | | | | | | | | | | | | |
| 9 | Demo 3 | 1000 | 27 | — | 28.25 | — | — | 28.25 | — | 1 | 7 | 3 | 2 |
| 10 | Final batch | 10899 | 27 | — | 28.3 | — | — | 28.25 | — | 1 | 7 | 3 | 2 |

TABLE G

| | | | | Intra | Intra | Intra | Intra |
|---|---|---|---|---|---|---|---|
| Tablet or | | % | | granular | granular | granular | granular |
| granulation | | Comp. | | density | blend tapped | Blend True | blend |
| lot # | Aliases | 1 A | Intra granular blending steps | Blend bulk | density | Density | Hausner ratio |
| 1 | Prototype batch (250 mg) | 50 | blend, 25 mesh screen, blend | 0.18 | | 1.55 | |
| 2 | Prototype batch (100 mg) | 20 | blend, 25 mesh screen, blend | 0.27 | | 1.53 | |
| 3 | Demo batch 1 | 50 | blend, comil, blend | 0.38 | 0.64 | 1.45 | 1.68 |
| 4 | Confirmation batch w/GMP | 50 | blend, comil, blend | 0.11 | 0.23 | | 2.09 |
| | API | | | | | | |
| 5 | Reform trials (large/small | 50 | blend, 25 mesh screen, blend | 0.17 | 0.31 | | 1.82 |
| | PSD) | | | | | | |
| 6 | Reform trials (large/small | 50 | blend, 25 mesh screen, blend | 0.18 | 0.3 | | 1.67 |
| | PSD) | | | | | | |
| 7 | Demo batch 2, no de-lumping | 50 | blend in bin blender, no | 0.26 | 0.38 | 1.53 | 1.46 |
| | | | delump | | | | |
| 8 | Demo batch 3, scale up | 27 | blend in bin blender, no | 0.34 | 0.52 | 1.52 | 1.53 |
| | | | delump | | | | |

Table H below provides flow characteristics for the tablet formulations:

TABLE H

| Carr's | | |
|---|---|---|
| compressibility | Hausner | Description |
| index (%) | ratio | of flow |
| <10 | 1.00-1.11 | Excellent |
| 11-15 | 1.12-1.18 | Good |
| 16-20 | 1.19-1.25 | Fair |
| 21-25 | 1.26-1.34 | Passable |

### Example 3: High Drug Load Formulations

Tablet prototype formulations were evaluated with the goal to increase drug concentration from the formulation of Example 2. Formulations from 50-80% Compound 1A were evaluated and the formulation containing 70% Compound 1A was nominated for scale up. Overall, tablets at three doses strengths (250, 500, and 750 mg) were manufactured.

### Tablet Reformulation and Demonstration Batch

### Reformulation Trials

The goal of the reformulation efforts was to increase the concentration of Compound 1A in the tablet, ultimately increasing the available dose for clinical trials. Table 31 shows the low dose formulation compared to the reformulation trials, where Compound 1 A concentration was increased from 50 to 80%. Formulations were evaluated based on flow properties, tablet physical characteristics, and determine the risk of material sticking and accumulating on tablet tooling. In this example, percent compositions are described on a weight:weight basis, unless otherwise specified.

TABLE 31
Low drug load formulation compared to reformulation trials where Compound 1 A percentage was increased to 50 to 80%

| | | Low drug load | | | | |
|---|---|---|---|---|---|---|
| | | Formulation | Reformulation 1 | Reformulation 2 | Reformulation 3 | Reformulation 4 |
| Component | Function | (wt. %) | (wt. %) | (wt. %) | (wt. %) | (wt. %) |
| Compound 1A | Drug Carrier | 27.00 | 50.00 | 60.00 | 70.00 | 80.00 |
| API | | | | | | |
| (92.8% Assay) | | | | | | |
| AVICEL ® PH | Filler/Diluent | 28.25 | 16.00 | 11.00 | 6.00 | 2.50 |
| 102 | | | | | | |
| (Microcrystalline | | | | | | |
| Cellulose) | | | | | | |
| PARTECK | Filler/Diluent | 28.25 | 16.00 | 11.00 | 6.00 | 2.50 |
| M100 | | | | | | |
| (Mannitol) | | | | | | |
| AC-DI-SOL ® | Disintegrant | 3.00 | 3.00 | 3.00 | 3.00 | 3.00 |
| (Croscarmellose | | | | | | |
| Sodium) | | | | | | |
| CAB-O-SIL ® | Glidant | 0.50 | 1.00 | 1.00 | 1.00 | 1.00 |
| (Fumed | | | | | | |
| Silica) | | | | | | |
| Sodium Stearyl | Lubricant | 1.00 | 1.00 | 1.00 | 1.00 | 1.00 |
| Fumarate | | | | | | |
| (PRUV ®) | | | | | | |
| Intra-Granular Total: | | 88.00 | 87.00 | 87.00 | 87.00 | 90.00 |
| AVICEL ® PH- | Compression | 7.00 | 8.00 | 8.00 | 8.00 | 5.00 |
| 200 | Aid | | | | | |
| (Microcrystalline | | | | | | |
| Cellulose) | | | | | | |
| AC-DI-SOL ® | Disintegrant | 3.00 | 3.00 | 3.00 | 3.00 | 3.00 |
| (Croscarmellose | | | | | | |
| Sodium) | | | | | | |
| Sodium Stearyl | Lubricant | 2.00 | 2.00 | 2.00 | 2.00 | 2.00 |
| Fumarate | | | | | | |
| (PRUV ®) | | | | | | |

| | | | | | |
|---|---|---|---|---|---|
| Extra-Granular Total: | 12.00 | 13.00 | 13.00 | 13.00 | 10.00 |
| Total: | 100.00 | 100.00 | 100.00 | 100.00 | 100.00 |

Prototype reformulation batches were manufactured via slug and mill using a single station hand press. Slugs were compressed to a solid fraction of 0.65 using 0.8750″ flat faced round tooling. Slug weights were ˜400 mg and were milled using a hand mill with 1000 μm mesh screen. The resulting granulation then underwent final blending to incorporate extra-granular excipients. Final blend was then compressed into tablets using a single station hand press.

Final blends were characterized for bulk, tapped, and true densities. The resulting Hausner ratios were similar for all, indicating that 80% Compound 1 A is capable of forming a free-flowing final blend suitable for high-speed tablet manufacturing (Table 32).

TABLE 32
Final blend characterization data for
the four prototype reformulation blends

| | 50% | 60% | 70% | 80% |
|---|---|---|---|---|
| | Compound | Compound | Compound | Compound |
| Blend | 1A | 1A | 1A | 1A |
| True Density | 1.57 | 1.56 | 1.58 | 1.59 |
| Slug Solid | 0.65 | 0.65 | 0.66 | 0.67 |
| Fraction | | | | |
| Bulk Density | 0.47 | 0.47 | 0.49 | 0.48 |
| (g/cc) | | | | |
| Tapped Density | 0.67 | 0.67 | 0.67 | 0.67 |
| (g/cc) | | | | |
| Hausner Ratio | 1.43 | 1.43 | 1.37 | 1.40 |
| Carr Index | 29.58 | 29.85 | 26.87 | 28.36 |

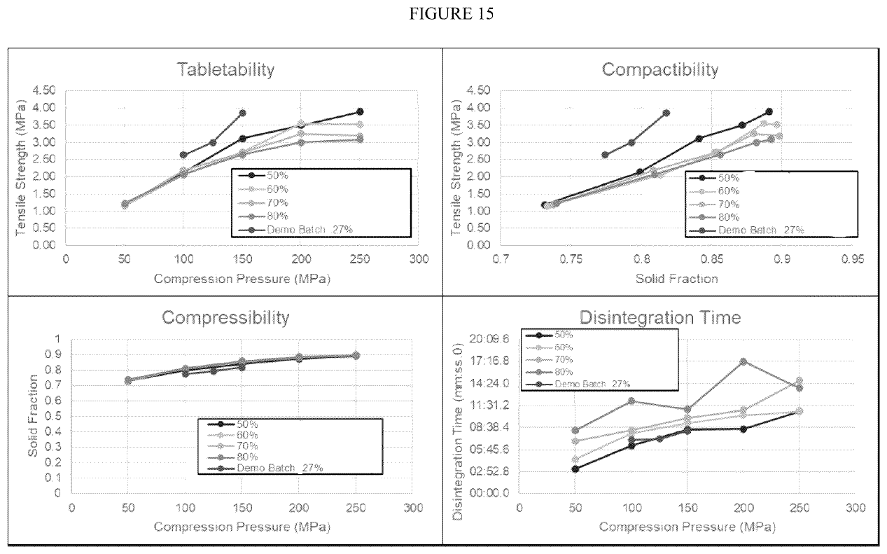

Final blends were compressed into tablets at 1000 mg total weight using 0.3750″×0.7480″ modified oval tooling. Tablet formulations maintain high level of compressibility with increasing drug loading and appear to perform similarly in all compression trials. No indication of over compression was observed during manufacture or break force testing. Disintegration times increased with increasing drug loading and ranged from 3-15 minutes, overall. All formulations were compressed at 75 MPa pressure for analytical characterization which resulted in a tensile strength of ˜1.75 MPa and disintegration times of ˜4-10 minutes. Compression and disintegration profiles can be seen in FIG. 15 along with the low dose formulation that contains 27% Compound 1A.

A punch sticking evaluation was completed by compressing 200 tablets of each formulation on the single station press, then evaluating the punch tips and tablets visually. Tablets were compressed using the STYL'One Evolution compaction simulator with 0.2500″ SRC tooling at 100 mg total tablet weight. The Korsch XL100 press profile was used at 30 rpm to match the unit for CTM manufacture. No powder accumulation was observed on the punch tips and tablets maintained a smooth glossy finish for all formulations. Tablets generated from the punch sticking study were also tested for friability where very low loss and no defects were observed for all formulations.

Table 33 provides friability data from prototype Compound 1A tablet formulations. Tablets were manufactured at 150 MPa compression pressure and have very low friability.

TABLE 33

| | 50% | 60% | 70% | 80% |
|---|---|---|---|---|
| | Compound | Compound | Compound | Compound |
| Blend | 1A | 1A | 1A | 1A |
| Friability (%) | 0.1675 | 0.1226 | 0.1520 | 0.1685 |

Heckel analysis was performed on the 80% Compound 1A final blend as a “worst case scenario” to determine if strain rate sensitivity was a potential issue during high-speed tablet manufacturing. Blend is compressed under slow and fast conditions, where the slow setting is 1 mm/s compression speed and fast is 300 mm/s. No capping, lamination, or other compression defects were observed at fast or slow compression rates and breaking forces remained consistent. The formulation had a low strain rate sensitivity of 11.87% and was characterized as a moderately hard/brittle material.

All tablet formulations were compressed using 0.3750″×0.7480″ modified oval tooling with total tablet weight of 1000 mg. Based on compression and disintegration data, all formulations were compressed at 75 MPa compression pressure. The effective tablet strengths after correcting for the 92.8% use potency value can be seen in Table 34 below.

TABLE 34
Compound 1A prototype formulation and strengths
that were passed along for analytical testing

| | Strength |
|---|---|
| Tablet Formulation | (mg Compound 1A) |
| 50% Compound 1A | 464 |
| 60% Compound 1A | 557 |
| 70% Compound 1A | 650 |
| 80% Compound 1A | 742 |

### Compound 1A Prototype Tablet Analytical Testing

The four tablet formulations were characterized by appearance, non-sink dissolution in biorelevant media, and assay and impurities by HPLC. All prototype reformulation tablets were off-white oval tablets in physical appearance.

TABLE 35
Appearance Data for Prototype Tablets

| Formulation | Appearance |
|---|---|
| 464 mgA | Off-White Oval Tablet |
| 557 mgA | Off-White Oval Tablet |
| 650 mgA | Off-White Oval Tablet |
| 742 mgA | Off-White Oval Tablet |

The dissolution performance of the Prototype tablets was tested in a non-sink dissolution in biorelevant media experiment against unformulated Compound 1A. All tablets performed similar to each other and the unformulated API. Tablet disintegration occurred nearly instantaneously. Lower drug load formulations saw greater gastric exposure than higher drug load tablets. No significant difference in performance was observed upon the addition of FaSSIF at the 30 minute mark, with all tablet formulations and the API reaching full dissolution at the 70 minute time point.

TABLE 36
Dissolution results

| | Cmax GB | Cmax IB | AUC35-210 IB | C210 |
|---|---|---|---|---|
| Formulation | (μgA/mL) | (μgA/mL) | (min*μgA/mL) | (μgA/mL) |
| 464 mgA | 1517 | 1097 | 187900 | 1097 |
| 557 mgA | 1518 | 1071 | 183900 | 1071 |
| 650 mgA | 1403 | 1103 | 181400 | 1061 |
| 742 mgA | 1227 | 1142 | 189600 | 1125 |
| Compound 1A API | 610 | 1175 | 183600 | 1175 |

Assay/Impurities analysis of the PK tablets by HPLC shows similar total relative substances compared with the bulk API (Table 37), indicating that no chemical degradation occurred during the tablet manufacturing processes.

TABLE 37
Assay/Impurities of Compound 1A Prototype Tablets Compared with Bulk API

| | | | Compound | Compound | Compound | Compound |
|---|---|---|---|---|---|---|
| | Compound 1A | Compound | 1A Tablets, | 1A Tablets, | 1A Tablets, | 1A Tablets, |
| RRT | (reference std) | 1A API | 464 mg Active | 557 mg Active | 650 mg Active | 742 mg Active |
| 0.90 | 0.07% | 0.07% | 0.06% | 0.06% | 0.06% | 0.06% |
| 1.10 | | 0.09% | 0.08% | 0.08% | 0.07% | 0.07% |
| 1.71 | | | <0.05% | <0.05% | <0.05% | <0.05% |
| 1.78 | | <0.05% | | | | |
| 1.91 | <0.05% | | | | | |
| 1.94 | <0.05% | | | | | |
| 1.96 | <0.05% | | | | | |
| 2.30 | 0.38% | | 0.35% | 0.36% | 0.36% | 0.36% |
| 2.40 | | 0.28% | | | | |
| 2.67 | 0.07% | | | | | |
| 2.77 | | | <0.05% | <0.05% | <0.05% | <0.05% |
| Total | 0.53% | 0.44% | 0.49% | 0.50% | 0.50% | 0.49% |
| Impurities | | | | | | |
| % Label | — | — | 98.5% ± 0.9% | 99.8% ± 2.4% | 100.5% ± 1.0% | 98.2% ± 1.3% |
| Claim | | | | | | |

Dynamic vapor sorption (DVS) indicated that the Compound/A tablet blend adsorbs a significant amount of water at elevated humidity levels. While there is some hysteresis in the DVS curve of the tablet blend, it does not appear to convert to a stable hydrate form given the subsequent rapid water desorption. FIG. 16 provides a plot of % weight versus relative humidity for the tablet blend.

### Granulation Scale-Up

The previously identified 70% Compound 1A granulation formulation was nominated for scale-up and granulation development on the pilot scale. A summary of the scale-up tablet formulation can be found in Table 38.

TABLE 38
Scale-up Tablet Formulation Summary

| | | Unit | | | |
|---|---|---|---|---|---|
| | | Composition | 750 mg | 500 mg | 250 mg |
| Component | Function | (wt. %) | (mg/tab) | (mg/tab) | (mg/tab) |
| Compound 1A API | Drug Carrier | 70.00 | 808.19 | 538.79 | 269.40 |
| (92.8% Assay) | | | | | |
| AVICEL ® PH-102 | Filler/Diluent | 6.00 | 69.27 | 46.18 | 23.09 |
| (Microcrystalline | | | | | |
| Cellulose) | | | | | |
| PARTECK M100 | Filler/Diluent | 6.00 | 69.27 | 46.18 | 23.09 |
| (Mannitol) | | | | | |
| AC-DI-SOL ® | Disintegrant | 3.00 | 34.64 | 23.09 | 11.55 |
| (Croscarmellose | | | | | |
| Sodium) | | | | | |
| CAB-O-SIL ® | Glidant | 1.00 | 11.55 | 7.70 | 3.85 |
| (Fumed Silica) | | | | | |
| Sodium Stearyl | Lubricant | 1.00 | 11.55 | 7.70 | 3.85 |
| Fumarate (PRUV ®) | | | | | |
| Intra-Granular Total: | | 87.00 | 1004.46 | 669.64 | 334.82 |
| AVICEL ® PH-200 | Compression Aid | 8.00 | 92.36 | 61.58 | 30.79 |
| (Microcrystalline | | | | | |
| Cellulose) | | | | | |
| AC-DI-SOL ® | Disintegrant | 3.00 | 34.64 | 23.09 | 11.55 |
| (Croscarmellose | | | | | |
| Sodium) | | | | | |
| Sodium Stearyl | Lubricant | 2.00 | 23.09 | 15.39 | 7.70 |
| Fumarate (PRUV ®) | | | | | |

| | | | | |
|---|---|---|---|---|
| Extra-Granular Total: | 13.00 | 150.09 | 100.06 | 50.03 |
| Total: | 100.00 | 1154.56 | 769.70 | 384.85 |

| | | | |
|---|---|---|---|
| Target Dose (mg Compound 1A): | 750 | 500 | 250 |

The scale-up blend was evaluated at multiple processing conditions for bulk powder properties and compressibility. The primary processing parameter investigated was press force, ranging from 4.0 to 6.0 kN/cm. A summary of the pilot scale granulation conditions can be found in Table 39.

TABLE 39
Scale-up Roller Compaction Processing Parameter Summary

| Parameter | Trial 1 | Trial 2 | Trial 3 | Trial 4 |
|---|---|---|---|---|
| Roll Type | Knurled | Knurled | Knurled | Knurled |
| Press Force (kN/cm) | 5.0 | 4.0 | 5.0 | 6.0 |
| Gap (mm) | 2.5 | 2.5 | 2.5 | 2.5 |
| Roll Speed (rpm) | 2.5 | 1.5 | 1.5 | 1.5 |
| Granulator Screen | 1.0 mm | 1.0 mm | 1.0 mm | 1.0 mm |
| | wire | wire | wire | wire |
| Granulator Type | Star Rotor | Star Rotor | Star Rotor | Star Rotor |
| Granulator Speed, | 50 | 50 | 50 | 50 |
| CW (rpm) | | | | |
| Granulator Speed, | 50 | 50 | 50 | 50 |
| CCW (rpm) | | | | |

All scale-up processing conditions at 1.5 rpm resulted in hard/brittle ribbons that easily passed through the granulator screen. Conditions at 2.5 rpm roll speed resulted in an auger feed that could not keep up due to the low bulk density of the intragranular blend. The granulation process resulted in significant increases in density and improved powder flow.

All scale-up conditions resulted in granules with very similar bulk and tapped density along with flow properties. Scale-up Trial 3 conditions were nominated based on having the lowest ribbon solid fraction which will ensure more compressibility during tablet manufacturing.

### Demonstration Batch Common Granulation

The demonstration batch final blend and tablet composition is the same as the composition provided in Table 38. The 70% Compound 1A demonstration batch common granulation was manufactured using “Trial 3” from the scale-up development (press force of 4.0 kN/cm). A summary of the demonstration batch granulation processing conditions can be found in Table 40, the granulation bulk flow properties can be found in Table 41.

TABLE 40
Demonstration Batch Common Granulation,
700 mg/g, Processing Parameters

| Parameter | Lead Process Condition |
|---|---|
| Roll Type | Knurled |
| Press Force (kN/cm) | 4.0 kN/cm |
| Gap (mm) | 2.5 |
| Roll Speed (rpm) | 1.5 |
| Granulator Screen | 1.0 mm wire |
| Granulator Type | Star Rotor |
| Granulator Speed, CW (rpm) | 50 |
| Granulator Speed, CCW (rpm) | 50 |

TABLE 41
Common Granulation, 700 mg/g, Bulk Material Properties

| | Bulk | Final |
|---|---|---|
| Blend Sample | Granulation | Blend |
| Bulk Density (g/cc) | 0.47 | 0.54 |
| Tapped Density (g/cc) | 0.66 | 0.70 |
| Hausner Ratio | 1.40 | 1.30 |
| True Density (g/cc) | 1.58 | 1.51 |
| Shear Cell Flow Function | NA | 10.4 |

The addition of extra-granular excipients has increased fines and improved flow based on the Hausner Ratio.

### Demonstration Batch Tablet Compression

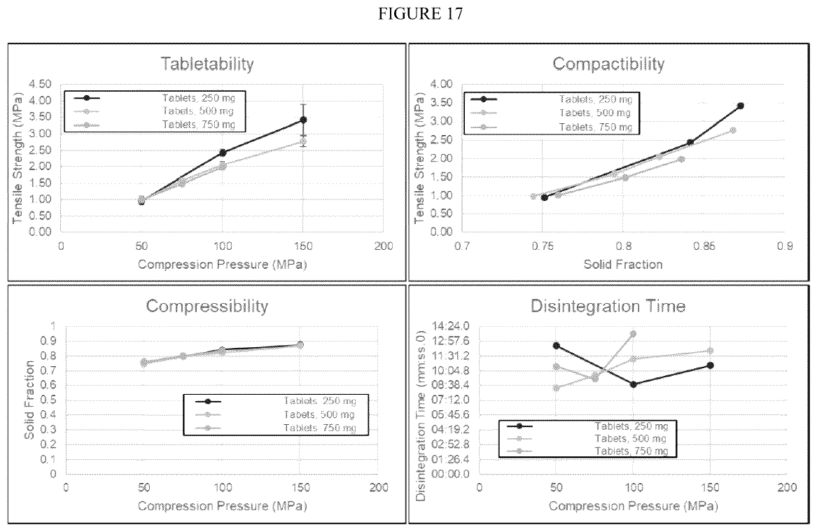

Compound A common granulation, 700 mg/g, was used to manufacture tablets at three strengths (250, 500, and 750 mg). Demonstration batch tablets, 250 mg, were evaluated at a total batch size of approximately 1000 tablets using 0.3750″ SRC tablet tooling. Physical tablet characteristics were tested at compression forces of 50, 100, and 150 MPa, resulting in tablets with acceptable hardness and disintegration, with 100 MPa being the best performing (FIG. 17). See Table 42 for a summary of physical tablet characterization. Demonstration batch tablets, 500 mg, were evaluated at a total batch size of approximately 1000 tablets using 0.3252″×0.7265″ modified oval tooling. Physical tablet characteristics were tested at compression forces of 50, 75, 100, and 150 MPa, resulting in tablets with acceptable hardness and disintegration when compressed at 75 MPa (FIG. 17). See Table 42 for a summary of physical tablet characterization.

Demonstration batch tablets, 750 mg, were evaluated at a total batch size of approximately 1000 tablets using 0.3750×0.7480″ modified oval tooling. Physical tablet characteristics were tested at compression forces of 50, 75, and 100 MPa, resulting in tablets with acceptable hardness and disintegration when compressed at 75 MPa (FIG. 17). See Table 42 for a summary of physical tablet characterization.

TABLE 42
Demonstration Batch Tablet Physical Characterization

| | | | | | | | | Disintegration |
|---|---|---|---|---|---|---|---|---|
| | Dose | Compression | Compression | | | Avg. | | Time |
| | Strength | Pressure | Force | Weight | Thickness | Hardness | Friability | [First, Last] |
| ID | (mg) | (MPa) | (kN) | (mg) | (mm) | (kP) | (%) | (mm:ss) |
| Demonstration | 250 | 50 | 3.6 | 386 | 5.68 | 7.7 | 0.259 | 07:20, 08:00^{a} |
| Tablets | | 100 | 7.1 | 400 | 5.31 | 18.0 | 0.194 | 07:45, 09:45 |
| | | 150 | 10.7 | 382 | 4.94 | 23.1 | 0.076 | 10:00, 11:10 |
| Demonstration | 500 | 50 | 6.8 | 768 | 6.37 | 11.2 | 0.168 | 08:09, 08:37 |
| Tablets | | 75 | 10.2 | 781 | 6.13 | 17.5 | 0.192 | 08:42, 10:30 |
| | | 100 | 13.6 | 765 | 5.87 | 20.7 | 0.117 | 09:55, 12:30 |
| | | 150 | 20.4 | 772 | 5.67 | 26.5 | 0.116 | 11:50, 12:13 |
| Demonstration | 750 | 50 | 7.5 | 1151 | 8.35 | 16.9 | 0.174^{b} | 09:25, 11:32 |
| Tablets | | 75 | 11.2 | 1154 | 8.02 | 23.6 | 0.078 | 11:25, 14:30 |
| | | 100 | 15.0 | 1173 | 7.86 | 30.5 | 0.068 | 13:01, 14:22 |

^{a}5 out of 6 tablets disintegrated within 8 minutes. One tablet took 17:40.
^{b}major defects observed in multiple tablets.

A summary of the demonstration batch processing parameters for tablets, 250, 500, and 750 mg, is shown in Table 43. These operating conditions are anticipated to result in a robust tablet formulation when transferred to GMP manufacturing on the Korsch XL-100 tablet press.

TABLE 43
Demonstration Batch Critical Processing Parameters and Physical Tablet Properties

| | Compound 1A | Compound 1A | Compound 1A |
|---|---|---|---|
| Product Name | Tablets, 250 mg | Tablets, 500 mg | Tablets, 500 mg |
| Tooling | 0.3750″ SRC | 0.3525″ × 0.7265″ | 0.3750″ × 0.7480″ |
| | | Mod Oval | Mod Oval |
| Target Tablet Weight (mg) | 385 ± 19 mg | 770 ± 38 mg | 1155 ± 57 mg |
| Number of Stations | 2 | 2 | 2 |
| Fill Cam (mm) | 0-12 | 0-19 | 0-19 |
| Feed Frame Set-Point | 2.0 | 2.0 | 2.0 |
| Turret Speed (rpm) | 20 | 20 | 20 |
| Pre-compression force (N) | 20 | 20 | 30 |
| Main compression force (kN) | 7.1 | 10.2 | 11.2 |
| Main Compression Pressure (MPa) | 100 | 75 | 75 |
| Ejection Force (N) | 160 | 190 | 240 |
| % Yield | 75 | 82 | 86 |
| % Accountability | 88 | 95 | 97 |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed:

1. A pharmaceutical composition comprising:
a. 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate having the following formula: 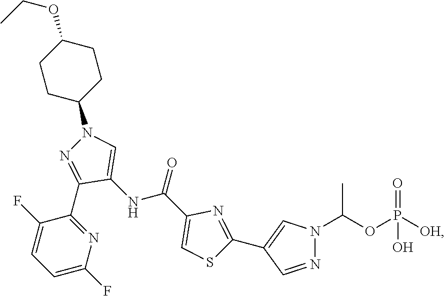 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]
or a stereoisomer or mixture of stereoisomers, a pharmaceutically acceptable salt, tautomer, solvate, hydrate, co-crystal, clathrate, or polymorph thereof (Compound 1), wherein Compound 1 is present in an amount from about 15% to about 35% by weight based on total weight of the pharmaceutical composition;
b. intra-granular excipients from about 50% to about 70% by weight based on total weight of the pharmaceutical composition; and
c. extra-granular excipients from about 10% to about 20% by weight based on total weight of the pharmaceutical composition.

2. The pharmaceutical composition of claim 1, wherein Compound 1 is 4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt having the following formula:

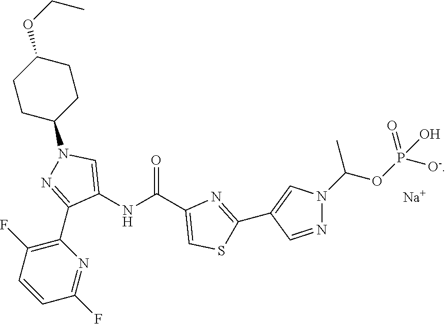

3. The pharmaceutical composition of claim 1, wherein Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate having the following formula:

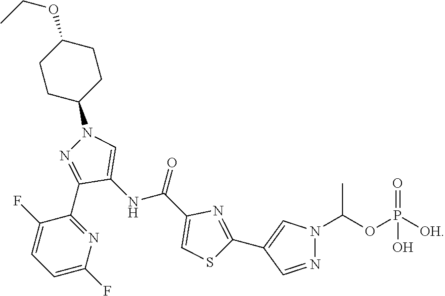 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

4. The pharmaceutical composition of claim 1, wherein the intra-granular excipients comprise one or more of a diluent, a disintegrant, a glidant and a lubricant.

5. The pharmaceutical composition of claim 4, wherein the diluent comprises one or more of microcrystalline cellulose and mannitol.

6. The pharmaceutical composition of claim 1, wherein the extra-granular excipient comprises from a compression aid, a disintegrant, a glidant and a lubricant.

7. A tablet comprising the pharmaceutical composition of claim 1.

8. The tablet of claim 7, wherein the tablet comprises about 154 mg or about 269.39 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

9. The tablet of claim 7, wherein the tablet comprises:
about 269.39 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 283.20 mg microcrystalline cellulose, about 282.50 mg mannitol, about 30 mg croscarmellose sodium, about 5 mg fumed silica, and about 10 mg sodium stearyl fumarate; and
the extra-granular excipient comprising 70 mg microcrystalline cellulose, about 30 mg croscarmellose sodium, and about 20 mg sodium stearyl fumarate.

10. The tablet of claim 7, wherein the tablet comprises:
about 250 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 292.50 mg microcrystalline cellulose, about 292.50 mg mannitol, about 30 mg croscarmellose sodium, about 5 mg fumed silica, and about 10 mg sodium stearyl fumarate; and
the extra-granular excipient comprising 70 mg microcrystalline cellulose, about 30 mg croscarmellose sodium, and about 20 mg sodium stearyl fumarate.

11. The tablet of claim 10, wherein the tablet comprises:
about 154 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 173.47 mg microcrystalline cellulose, about 173.47 mg mannitol, about 18 mg croscarmellose sodium, about 3 mg fumed silica, and about 6 mg sodium stearyl fumarate; and
the extra-granular excipient comprising 42 mg microcrystalline cellulose, about 18 mg croscarmellose sodium, and about 12 mg sodium stearyl fumarate.

12. The tablet of claim 7, wherein the tablet comprises:
about 150 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
the intra-granular excipients comprising about 175.50 mg microcrystalline cellulose, about 175.50 mg mannitol, about 18 mg croscarmellose sodium, about 3 mg fumed silica, and about 6 mg sodium stearyl fumarate; and
the extra-granular excipient comprising 42 mg microcrystalline cellulose, about 18 mg croscarmellose sodium, and about 12 mg sodium stearyl fumarate.

13. A method for preparing the tablet of claim 7, wherein the method comprises blending Compound 1 with an intra-granular excipient and an extra-granular excipient.

14. A method of treating a disease comprising administering to a subject in need thereof the pharmaceutical composition of claim 1, wherein the disease is an auto-immune disease, an inflammatory disorder, a cardiovascular disease, a nerve disorder, a neurodegenerative disorder, an allergic disorder, asthma, pancreatitis, multi-organ failure, a kidney disease, platelet aggregation, cancer, transplantation, sperm motility, erythrocyte deficiency, graft rejection, a lung injury, a respiratory disease, an ischemic condition, bacterial infection or a viral infection.

15. The method of claim 14, wherein disease is a cytokine release syndrome, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, IL-1 driven disorders, a MyD88 driven disorder, primary cutaneous T-cell lymphoma, chronic lymphocytic leukemia, smoldering or indolent multiple myeloma, leukemia, acute myeloid leukemia, DLBCL, ABC DLBCL, chronic lymphocytic lymphoma, primary effusion lymphoma, Burkitt lymphoma/leukemia, acute lymphocytic leukemia, B-cell prolymphocytic leukemia, lymphoplasmacytic lymphoma, myelodysplastic syndrome, clonal cytopenia of undetermined significance, chronic myelomonocytic leukemia, myelofibrosis, polycythemia vera, Kaposi's sarcoma, Waldenstrom's macroglobulinemia, splenic marginal zone lymphoma, multiple myeloma, plasmacytoma or intravascular large B-cell lymphoma.

16. The method of claim 14, wherein disease is a myelodysplastic syndrome, optionally the myelodysplastic syndrome is a higher-risk myelodysplastic or a lower-risk myelodysplastic syndrome.

17. The method of claim 15, wherein the myelodysplastic syndrome is refractory or relapsed myelodysplastic syndrome.

18. The method of claim 14, wherein the method further comprises administering a second therapeutic agent.

19. A pharmaceutical composition comprising:
a. 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate having the following formula: 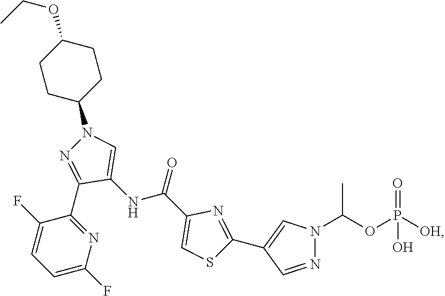 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]
or a stereoisomer or mixture of stereoisomers, a pharmaceutically acceptable salt, tautomer, solvate, hydrate, co-crystal, clathrate, or polymorph thereof (Compound 1), wherein Compound 1 is present in an amount from about 65% to about 85% by weight based on total weight of the pharmaceutical composition;
b. intra-granular excipients from about 15% to about 20% by weight based on total weight of the pharmaceutical composition; and
c. extra-granular excipients from about 10% to about 15% by weight based on total weight of the pharmaceutical composition.

20. A method of treating a disease comprising administering to a subject in need thereof the pharmaceutical composition of claim 19, wherein the disease is an auto-immune disease, an inflammatory disorder, a cardiovascular disease, a nerve disorder, a neurodegenerative disorder, an allergic disorder, asthma, pancreatitis, multi-organ failure, a kidney disease, platelet aggregation, cancer, transplantation, sperm motility, erythrocyte deficiency, graft rejection, a lung injury, a respiratory disease, an ischemic condition, bacterial infection or a viral infection.
